(19) United States
(12) Patent Application Publication
(10) Pub. No.: US 2012/0156230 A1
(43) Pub. Date: Jun. 21, 2012

(54) TREATMENT OF SPINAL CORD INJURY AND TRAUMATIC BRAIN INJURY USING PLACENTAL STEM CELLS

(76) Inventors: Stewart Abbot, Warren, NJ (US); James W. Edinger, Belford, NJ (US); Aleksandar Francki, Annandale, NJ (US); Vladimir Jankovic, New York, NY (US); Aleksandr Kaplunovsky, Budd Lake, NJ (US); Kristen Labazzo, Springfield, NJ (US); Eric Law, East Brunswick, NJ (US); Bitao Liang, Closter, NJ (US)

(21) Appl. No.: 13/327,245

(22) Filed: Dec. 15, 2011

Related U.S. Application Data
(60) Provisional application No. 61/424,559, filed on Dec. 17, 2010.

(51) Int. Cl. A61K 39/00; A61P 37/06; A61P 37/02; A61K 35/50; A61P 25/00

(52) U.S. Cl. 424/184.1; 424/93.7; 424/583

(57) ABSTRACT

Provided herein are methods of treatment of individuals having an injury to the central nervous system, such as a spinal cord injury or a traumatic brain injury, using placental stem cells and placental multipotent stem cells described herein, and populations of such placental cells.

This application claims priority to U.S. provisional application No. 61/424,559, filed Dec. 17, 2010, the disclosure of which is herein incorporated by reference in its entirety.

### 1. FIELD

Provided herein are methods of using human placental stem cells to treat individuals having a traumatic spinal cord injury (SCI) or a traumatic brain injury (TBI).

### 2. BACKGROUND

Central Nervous System (CNS) injuries represent a medically important problem. Approximately 300,000 people living in the United States suffer from spinal cord injury (SCI), and each year, approximately 10.000-14,000 new cases of SCI are diagnosed. SCI usually results from trauma to the vertebral column, e.g., as a result of displaced bone or disc compressing the spinal cord. SCI can occur without obvious vertebral fractures, for example, from loss of blood flow to the spinal cord, and spinal fractures can occur without SCI.

Traumatic brain injury (TBI) is one of the leading causes of disability and death among young adults around the world. In military situations, for example, brain damage results from, e.g., direct impact, penetrating objects such as bullets and shrapnel, and from blast waves caused by explosions.

### 3. SUMMARY

Provided herein are methods of treating, managing, and/or ameliorating disorders and/or conditions associated with CNS injury. In one embodiment, provided herein is a method of treating an individual having a traumatic CNS injury, or a disease, disorder or condition associated with CNS injury, comprising administering to the individual a therapeutically effective amount of placental stem cells, or medium conditioned by placental stem cells, wherein the therapeutically effective amount is an amount sufficient to cause a detectable improvement in one or more symptoms of, or a reduction in the progression of one or more symptoms of, said traumatic CNS injury, or a disease, disorder or condition associated with said CNS injury. Also provided herein is the use of placental stem cells in the manufacture of a medicament for treating, managing, and/or ameliorating one or more symptoms of a CNS injury, e.g., SCI or TBI.

In some embodiments, the therapeutically effective amount of placental stem cells, or culture medium conditioned by placental stem cells is administered to the individual within 1, 2, 3, 4, 5, 6, 7, 8, 9, 10, 11, 12, 13, 13, 15, 16, 17, 18, 19, 20, 25, 30, 35, 40, 45, 50 days or more of injury, or within 1, 2, 3, 4, 5, 6, 7, 8, 9, 10 or more years after the CNS injury. In some embodiments, the therapeutically effective amount of placental stem cells, or culture medium conditioned by placental stem cells is administered to the individual within 21 days, 14 days, or 7 days of the CNS injury, or within 48 hours, 24 hours, 12 hours or 3 hours of the CNS injury.

In a specific embodiment, the CNS injury is an SCI. In some embodiments, the SCI is caused by direct trauma. In some embodiments, the SCI is caused by compression by bone fragments, hematoma, or disc material. In some embodiments, the SCI is at one or more of the cervical vertebrae, thoracic vertebrae, lumbar vertebrae, or sacral vertebrae. In some embodiments, the SCI is to one or more of the cervical cord, thoracic cord, lumbrosacral vertebrae, conus, occiput, or one or more nerves of the cauda equina.

In some embodiments, the disease, disorder or condition associated with CNS injury is spinal shock resulting from an SCI. In some embodiments, the disease, disorder or condition associated with CNS injury is neurogenic shock resulting from an SCI. In some embodiments, the disease, disorder or condition associated with CNS injury is autonomic dysreflexia resulting from an SCI. In some embodiments, the disease, disorder or condition associated with CNS injury is edema resulting from an SCI. In some embodiments, the disease, disorder or condition associated with CNS injury is selected from the group consisting of central cord syndrome, Brown-Séquard syndrome, anterior cord syndrome, conus medullaris syndrome, and cauda equina syndrome.

In some embodiments, the therapeutically effective amount of placental stem cells, or medium conditioned by placental stem cells administered is an amount sufficient to cause a detectable improvement in, or a reduction in the progression of, one or more of the following symptoms of SCI: loss or impairment of motor function, sensory function, or motor and sensory function, in the cervical, thoracic, lumbar or sacral segments of the spinal cord. In some embodiments, the one or symptoms of the SCI comprises loss or impairment of motor function, sensory function, or motor and sensory function, in the arms, trunk, legs or pelvic organs. In some embodiments, the one or symptoms of the SCI comprises numbness in one or more of dermatomes C1, C2, C3, C4, C5, C6, C7, T1, T2, T3, T4, T5, T6, T7, T8, T9, T10, T11, T12, L1, L2, L3, L4 or L5.

In some embodiments of treating SCI provided herein, the method further comprises administering a second therapeutic agent to said individual. In some embodiments, the second therapeutic agent is a corticosteroid, a neuroprotective agent, an immunomodulatory or immunosuppressant agent, or an anticoagulant.

In another specific embodiment of the methods of treatment provided herein, the disease, disorder or condition associated with CNS injury is a TBI. In some embodiments, the TBI is an injury to the frontal lobe, parietal lobe, occipital lobe, temporal lobe, brain stem, or cerebellum. In some embodiments, the TBI is a mild TBI. In some embodiments, the TBI is a moderate to severe TBI.

In some embodiments, the therapeutically effective amount of placental stem cells, or medium conditioned by placental stem cells administered is an amount sufficient to cause a detectable improvement in, or a reduction in the progression of, one or more of the following symptoms of mild TBI: headache, memory problems, attention deficits, mood swings and frustration, fatigue, visual disturbances, memory loss, poor attention/concentration, sleep disturbances, dizziness/loss of balance, irritability, emotional disturbances, feelings of depression, seizures, nausea, loss of smell, sensitivity to light and sounds, mood changes, getting lost or confused, or slowness in thinking.

In some embodiments, the therapeutically effective amount of placental stem cells, or medium conditioned by placental stem cells administered is an amount sufficient to cause a detectable improvement in, or a reduction in the progression of, one or more of the following symptoms of moderate to severe TBI: difficulties with attention, difficulties with concentration, distractibility, difficulties with memory, slowness of speed of processing, confusion, perseveration, impulsiveness, difficulties with language processing, difficulties with speech and language, not understanding the spoken word (receptive aphasia), difficulty speaking and being understood (expressive aphasia), slurred speech, speaking very fast or very slow, problems reading, problems writing, difficulties with interpretation of touch, temperature, movement, limb position and fine discrimination, difficulty with the integration or patterning of sensory impressions into psychologically meaningful data, partial or total loss of vision, weakness of eye muscles and double vision (diplopia), blurred vision, problems judging distance, involuntary eye movements (nystagmus), intolerance of light (photophobia), a decrease or loss of hearing, ringing in the ears (tinnitus), increased sensitivity to sounds, loss or diminished sense of smell (anosmia), loss or diminished sense of taste, seizures, convulsions associated with epilepsy, physical paralysis/spasticity, chronic pain, loss of control of bowel and/or bladder, sleep disorders, loss of stamina, appetite changes, dysregulation of body temperature, menstrual difficulties, social-emotional difficulties, dependent behaviors, lack of emotional ability, lack of motivation, irritability, aggression, depression, disinhibition, or lack of awareness.

In some embodiments of treating TBI provided herein, the method further comprises administering a second therapeutic agent to said individual. In some embodiments, the second therapeutic agent is an anti-seizure drug, an antidepressant, amantadine, methylphenidate, bromocriptine, carbamamazapine or amitriptyline.

In some embodiments of treating a CNS injury, e.g., an SCI or TBI, as provided herein, the therapeutically effective amount of placental stem cells, or culture medium conditioned by placental stem cells is administered to the individual by a route selected from the group consisting of intravenous, intraarterial, intraperitoneal, intraventricular, intrasternal, intracranial, intramuscular, intrasynovial, intraocular, intravitreal, intracerebral, intracerebroventricular, intrathecal, intraosseous infusion, intravesical, transdermal, intracisternal, epidural, lumbar puncture, cisterna magna or subcutaneous administration. In some embodiments, the therapeutically effective amount of placental stem cells, or culture medium conditioned by placental stem cells is administered to the individual directly into the site of the injury.

In a specific embodiment, said placental stem cells are CD10⁺, CD34⁻, CD105⁺, CD200⁺ placental stem cells. In another specific embodiment, said placental stem cells express CD200 and do not express HLA-G; or express CD73, CD105, and CD200; or express CD200 and OCT-4; or express CD73 and CD105 and do not express HLA-G; or express CD73 and CD105 and facilitate the formation of one or more embryoid-like bodies in a population of placental cells comprising said stem cell when said population is cultured under conditions that allow for the formation of an embryoid-like body; or express OCT-4 and facilitate the formation of one or more embryoid-like bodies in a population of placental cells comprising said stem cell when said population is cultured under conditions that allow for the formation of an embryoid-like body. In certain embodiments, the placental stem cells suppress the activity of an immune cell, e.g., suppress proliferation of a T cell.

In certain embodiments, provided herein is a method of inhibiting a pro-inflammatory response to a CNS injury in an individual, for example an SCI or TBI, comprising contacting T cells (e.g., CD4⁺ T lymphocytes or leukocytes) that are associated with or part of the CNS injury with placental stem cells, e.g., the placental stem cells described herein. In a specific embodiment, the inflammatory response is a Th1 response or a Th17 response. In a specific embodiment, said contacting detectably reduces Th1 cell maturation. In a specific embodiment of the method, said contacting detectably reduces the production of one or more of interleukin-1β(IL-1β), IL-12, IL-17, IL-21, IL-23, tumor necrosis factor alpha (TNFα) and/or interferon gamma (IFNγ) by said T cells. In another specific embodiment of the method, said contacting potentiates or upregulates a regulatory T cell (Treg) phenotype. In another specific embodiment, said contacting downregulates dendritic cell (DC) and/or macrophage expression of markers (e.g., CD80, CD83, CD86, ICAM-1, HLA-II) that promote Th1 and/or Th17 immune response. In a specific embodiment, said T cells are also contacted with IL-10, e.g., exogenous IL-10 or IL-10 not produced by said T cells, e.g., recombinant IL-10. In another embodiment, provided herein is a method of reducing the production of pro-inflammatory cytokines from macrophages, comprising contacting the macrophages with an effective amount of placental stem cells. In another embodiment, provided herein is a method of upregulating tolerogenic cells and/or cytokines, e.g., from macrophages, comprising contacting immune system cells with an effective amount of placental stem cells. In a specific embodiment, said contacting causes activated macrophages to produce detectably more IL-10 than activated macrophages not contacted with said placental stem cells. In another embodiment, provided herein is a method of upregulating, or increasing the number of, anti-inflammatory T cells, comprising contacting immune system cells with an effective amount of placental stem cells.

In one embodiment, provided herein is a method of inhibiting a CNS injury-associated Th1 response in an individual comprising administering to the individual an effective amount of placental stem cells, wherein said effective amount is an amount that results in a detectable decrease in said CNS injury-associated Th1 response in the individual. In another embodiment, provided herein is a method of inhibiting a CNS injury-associated Th17 response in an individual comprising administering to the individual an effective amount of placental stem cells, wherein said effective amount is an amount that results in a detectable decrease in a Th17 response in the individual. In specific embodiments of these methods, said administering detectably reduces the production, by T cells, or an antigen presenting cell (e.g., DC, macrophage or monocyte) in said individual, of one or more of lymphotoxins-1α (LT-1α), IL-1β, IL-12, IL-17, IL-21, IL-23, TNFα and/or IFNγ. In another specific embodiment of the method, said contacting potentiates or upregulates a regulatory T cell (Treg). In another embodiment, said contacting modulates (e.g., reduces) production by dendritic cells (DC) and/or macrophages in said individual of markers that promote a Th1 or Th17 response (e.g., CD80, CD83, CD86, ICAM-1, HLA-II). In another specific embodiment, the method comprises additionally administering IL-10 to said individual.

In another aspect, provided herein are placental stem cells, as described herein, that have been genetically engineered to express one or more anti-inflammatory cytokines. In a specific embodiment, said anti-inflammatory cytokines comprise IL-10.

### 3.1 Definitions

As used herein, the term “about,” when referring to a stated numeric value, indicates a value within plus or minus 10% of the stated numeric value.

As used herein, the term “amount,” when referring to the placental stem cells described herein, means a particular number of placental cells, for example, a number of placental stem cells that is administered in one or more doses that is sufficient, e.g., to cause a detectable improvement in, reduce the severity of, or reduce the progression of, one or more symptoms of a CNS injury.

As used herein, the term “derived” means isolated from or otherwise purified. For example, placental derived adherent cells are isolated from placenta. The term “derived” encompasses cells that are cultured from cells isolated directly from a tissue, e.g., the placenta, and cells cultured or expanded from primary isolates.

As used herein, “immunolocalization” means the detection of a compound, e.g., a cellular marker, using an immune protein, e.g., an antibody or fragment thereof in, for example, flow cytometry, fluorescence-activated cell sorting, magnetic cell sorting, in situ hybridization, immunohistochemistry, or the like.

As used herein, the term “SH2” refers to an antibody that binds an epitope on the marker CD105. Thus, cells that are referred to as SH2⁺ are CD105⁺.

As used herein, the terms “SH3” and SH4” refer to antibodies that bind epitopes present on the marker CD73. Thus, cells that are referred to as SH3⁻ and/or SH4⁺ are CD73⁺.

As used herein, a stem cell is “isolated” if at least 50%, 60%, 70%, 80%, 90%, 95%, or at least 99% of the other cells with which the stem cell is naturally associated are removed from the stem cell, e.g., during collection and/or culture of the stem cell. A population of “isolated” cells means a population of cells that is substantially separated from other cells of the tissue, e.g., placenta, from which the population of cells is derived. In some embodiments, a population of, e.g., stem cells is “isolated” if at least 50%, 60%, 70%, 80%, 90%, 95%, or at least 99% of the cells with which the population of stem cells are naturally associated are removed from the population of stem cells, e.g., during collection and/or culture of the population of stem cells.

As used herein, the term “placental stem cell” refers to a stem cell or progenitor cell that is derived from, e.g., isolated from, a mammalian placenta, regardless of morphology, cell surface markers, or the number of passages after a primary culture, which adheres to a tissue culture substrate (e.g., tissue culture plastic or a fibronectin-coated tissue culture plate). The term “placenta stem cell” as used herein does not, however, refer to a trophoblast, a cytotrophoblast, embryonic germ cell, or embryonic stem cell, as those cells are understood by persons of skill in the art. A cell is considered a “stem cell” if the cell retains at least one attribute of a stem cell, e.g., a marker or gene expression profile associated with one or more types of stem cells; the ability to replicate at least 10-40 times in culture; multipotency, e.g., the ability to differentiate, either in vitro, in vivo or both, into cells of one or more of the three germ layers; the lack of adult (i.e., differentiated) cell characteristics, or the like. The terms “placental stem cell” and “placenta-derived stem cell” may be used interchangeably. Unless otherwise noted herein, the term “placental” includes the umbilical cord. The placental stem cells disclosed herein are, in certain embodiments, multipotent in vitro (that is, the cells differentiate in vitro under differentiating conditions), multipotent in vivo (that is, the cells differentiate in vivo), or both.

As used herein, a stem cell is “positive” for a particular marker when that marker is detectable. For example, a placental stem cell is positive for, e.g., CD73 because CD73 is detectable on placental stem cells in an amount detectably greater than background (in comparison to, e.g., an isotype control or an experimental negative control for any given assay). A cell is also positive for a marker when that marker can be used to distinguish the cell from at least one other cell type, or can be used to select or isolate the cell when present or expressed by the cell.

As used herein, “immunomodulation” and “immunomodulatory” mean causing, or having the capacity to cause, a detectable change in an immune response, and the ability to cause a detectable change in an immune response.

As used herein, “immunosuppression” and “immunosuppressive” mean causing, or having the capacity to cause, a detectable reduction in an immune response, and the ability to cause a detectable suppression of an immune response.

### 4. BRIEF DESCRIPTION OF THE FIGURES

FIG. 1 shows the secretion of selected angiogenic proteins by placental derived adherent cells.

FIG. 2 shows the angiogenic effect of placental derived adherent cell-conditioned medium on Human Endothelial Cell (HUVEC) tube formation.

FIG. 3 shows the angiogenic effect of placental derived adherent cell-conditioned medium on Human Endothelial Cell migration.

FIG. 4 shows the effect of placental derived adherent cell-conditioned medium on Human Endothelial Cell proliferation.

FIG. 5 shows tube formation of HUVECs and placental derived adherent cells.

FIG. 6 shows the secretion of VEGF and IL-8 by placental derived adherent cells under hypoxic and normoxic conditions.

FIG. 7 shows positive effect of PDACs on angiogenesis in a chick chorioallantois angiogenesis model. bFGF: basic fibroblast growth factor (positive control). MDAMB231: Angiogenic breast cancer cell line (positive control). Y axis: Degree of blood vessel formation.

FIG. 8 shows positive effect of PDAC-conditioned medium (supernatants) on angiogenesis in a chick chorioallantois angiogenesis model. bFGF: basic fibroblast growth factor (positive control). MDAMB231: Angiogenic breast cancer cell line (positive control). Y axis: Degree of blood vessel formation.

FIG. 9: Hydrogen peroxide-generated reactive oxygen species present in cultures of astrocytes, or co-cultures of astrocytes and PDACs. RFU ROS activity: Relative fluorescence units for reactive oxygen species.

### 5. DETAILED DESCRIPTION

### 5.1 Methods of Treating a CNS Injury

Provided herein are methods for the treatment of an individual having an injury to the CNS, e.g., an SCI or TBI, or a disease, disorder or condition associated with CNS injury, comprising administering to the individual having the CNS injury one or more doses of placental stem cells. Methods for the treatment of such individuals, and for the administration of such stem cells, alone or in combination with other therapies, are discussed in detail below.

### 5.1.1 Treatment of Spinal Cord Injury (SCI)

Provided herein are methods of treating an individual having, or experiencing, a symptom of, or a disease disorder or condition related to, an SCI, comprising administering to the individual a therapeutically effective amount of placental stem cells, or medium conditioned by placental stem cells, wherein the therapeutically effective amount is an amount sufficient to cause a detectable improvement in one or more symptoms of, or a reduction in the progression of one or more symptoms of, said SCI. As used herein, “one or more symptoms” includes objectively measurable parameters, such as degree of inflammation, immune response, gene expression within the site of injury that is correlated with the healing process, quality and extent of scarring at the site of injury, improvement in the patient's motor and sensory function, etc., and subjectively measurable parameters, such as patient well-being, patient perception of improvement in motor and sensory function, perception of lessening of pain or discomfort associated with the SCI, and the like.

SCI is an insult to the spinal cord resulting in a change, either temporary or permanent, in its normal motor, sensory, or autonomic function. SCI includes conditions known as tetraplegia (formerly known as quadriplegia) and paraplegia. Thus, in some embodiments of the method of treatment of SCI provided herein, the individual having, or experiencing, a symptom of, or a disease disorder or condition related to, an SCI is tetraplegic or paraplegic.

Tetraplegia refers to injury to the spinal cord in the cervical region, characterized by impairment or loss of motor and/or sensory function in the cervical segments of the spinal cord due to damage of neural elements within the spinal canal. Tetraplegia results in impairment of function in the arms as well as in the trunk, legs and pelvic organs. It does not include brachial plexus lesions or injury to peripheral nerves outside the neural canal.

Paraplegia refers to impairment or loss of motor and/or sensory function in the thoracic, lumbar or sacral (but not cervical) segments of the spinal cord, secondary to damage of neural elements within the spinal canal. With paraplegia, arm functioning is spared, but, depending on the level of injury, the trunk, legs and pelvic organs may be involved. The term is used in referring to cauda equina and conus medullaris injuries, but not to lumbosacral plexus lesions or injury to peripheral nerves outside the neural canal.

Common causes of SCI include, but are not limited to, motor vehicle accidents, falls, violence, sports injuries, vascular disorders, tumors, infectious conditions, spondylosis, latrogenic injuries (especially after spinal injections and epidural catheter placement), vertebral fractures secondary to osteoporosis, and developmental disorders.

In certain embodiments, the SCI can result from, e.g., blunt force trauma, compression, displacement, or the like. In certain embodiments, the spinal cord is completely severed. In certain other embodiments, the spinal cord is damaged, e.g., partially severed, but not completely severed. In other embodiments, the spinal cord is compressed, e.g., through damage to the bony structure of the spinal column, displacement of one or more vertebrae relative to other vertebrae, inflammation or swelling of adjacent tissues, or the like.

In one embodiment, the SCI is at one or more of the cervical vertebrae. In another embodiment, the SCI is at one or more of the thoracic vertebrae. In another embodiment, the SCI is at one or more of the lumbar vertebrae. In another embodiment, the SCI is at one or more of the sacral vertebrae. In certain embodiments, the SCI is at vertebra C1, C2, C3, C4, C5, C6 or C7; or at vertebra T1, T2, T3, T4, T5, T6, T7, T8, T9, T10, T11 or T12; or at vertebra L1, L2, L3, L4 or L5. In certain other embodiments, the SCI is to a spinal root exiting the spinal column between C1 and C2; between C2 and C3; Between C3 and C4; between C4 and C5; between C5 and C6; between C6 and C7; between C7 and T1; between T1 and T2; between T2 and T3; between T3 and T4; between T4 and T5; between T5 and T6; between T6 and T7; between T7 and T8; between T8 and T9; between T9 and T10; between T10 and T11; between T11 and T12; between T12 and L1; between L1 and L2; between L2 and L3; between L3 and L4; or between L4 and L5. In certain embodiments, the injury is to the cervical cord. In other embodiments, the injury is to the thoracic cord. In other embodiments the SCI is to the lumbrosacral cord. In certain other embodiments, the SCI is to the conus. In certain other embodiments, the CNS injury is to one or more nerves in the cauda equina. In another embodiment, the SCI is at the occiput.

In certain embodiments, a symptom of an SCI is numbness in one or more dermatomes (i.e., a patch of skin innervated by a given spinal cord level). In specific embodiments, the symptom of an SCI is numbness in one or more of dermatomes C1, C2, C3, C4, C5, C6, C7, T1, T2, T3, T4, T5, T6, T7, T8, T9, T10, T11, T12, L1, L2, L3, L4 or L5.

Spinal shock is a state of transient physiologic (rather than anatomic) reflex depression of cord function below the level of injury, with associated loss of all sensorimotor functions. An initial increase in blood pressure due to the release of catecholamines, followed by hypotension, is noted. Flaccid paralysis, including of the bowel and/or bladder, is observed, and sometimes sustained priapism develops. These symptoms tend to last several hours to days until the reflex arcs below the level of the injury begin to function again (e.g., bulbocavernosus reflex, muscle stretch reflex [MSR]). Therefore, in specific embodiments of the method, the therapeutically effective amount of placental stem cells is an amount sufficient to cause a detectable improvement in one or more symptoms of spinal shock resulting from SCI, including, but not limited to, loss of some or all sensorimotor function, high blood pressure, hypotension, flaccid paralysis (e.g., of the bowel and/or bladder), and priapism.

Neurogenic shock is manifested by the triad of hypotension, bradycardia, and hypothermia. Shock tends to occur more commonly in injuries above T6, secondary to the disruption of the sympathetic outflow from T1-L2 and to unopposed vagal tone, leading to a decrease in vascular resistance, with associated vascular dilatation. Neurogenic shock is distinct from spinal and hypovolemic shock, which tends to be associated with tachycardia. Thus, in some embodiments of the method of treating SCI, the therapeutically effective amount of placental stem cells is an amount sufficient to cause a detectable improvement in one or more symptoms of neurogenic shock resulting from SCI, including, but not limited to, hypotension, bradycardia, hypothermia, a decrease in vascular resistance, and vascular dilatation.

Autonomic dysreflexia (AD) is a syndrome of massive imbalanced reflex sympathetic discharge occurring in patients with SCI above the splanchnic sympathetic outflow (T5-T6). AD occurs after the phase of spinal shock in which reflexes return. Individuals with injury above the major splanchnic outflow may develop AD. Below the injury, intact peripheral sensory nerves transmit impulses that ascend in the spinothalamic and posterior columns to stimulate sympathetic neurons located in the intermediolateral gray matter of the spinal cord. The inhibitory outflow above the SCI from cerebral vasomotor centers is increased, but it is unable to pass below the block of the SCI. This large sympathetic outflow causes release of various neurotransmitters (norepinephrine, dopamine-b-hydroxylase, dopamine), causing piloerection, skin pallor, and severe vasoconstriction in arterial vasculature. The result is sudden elevation in blood pressure and vasodilation above the level of injury. Patients commonly have a headache caused by vasodilation of pain sensitive intracranial vessels. Thus, in some embodiments of the method of treating SCI, the therapeutically effective amount of placental stem cells is an amount sufficient to cause a detectable improvement in one or more symptoms of autonomic dysreflexia resulting from SCI, including, but not limited to, piloerection, skin pallor, severe vasoconstriction in arterial vasculature, elevation in blood pressure, and vasodilation above the level of injury.

In some embodiments of the method of treating SCI, the therapeutically effective amount of placental stem cells is an amount sufficient to cause a detectable improvement in one or more symptoms of edema resulting from SCI. In some embodiments of the method, the therapeutically effective amount of placental stem cells is an amount sufficient to cause a detectable improvement in one or more symptoms of SCI caused by direct trauma. In some embodiments of the method, the therapeutically effective amount of placental stem cells is an amount sufficient to cause a detectable improvement in one or more symptoms of SCI caused by compression by vertebral bone fragments. In some embodiments of the method, the therapeutically effective amount of placental stem cells is an amount sufficient to cause a detectable improvement in one or more symptoms of SCI caused by compression of vertebral disc material.

The methods of treating SCI provided herein also provide for the treatment of an individual having, or experiencing, a symptom of, or a disease disorder or condition related to, other classifications of SCI including, but not limited to, central cord syndrome, Brown-Séquard syndrome, anterior cord syndrome, conus medullaris syndrome, and cauda equina syndrome.

Central cord syndrome often is associated with a cervical region injury and leads to greater weakness in the upper limbs than in the lower limbs, with sacral sensory sparing. Thus, in specific embodiments of the method of treating SCI, the therapeutically effective amount of placental stem cells is an amount sufficient to cause a detectable improvement in one or more symptoms of central cord syndrome, including, but not limited to, greater weakness in the upper limbs than in the lower limbs, with sacral sensory sparing.

Brown-Séquard syndrome, which often is associated with a hemisection lesion of the cord, causes a relatively greater ipsilateral proprioceptive and motor loss, with contralateral loss of sensitivity to pain and temperature. Thus, in specific embodiments of the method of treating SCI, the therapeutically effective amount of placental stem cells is an amount sufficient to cause a detectable improvement in one or more symptoms of Brown-Séquard syndrome, including, but not limited to, ipsilateral proprioceptive and motor loss, with contralateral loss of sensitivity to pain and temperature.

Anterior cord syndrome often is associated with a lesion causing variable loss of motor function and sensitivity to pain and temperature; proprioception is preserved. Thus, in specific embodiments of the method of treating SCI, the therapeutically effective amount of placental stem cells is an amount sufficient to cause a detectable improvement in one or more symptoms of anterior cord syndrome, including, but not limited to, variable loss of motor function and sensitivity to pain and temperature.

Conus medullaris syndrome is associated with injury to the sacral cord and lumbar nerve roots leading to areflexic bladder, bowel, and lower limbs, while the sacral segments occasionally may show preserved reflexes (e.g., bulbocavernosus and micturition reflexes). Thus, in specific embodiments of the method of treating SCI, the therapeutically effective amount of placental stem cells is an amount sufficient to cause a detectable improvement in one or more symptoms of conus medullaris syndrome, including, but not limited to, areflexic bladder, bowel, and lower limbs.

Cauda equina syndrome is due to injury to the lumbosacral nerve roots in the spinal canal, leading to areflexic bladder, bowel, and lower limbs. Thus, in specific embodiments of the method of treating SCI, the therapeutically effective amount of placental stem cells is an amount sufficient to cause a detectable improvement in one or more symptoms of cauda equina syndrome, including, but not limited to, areflexic bladder, bowel, and lower limbs.

In certain embodiments, the particular technique(s) for detecting an improvement in, a reduction in the severity of, or a reduction in the progression of, one or more symptoms, conditions, or syndromes of SCI is not critical to the method of treating SCI provided herein. In certain embodiments, the assessment of said improvement or reduction in the progression of one or more symptoms, conditions, or syndromes of SCI is determined according to the judgment of the practitioner in the art. In certain embodiments, the assessment of said improvement or reduction in the progression of one or more symptoms, conditions, or syndromes of SCI is determined according to the judgment of the practitioner in the art in combination with the subjective experience of the subject.

In some embodiments, an improvement in one or more symptoms of, or a reduction in the progression of one or more symptoms of, said SCI is detected in accordance with the International Standards for Neurological and Functional Classification of Spinal Cord Injury. The International Standards for Neurological and Functional Classification of Spinal Cord Injury, published by the American Spinal Injury Association (ASIA), is a widely accepted system describing the level and extent of SCI based on a systematic motor and sensory examination of neurologic function. See International Standards For Neurological Classification Of Spinal Cord Injury, J Spinal Cord Med. 26 Suppl 1:S50-6 (2003), the disclosure of which is hereby incorporated by reference in its entirety.

In particular embodiments, an improvement in one or more symptoms of, or a reduction in the progression of one or more symptoms of, said SCI is detected in accordance with the ASIA Impairment Scale (modified from the Frankel classification), using the following categories:
A—Complete: No sensory or motor function is preserved in sacral segments S4-S5.4.
“Complete” refers to the absence of sensory and motor functions in the lowest sacral segments.
B—Incomplete: Sensory, but not motor, function is preserved below the neurologic level and extends through sacral segments S4-S5. “Incomplete” refers to preservation of sensory or motor function below the level of injury, including the lowest sacral segments.
C—Incomplete: Motor function is preserved below the neurologic level, and most key muscles below the neurologic level have muscle grade less than 3.
D—Incomplete: Motor function is preserved below the neurologic level, and most key muscles below the neurologic level have muscle grade greater than or equal to 3.
E—Normal: Sensory and motor functions are normal.

Thus, in a specific embodiment of the method of treating SCI provided herein, the therapeutically effective amount of placental stem cells (e.g., PDACs) is an amount sufficient to cause a decrease in impairment according to the ASIA impairment scale (AIS). In some embodiments, the decrease is a one, two, three, four or five grade reduction in impairment, wherein one grade corresponds to a single category improvement, for example, a reduction in impairment from category D to category E. In some embodiments, the therapeutically effective amount of placental stem cells (e.g., PDACs) is an amount sufficient to convert an individual classified as ASIA A to ASIA B, ASIA C, ASIA D or ASIA E according to the AIS. In some embodiments, the therapeutically effective amount of placental stem cells (e.g., PDACs) is an amount sufficient to convert an individual classified as ASIA B to ASIA C, ASIA D or ASIA E according to the AIS. In some embodiments, the therapeutically effective amount of placental stem cells (e.g., PDACs) is an amount sufficient to convert an individual classified as ASIA C to ASIA D or ASIA E according to the AIS. In some embodiments, the therapeutically effective amount of placental stem cells (e.g., PDACs) is an amount sufficient to convert an individual classified as ASIA D to ASIA E according to the AIS.

In some embodiments, an improvement in one or more symptoms of, or a reduction in the progression of one or more symptoms of said SCI is detected by measuring the muscle strength of the patient. In some embodiments, muscle strength can be graded using the following Medical Research Council (MRC) scale of 0-5:

5—Normal power

4+—Submaximal movement against resistance

4—Moderate movement against resistance

4⁻—Slight movement against resistance

3—Movement against gravity but not against resistance

2—Movement with gravity eliminated

1—Flicker of movement

0—No movement

The following key muscles are tested in patients with SCI, and the corresponding level of injury is indicated:

C5—Elbow flexors (biceps, brachialis)

C6—Wrist extensors (extensor carpi radialis longus and brevis)

C7—Elbow extensors (triceps)

C8—Finger flexors (flexor digitorum profundus) to the middle finger

T1—Small finger abductors (abductor digiti minimi)

L2—Hip flexors (iliopsoas)

L3—Knee extensors (quadriceps)

L4—Ankle dorsiflexors (tibialis anterior)

L5—Long toe extensors (extensors hallucis longus)

S1—Ankle plantar flexors (gastrocnemius, soleus)

Thus, in a specific embodiment of the method of treating SCI provided herein, the therapeutically effective amount of placental stem cells (e.g., PDACs) is an amount sufficient to cause a one, two, three, four or five point increase in muscle strength according to the MRC scale. For example, in some embodiments, the therapeutically effective amount of placental stem cells (e.g., PDACs) is an amount sufficient to cause a muscle having no movement as a result of the SCI to have a flicker of movement, movement with gravity eliminated, movement against gravity but not against resistance, slight movement against resistance, moderate movement against resistance, submaximal movement against resistance, or normal power. In some embodiments, the therapeutically effective amount of placental stem cells (e.g., PDACs) is an amount sufficient to cause a muscle having only a flicker of movement as a result of the SCI to have movement with gravity eliminated, movement against gravity but not against resistance, slight movement against resistance, moderate movement against resistance, submaximal movement against resistance, or normal power. In some embodiments, the therapeutically effective amount of placental stem cells (e.g., PDACs) is an amount sufficient to cause a muscle having only movement with gravity eliminated as a result of the SCI to have movement against gravity but not against resistance, slight movement against resistance, moderate movement against resistance, submaximal movement against resistance, or normal power. In some embodiments, the therapeutically effective amount of placental stem cells (e.g., PDACs) is an amount sufficient to cause a muscle having only movement against gravity but not against resistance as a result of the SCI to have slight movement against resistance, moderate movement against resistance, submaximal movement against resistance, or normal power. In some embodiments, the therapeutically effective amount of placental stem cells (e.g., PDACs) is an amount sufficient to cause a muscle having only slight movement against resistance as a result of the SCI to have moderate movement against resistance, submaximal movement against resistance, or normal power. In some embodiments, the therapeutically effective amount of placental stem cells (e.g., PDACs) is an amount sufficient to cause a muscle having only moderate movement against resistance as a result of the SCI to have submaximal movement against resistance or normal power. In some embodiments, the therapeutically effective amount of placental stem cells (e.g., PDACs) is an amount sufficient to cause a muscle having only submaximal movement against resistance as a result of the SCI to have normal power.

In some embodiments, the therapeutically effective amount of placental stem cells (e.g., PDACs) is an amount sufficient to cause a one, two, three, four or five point increase in the strength of a biceps muscle of the subject. In some embodiments, the therapeutically effective amount of placental stem cells (e.g., PDACs) is an amount sufficient to cause a one, two, three, four or five point increase in the strength of a brachialis muscle of the subject. In some embodiments, the therapeutically effective amount of placental stem cells (e.g., PDACs) is an amount sufficient to cause a one, two, three, four or five point increase in the strength of a extensor carpi radialis longus or brevis muscle of the subject. In some embodiments, the therapeutically effective amount of placental stem cells (e.g., PDACs) is an amount sufficient to cause a one, two, three, four or five point increase in the strength of a triceps muscle of the subject. In some embodiments, the therapeutically effective amount of placental stem cells (e.g., PDACs) is an amount sufficient to cause a one, two, three, four or five point increase in the strength of a flexor digitorum profundus muscle of the subject. In some embodiments, the therapeutically effective amount of placental stem cells (e.g., PDACs) is an amount sufficient to cause a one, two, three, four or five point increase in the strength of a abductor digiti minimi muscle of the subject. In some embodiments, the therapeutically effective amount of placental stem cells (e.g., PDACs) is an amount sufficient to cause a one, two, three, four or five point increase in the strength of a iliopsoas muscle of the subject. In some embodiments, the therapeutically effective amount of placental stem cells (e.g., PDACs) is an amount sufficient to cause a one, two, three, four or five point increase in the strength of a quadriceps muscle of the subject. In some embodiments, the therapeutically effective amount of placental stem cells (e.g., PDACs) is an amount sufficient to cause a one, two, three, four or five point increase in the strength of a tibialis anterior muscle of the subject. In some embodiments, the therapeutically effective amount of placental stem cells (e.g., PDACs) is an amount sufficient to cause a one, two, three, four or five point increase in the strength of a extensors hallucis longus muscle of the subject. In some embodiments, the therapeutically effective amount of placental stem cells (e.g., PDACs) is an amount sufficient to cause a one, two, three, four or five point increase in the strength of a gastrocnemius or soleus muscle of the subject.

In some embodiments, an improvement in one or more symptoms of, or a reduction in the progression of one or more symptoms of, said SCI is detected by sensory testing. Sensory testing can be performed at the following levels:

C2—Occipital protuberance

C3—Supraclavicular fossa

C4—Top of the acromioclavicular joint

C5—Lateral side of antecubital fossa

C6—Thumb

C7—Middle finger

C8—Little finger

T1—Medial side of antecubital fossa

T2—Apex of axilla

T3—Third intercostal space (IS)

T4—Fourth IS at nipple line

T5—Fifth IS (midway between T4 and T6)

T6—Sixth IS at the level of the xiphisternum

T7—Seventh IS (midway between T6 and T8)

T8—Eighth IS (midway between T6 and T10)

T9—Ninth IS (midway between T8 and T10)

T10—10th IS or umbilicus

T11—11th IS (midway between T10 and T12)

T12—Midpoint of inguinal ligament

L1—Half the distance between T12 and L2

L2—Midanterior thigh

L3—Medial femoral condyle

L4—Medial malleolus

L5—Dorsum of the foot at third metatarsophalangeal joint

S1—Lateral heel

S2—Popliteal fossa in the midline

S3—Ischial tuberosity

S4-5—Perianal area (taken as 1 level)

Sensory scoring is for light touch and pinprick, as follows:

0—Absent

1—Impaired or hyperesthesia

2—Intact

A score of zero is given if the patient cannot differentiate between the point of a sharp pin and the dull edge. Thus, in a specific embodiment of the method of treatment provided herein, the therapeutically effective amount of placental stem cells (e.g., PDACs) is an amount sufficient to cause a one or two point increase in sensory scoring corresponding to one or more of C2, C3, C4, C5, C6, C7, C8, T1, T2, T3, T4, T5, T6, T7, T8, T9, T10, T11, T12, L1, L2, L3, L4, L5, S1, S2, S3, S4 and S5.

In some embodiments, an improvement in one or more symptoms of, or a reduction in the progression of one or more symptoms of, said SCI is detected by monitoring the daily life functionality of the patient. In some embodiments of the method of treatment of SCI provided herein, the therapeutically effective amount of placental stem cells (e.g., PDACs) is an amount sufficient to effect a functional improvement in the daily-life activities of the patient. In some embodiments, the Functional Independence Measure (FIM) is used to assess functional improvement of the patient. The FIM focuses on six areas of functioning: self-care, sphincter control, mobility, locomotion, communication and social cognition. Within each area, two or more specific activities/items are evaluated, with a total of 18 items. For example, six activity items (eating, grooming, bathing, dressing-upper body, dressing-lower body, and toileting) comprise the self-care area. Each of the 18 items is evaluated in terms of independence of functioning, using a seven-point scale:

Independent (No Human Assistance is Required):

7=Complete independence: The activity is typically performed safely, without modification, assistive devices or aids, and within reasonable time.

6=Modified independence: The activity requires an assistive device and/or more than reasonable time and/or is not performed safely.

Dependent (Human Supervision or Physical Assistance is Required):

5=Supervision or setup: No physical assistance is needed, but cuing, coaxing or setup is required.

4=Minimal contact assistance: Subject requires no more than touching and expends 75% or more of the effort required in the activity.

3=Moderate assistance: Subject requires more than touching and expends 50±75% of the effort required in the activity.

2=Maximal assistance: Subject expends 25±50% of the effort required in the activity.

1=Total assistance: Subject expends 0±25% of the effort required in the activity.

Thus, the FIM total score (summed across all items) estimates the cost of disability in terms of safety issues and of dependence on others and on technological devices. The profile of area scores and item scores pinpoints the specific aspects of daily living that have been most affected by SCI. In some embodiments of the method of treating SCI provided herein, the therapeutically effective amount of placental stem cells (e.g., PDACs) is an amount sufficient to cause a one, two, three, four, five or six point increase in functioning of the patient according to the FIM scale. In some embodiments, the therapeutically effective amount of placental stem cells (e.g., PDACs) is an amount sufficient to cause a subject requiring total assistance as a result of the SCI to require only moderate assistance, only minimal contact assistance, only supervision or setup, or to have modified independence or complete independence. In some embodiments, the therapeutically effective amount of placental stem cells (e.g., PDACs) is an amount sufficient to cause a subject requiring moderate assistance as a result of the SCI to require only minimal contact assistance, only supervision or setup, or to have modified independence or complete independence. In some embodiments, the therapeutically effective amount of placental stem cells (e.g., PDACs) is an amount sufficient to cause a subject requiring minimal contact assistance as a result of the SCI to require only supervision or setup, or to have modified independence or complete independence. In some embodiments, the therapeutically effective amount of placental stem cells (e.g., PDACs) is an amount sufficient to cause a subject requiring supervision or setup as a result of the SCI to have modified independence or complete independence. In some embodiments, the therapeutically effective amount of placental stem cells (e.g., PDACs) is an amount sufficient to cause a subject having modified independence as a result of the SCI to have complete independence.

An individual having, or experiencing, a symptom of, SCI, can be treated with a plurality of placental stem cells, and, optionally, one or more therapeutic agents, at any time during the progression of the injury. For example, the individual can be treated immediately after injury, or within 1, 2, 3, 4, 5, 6 days of injury, or within 1, 2, 3, 4, 5, 6, 7, 8, 9, 10, 11, 12, 13, 13, 15, 16, 17, 18, 19, 20, 25, 30, 35, 40, 45, 50 days or more of injury, or within 1, 2, 3, 4, 5, 6, 7, 8, 9, 10 or more years after injury. The individual can be treated once, or multiple times during the clinical course of the injury. In a specific embodiment of the method of treatment, said placental stem cells are administered to said individual within 21 days of development of one or more symptoms of an SCI. In another specific embodiment of the method of treatment, said placental stem cells are administered to said individual within 14 days of development of one or more symptoms of an SCI. In another specific embodiment of the method of treatment, said placental stem cells are administered to said individual within 7 days of development of one or more symptoms of an SCI. In another specific embodiment of the method of treatment, said placental stem cells are administered to said individual within 48 hours of development of one or more symptoms of an SCI. In another specific embodiment, said placental stem cells are administered to said individual within 24 hours of development of one or more symptoms of an SCI. In another specific embodiment, said placental stem cells are administered to said individual within 12 hours of development of one or more symptoms of an SCI. In another specific embodiment, said placental stem cells are administered to said individual within 3 hours of development of one or more symptoms of an SCI.

In certain embodiments of the invention, the individual is an animal, preferably a mammal, more preferably a non-human primate. In certain embodiments, the individual is a human patient. The individual can be a male or female subject. In certain embodiments, the subject is a non-human animal, such as, for instance, a cow, sheep, goat, horse, dog, cat, rabbit, rat or mouse.

The placenta stem cells useful in the treatment of SCI can be any of the placental stem cells disclosed herein (see Section 5.5). In a specific embodiment, the placental stem cells express CD200 and do not express HLA-G; express CD73, CD105, and CD200; express CD200 and OCT-4; express CD73 and CD105 and do not express HLA-G; express CD73 and CD105, and facilitate the formation of one or more embryoid-like bodies in a population of placental stem cells when said population is cultured under conditions that allow for the formation of embryoid-like bodies; or express OCT-4, and (c) facilitate the formation of one or more embryoid-like bodies in a population of placental stem cells when said population is cultured under conditions that allow for the formation of embryoid-like bodies; or any combination of the foregoing. In a specific embodiment, the placental stem cells are CD10⁺, CD105⁺, CD200⁺, CD34⁻ placental stem cells. In another specific embodiment, the placental stem cells are CD117⁻.

In one embodiment, the individual is administered a dose of about 300 million placental stem cells. Dosage, however, can vary according to the individual's physical characteristics, e.g., weight, and can range from 1 million to 10 billion placental stem cells per dose, preferably between 10 million and 1 billion per dose, or between 100 million and 500 million placental stem cells per dose. In some embodiments, the administration can be by any medically-acceptable route for the administration of live cells, e.g., intravenous, intraarterial, intraperitoneal, intraventricular, intrasternal, intracranial, intramuscular, intrasynovial, intraocular, intravitreal (e.g., where there is an ocular involvement), intracerebral, intracerebroventricular (e.g., where there is a neurologic or brain involvement), intrathecal, intraosseous infusion, intravesical, transdermal, intracisternal, epidural, or subcutaneous administration. In specific embodiments, administration is by bolus injection or infusion directly into the site of the SCI, e.g., via lumbar puncture.

In one embodiment, the placental stem cells are from a cell bank, e.g., a placental stem cell bank. In one embodiment, a dose of placental stem cells is contained within a blood bag or similar bag, suitable for bolus injection or administration by catheter.

Placental stem cells, or medium conditioned by placental stem cells, can be administered in a single dose, or in multiple doses. Where placental stem cells are administered in multiple doses, the doses can be part of a therapeutic regimen designed to relieve one or more acute symptoms of SCI, or can be part of a long-term therapeutic regimen designed to lessen the severity of SCI.

The methods for treating SCI provided herein further encompass treating SCI by administering a therapeutically effective amount of placental stem cells in conjunction with one or more therapies or treatments used in the course of treating SCI. The one or more additional therapies may be used prior to, concurrent with, or after administration of the placental stem cells. In some embodiments, the one or more additional therapies comprise the application of therapeutic spinal traction. Therapeutic spinal traction uses manually or mechanically created forces to stretch and mobilize the spine, based on the application of a force (usually a weight) along the longitudinal axis of the spinal column. If the neck or cervical segments are fractured, traction may straighten out and decompress the vertebral column.

In other embodiments, the one or more additional therapies comprise surgical stabilization of the spine, e.g. through the insertion of rods and screws to properly align the vertebral column or fuse adjacent vertebrae to strengthen the vertebra, promote bone re-growth, and reduce the likelihood of further SCI in the future. In other embodiments, the one or more additional therapies comprise rehabilitation (e.g., repetitive voluntary movement training, strength training, and the like), which can promote the formation of new local CNS connections. In other embodiments, the one or more additional therapies comprise functional electrical stimulation (FES) of specific nerves or muscles, for example, FES of phrenic nerves to assist breathing; FES of sacral roots to promote bladder and bowel function; FES of limb muscles to improve arm or hand function, as well as standing or walking.

Also provided herein are methods for the treatment of an individual having, or experiencing, a symptom of, SCI, comprising administering to the individual a plurality of placental stem cells sufficient to cause a detectable improvement in one or more symptoms, conditions, or syndromes of, or a reduction in the progression of one or more symptoms, conditions, or syndromes of, said SCI, and one or more therapeutic agents. In one embodiment, the therapeutic agent is corticosteroid. In other embodiments, the therapeutic agent is an anticoagulant, such as heparin. In other embodiments, the therapeutic agent is a neuroprotective agent. In some embodiments the neuroprotective agent is methylprednisolone sodium succinate (MPSS), GM-1 (Sygen), Gacylidine (GK-11), thyrotropin releasing hormone, monocycline (minocycline), lithium or erythropoietin (EPO).

In other embodiments the therapeutic agent is inosine, rolipram, ATI-355 (NOGO), chondroitinase, fampridine (4-aminopyrideine), Gabapentin, or a Rho antagonist (e.g., Cethrin®). In another embodiment, the therapeutic agent is an immunomodulatory or immunosuppressive agent, e.g., Cyclosporin A, FTY506 (tacrolimus) or FTY720. In other embodiments, the therapeutic agent is a second population of cells that is co-administered with the placental stem cells. In some embodiments, the second population of cells is a population of autologous macrophages, bone marrow stromal cells, nasal olfactory ensheathing cells, embryonic olfactory cortex cells, or Schwann cells.

### 5.1.2 Treatment of Traumatic Brain Injury (TBI)

Also provided herein are methods of treating an individual having, or experiencing, a symptom of, a TBI, comprising administering to the individual a therapeutically effective amount of placental stem cells, or medium conditioned by placental stem cells, wherein the therapeutically effective amount is an amount sufficient to cause a detectable improvement in one or more symptoms of, or a reduction in the progression of one or more symptoms of, said TBI. As used herein, “one or more symptoms” includes objectively measurable parameters, such as degree of inflammation, immune response, gene expression within the site of injury that is correlated with the healing process, quality and extent of scarring at the site of injury, improvement in the patient's motor, sensory and cognitive function, etc., and subjectively measurable parameters, such as patient well-being, patient perception of improvement in motor, sensory and cognitive function, perception of lessening of pain or discomfort associated with the TBI, and the like.

TBI is a nondegenerative, noncongenital insult to the brain from an external mechanical force applied to the cranium and the intracranial contents, possibly leading to permanent or temporary impairment of cognitive, physical, and psychosocial functions, with an associated diminished or altered state of consciousness. TBI can manifest clinically from concussion to coma and death.

In some embodiments of the method of treating TBI, the therapeutically effective amount of placental stem cells is an amount sufficient to cause a detectable improvement in one or more symptoms of primary TBI, i.e., TBI which occurs at the moment of trauma. In some embodiments, the primary TBI is a focal injury, e.g., a skull fracture, a laceration, a contusion, or a penetrating wound. In some embodiments, the primary TBI is diffuse, e.g., diffuse axonal injury.

In some embodiments of the method of treating TBI, the therapeutically effective amount of placental stem cells is an amount sufficient to cause a detectable improvement in one or more symptoms of a secondary injury resulting from primary TBI, which occurs immediately after trauma and produces effects that may continue for some period of time. Secondary types of TBI are attributable to further cellular damage from the effects of primary injuries. Secondary injuries may develop over a period of hours or days following the initial trauma to the brain.

The methods for treating TBI provided herein also encompass the treatment of TBI injuries inflicted upon specific areas to the brain. In some embodiments, the methods of treating TBI provided herein are useful for treating injuries to the frontal lobe (located at the forehead), parietal lobe (located near the back and top of the head), occipital lobe (located most posterior, at the back of the head), temporal lobes (located at the side of head above ears), brain stem (located deep within the brain) and the cerebellum (located at the base of the skull).

In a specific embodiment of the method of treating TBI provided herein, the therapeutically effective amount of placental stem cells (e.g., PDACs) is an amount sufficient to cause an improvement in one or more symptoms of an injury to the frontal lobe, including, but not limited to, loss of simple movement of various body parts (paralysis), inability to plan a sequence of complex movements needed to complete multi-stepped tasks, such as making coffee (sequencing), loss of spontaneity in interacting with others, loss of flexibility in thinking, persistence of a single thought (perseveration), inability to focus on task (attending), mood changes (emotionally labile), changes in social behavior, changes in personality, difficulty with problem solving, or inability to express language (Broca's Aphasia).

In a specific embodiment of the method of treating TBI provided herein, the therapeutically effective amount of placental stem cells (e.g., PDACs) is an amount sufficient to cause an improvement in one or more symptoms of an injury to the parietal lobe, including, but not limited to, an inability to attend to more than one object at a time, an inability to name an object (anomia), an inability to locate the words for writing (agraphia), problems with reading (alexia), difficulty with drawing objects, difficulty in distinguishing left from right, difficulty with doing mathematics (dyscalculia), lack of awareness of certain body parts and/or surrounding space (apraxia) that leads to difficulties in self-care, inability to focus visual attention, or difficulties with eye and hand coordination.

In a specific embodiment of the method of treating TBI provided herein, the therapeutically effective amount of placental stem cells (e.g., PDACs) is an amount sufficient to cause an improvement in one or more symptoms of an injury to the occipital lobe, including, but not limited to, defects in vision (visual field cuts), difficulty with locating objects in environment, difficulty with identifying colors (color agnosia), production of hallucinations, visual illusions (inaccurately seeing objects), word blindness (inability to recognize words), difficulty in recognizing drawn objects, inability to recognize the movement of object (movement agnosia), or difficulties with reading and writing.

In a specific embodiment of the method of treating TBI provided herein, the therapeutically effective amount of placental stem cells (e.g., PDACs) is an amount sufficient to cause an improvement in one or more symptoms of an injury to the temporal lobes including, but not limited to, difficulty in recognizing faces (prosopagnosia), difficulty in understanding spoken words (Wernicke's Aphasia), disturbance with selective attention to what the subject sees and hears, difficulty with identification of, and verbalization about objects, short term memory loss, interference with long term memory, increased and decreased interest in sexual behavior, inability to categorize objects (categorization), persistent talking (indicative of right lobe damage), or increased aggressive behavior.

In a specific embodiment of the method of treating TBI provided herein, the therapeutically effective amount of placental stem cells (e.g., PDACs) is an amount sufficient to cause an improvement in one or more symptoms of an injury to the brain stem, including, but not limited to, decreased vital capacity in breathing (important for speech), difficulty with swallowing food and water (dysphagia), difficulty with organization/perception of the environment, problems with balance and movement, dizziness and nausea (vertigo), or sleeping difficulties (insomnia, sleep apnea).

In a specific embodiment of the method of treating TBI provided herein, the therapeutically effective amount of placental stem cells (e.g., PDACs) is an amount sufficient to cause an improvement in one or more symptoms of an injury to the base of the skull, including, but not limited to, loss of ability to coordinate fine movements, loss of ability to walk, inability to reach out and grab objects, tremors, dizziness (vertigo), slurred speech (scanning speech), or inability to make rapid movements.

The methods for treating TBI provided herein also encompass the treatment of TBI injuries that range in scope from mild to severe. A TBI can be classified as mild if loss of consciousness and/or confusion and disorientation is shorter than 30 minutes. Thus, in some embodiments, the invention provides for the administration of an effective dose of placental stem cells (e.g., PDACS) to an individual affected with a TBI, wherein said effective dose is an amount of placental cell sufficient, e.g., to cause a detectable improvement in, reduce the severity of, or reduce the progression of, one or more symptoms of mild TBI, including, but not limited to, cognitive problems such as headache, memory problems, attention deficits, mood swings and frustration, fatigue, visual disturbances, memory loss, poor attention/concentration, sleep disturbances, dizziness/loss of balance, irritability, emotional disturbances, feelings of depression, seizures, nausea, loss of smell, sensitivity to light and sounds, mood changes, getting lost or confused, or slowness in thinking.

In specific embodiments, the effective dose is an amount of placental cell sufficient, e.g., to cause a detectable improvement in, reduce the severity of, or reduce the progression of, one or more symptoms of a concussion, including, but not limited to, confusion or feeling dazed, clumsiness, slurred speech, nausea or vomiting, headache, balance problems or dizziness, blurred vision, sensitivity to light, sensitivity to noise, sluggishness, ringing in ears, behavior or personality changes, concentration difficulties, or memory loss. In some embodiments, the concussion is a Grade 1 (mild) concussion, characterized by no loss of consciousness and concussion symptoms lasting for less than minutes. In some embodiments, the concussion is a Grade 2 (moderate) concussion, characterized by no loss of consciousness and concussion symptoms lasting for longer than 15 minutes. In some embodiments, the concussion is a Grade 3 (severe) concussion, characterized by a loss of consciousness of at least a few seconds.

In some embodiments, the invention provides for the administration of an effective dose of placental stem cells (e.g., PDACs) to an individual affected with a TBI, wherein said effective dose is an amount of placental stem cells sufficient, e.g., to cause a detectable improvement in, reduce the severity of, or reduce the progression of, one or more symptoms of moderate to severe TBI, including, but not limited to, cognitive deficits such as difficulties with attention, concentration, distractibility, memory, speed of processing, confusion, perseveration, impulsiveness, language processing, speech and language, not understanding the spoken word (receptive aphasia), difficulty speaking and being understood (expressive aphasia), slurred speech, speaking very fast or very slow, problems reading, problems writing; sensory deficits, such as difficulties with interpretation of touch, temperature, movement, limb position or fine discrimination; perceptual deficits, such as difficulty with the integration or patterning of sensory impressions into psychologically meaningful data; visual deficits, including partial or total loss of vision, weakness of eye muscles and double vision (diplopia), blurred vision, problems judging distance, involuntary eye movements (nystagmus), intolerance of light (photophobia); hearing deficits, including a decrease or loss of hearing, or ringing in the ears (tinnitus), or increased sensitivity to sounds; olfactory deficits, including loss or diminished sense of smell (anosmia); loss or diminished sense of taste; seizures, including the convulsions associated with epilepsy that can be several types and can involve disruption in consciousness, sensory perception, or motor movement; physical changes, including physical paralysis/spasticity; chronic pain, loss of control of bowel and/or bladder, sleep disorders, loss of stamina, appetite changes, dysregulation of body temperature, and menstrual difficulties; social-emotional difficulties, including dependent behaviors, lack of emotional ability, lack of motivation, irritability, aggression, depression, disinhibition, or denial/lack of awareness.

In one embodiment, the invention provides for the administration of an effective dose of placental stem cells (e.g., PDACs) to an individual affected with TBI, wherein said effective dose is an amount of placental stem cell sufficient, e.g., to cause a detectable improvement in, reduce the severity of, or reduce the progression of, one or more symptoms of TBI listed above. In certain embodiments, the particular technique(s) for detecting an improvement in, a reduction in the severity of, or a reduction in the progression of, one or more symptoms, conditions, or syndromes of TBI is not critical to the method of treating TBI provided herein. In certain embodiments, the assessment of said improvement or reduction in the progression of one or more symptoms of SCI is determined according to the judgment of a practitioner in the art. In certain embodiments, the assessment of said improvement or reduction in the progression of one or more symptoms of TBI is determined according to the judgment of a practitioner in the art in combination with the subjective experience of the subject.

In some embodiments, an improvement in one or more symptoms of, or a reduction in the progression of one or more symptoms of, said TBI is detected in accordance with the Glasgow Coma Scale (GCS). The GCS defines the severity of a TBI within 48 hours of injury as follows:

Eye Opening

Spontaneous=4

To speech=3

To painful stimulation=2

No response=1

Motor Response

Follows commands=6

Makes localizing movements to pain=5

Makes withdrawal movements to pain=4

Flexor (decorticate) posturing to pain=3

Extensor (decerebrate) posturing to pain=2

No response=1

Verbal Response

Oriented to person, place, and date=5

Converses but is disoriented=4

Says inappropriate words=3

Says incomprehensible sounds=2

No response=1

The severity of TBI according to the GCS score (within 48 h) is as follows:
Vegetative TBI=less than 3 (characterized by sleep wake cycles; arousal, but no interaction with environment; no localized response to pain)
Severe TBI=3-8 (characterized by coma: unconscious state; no meaningful response, no voluntary activities)
Moderate TBI=9-12 (characterized by loss of consciousness greater than 30 minutes; physical or cognitive impairments which may or may resolve; patient may benefit from rehabilitation)
Mild TBI=13-15 (characterized by a brief change in mental status (confusion, disorientation or loss of memory) or loss of consciousness for less than 30 minutes)

Thus, in a specific embodiment of the method of treating TBI provided herein, the therapeutically effective amount of placental stem cells (e.g., PDACs) is an amount sufficient to cause a 1, 2, 3, 4, 5, 6, 7, 8, 9, 10, 11, 12 or higher, point increase in the GCS score of the patient. In some embodiments, the therapeutically effective amount of placental stem cells (e.g., PDACs) is an amount sufficient to cause a 1, 2, or 3 point increase with regard to eye opening, in accordance with the GCS. In some embodiments, the therapeutically effective amount of placental stem cells (e.g., PDACs) is an amount sufficient to cause a 1, 2, 3, 4 or 5 point increase with regard to motor response, in accordance with the GCS. In some embodiments, the therapeutically effective amount of placental stem cells (e.g., PDACs) is an amount sufficient to cause a 1, 2, 3 or 4 point increase with regard to verbal response, in accordance with the GCS. In some embodiments, the therapeutically effective amount of placental stem cells (e.g., PDACs) is an amount sufficient to reduce the severity of the traumatic injury from a level corresponding to vegetative TBI to a level corresponding to severe, moderate or mild TBI. In some embodiments, the therapeutically effective amount of placental stem cells (e.g., PDACs) is an amount sufficient to reduce the severity of the traumatic injury from a level corresponding to severe TBI to a level corresponding to moderate or mild TBI. In some embodiments, the therapeutically effective amount of placental stem cells (e.g., PDACs) is an amount sufficient to reduce the severity of the traumatic injury from a level corresponding to moderate TBI to a level corresponding to mild TBI.

In some embodiments, an improvement in one or more symptoms of, or a reduction in the progression of one or more symptoms of, said TBI is detected in accordance with the Ranchos Los Amigos scale. The Ranchos Los Amigos Scale measures the levels of awareness, cognition, behavior and interaction with the environment, according to the following scale:

Level I: No Response

Level II: Generalized Response

Level III: Localized Response

Level IV: Confused-agitated

Level V: Confused-inappropriate

Level VI: Confused-appropriate

Level VII: Automatic-appropriate

Level VIII: Purposeful-appropriate

Thus, in a specific embodiment of the method of treating TBI provided herein, the therapeutically effective amount of placental stem cells (e.g., PDACs) is an amount sufficient to cause a one, two, three, four, five, six or seven level increase in the score of the patient according to the Rancho Los Amigos Scale. In some embodiments, the therapeutically effective amount of placental stem cells (e.g., PDACs) is an amount sufficient to raise the subject's awareness, cognition, behavior and interaction with the environment from a level of no response to a level of generalized response, localized response, confused agitation, confused inappropriate response, confused appropriate response, automatic appropriate response or purposeful appropriate response. In some embodiments, the therapeutically effective amount of placental stem cells (e.g., PDACs) is an amount sufficient to raise the subject's awareness, cognition, behavior and interaction with the environment from a level of generalized response to a level of localized response, confused agitation, confused inappropriate response, confused appropriate response, automatic appropriate response or purposeful appropriate response. In some embodiments, the therapeutically effective amount of placental stem cells (e.g., PDACs) is an amount sufficient to raise the subject's awareness, cognition, behavior and interaction with the environment from a level of localized response to a level of confused agitation, confused inappropriate response, confused appropriate response, automatic appropriate response or purposeful appropriate response. In some embodiments, the therapeutically effective amount of placental stem cells (e.g., PDACs) is an amount sufficient to raise the subject's awareness, cognition, behavior and interaction with the environment from a level of confused agitation to a level of confused inappropriate response, confused appropriate response, automatic appropriate response or purposeful appropriate response. In some embodiments, the therapeutically effective amount of placental stem cells (e.g., PDACs) is an amount sufficient to raise the subject's awareness, cognition, behavior and interaction with the environment from a level of confused inappropriate response to a level of confused appropriate response, automatic appropriate response or purposeful appropriate response. In some embodiments, the therapeutically effective amount of placental stem cells (e.g., PDACs) is an amount sufficient to raise the subject's awareness, cognition, behavior and interaction with the environment from a level of confused appropriate response to a level of automatic appropriate response or purposeful appropriate response. In some embodiments, the therapeutically effective amount of placental stem cells (e.g., PDACs) is an amount sufficient to raise the subject's awareness, cognition, behavior and interaction with the environment from a level of automatic appropriate response to a level of purposeful appropriate response.

An individual having, or experiencing, a symptom of, TBI, can be treated with a plurality of placental stem cells, and, optionally, one or more therapeutic agents, at any time during the progression of the injury. For example, the individual can be treated immediately after injury, or within 1, 2, 3, 4, 5, 6 days of injury, or within 1, 2, 3, 4, 5, 6, 7, 8, 9, 10, 15, 20, 25, 30, 35, 40, 45, 50 days or more of injury. The individual can be treated once, or multiple times during the clinical course of the injury. In a specific embodiment of the method of treatment, said placental stem cells are administered to said individual within 21 days of development of one or more symptoms of a TBI. In another specific embodiment of the method of treatment, said placental stem cells are administered to said individual within 14 days of development of one or more symptoms of a TBI. In another specific embodiment of the method of treatment, said placental stem cells are administered to said individual within 7 days of development of one or more symptoms of a TBI. In another specific embodiment of the method of treatment, said placental stem cells are administered to said individual within 48 hours of development of one or more symptoms of a TBI. In another specific embodiment, said placental stem cells are administered to said individual within 24 hours of development of one or more symptoms of a TBI. In another specific embodiment, said placental stem cells are administered to said individual within 12 hours of development of one or more symptoms of a TBI. In another specific embodiment, said placental stem cells are administered to said individual within 3 hours of development of one or more symptoms of a TBI.

In certain embodiments of the invention, the individual is an animal, preferably a mammal, more preferably a non-human primate. In certain embodiments, the individual is a human patient. The individual can be a male or female subject. In certain embodiments, the subject is a non-human animal, such as, for instance, a cow, sheep, goat, horse, dog, cat, rabbit, rat or mouse.

The placenta stem cells useful in the treatment of TBI can be any of the placental stem cells disclosed herein (see Section 5.5). In a specific embodiment, the placental stem cells express CD200 and do not express HLA-G; express CD73, CD105, and CD200; express CD200 and OCT-4; express CD73 and CD105 and do not express HLA-G; express CD73 and CD105, and facilitate the formation of one or more embryoid-like bodies in a population of placental stem cells when said population is cultured under conditions that allow for the formation of embryoid-like bodies; or express OCT-4, and (c) facilitate the formation of one or more embryoid-like bodies in a population of placental stem cells when said population is cultured under conditions that allow for the formation of embryoid-like bodies; or any combination of the foregoing. In a specific embodiment, the placental stem cells are CD10⁺, CD105⁺, CD200⁺, CD34⁻ placental stem cells. In another specific embodiment, the placental stem cells are CD117⁻.

In one embodiment, the individual is administered a dose of about 200 million to 800 million placental stem cells. Dosage, however, can vary according to the individual's physical characteristics, e.g., weight, and can range from 1 million to 10 billion placental stem cells per dose, preferably between 10 million and 1 billion per dose, or between 100 million and 500 million placental stem cells per dose. The administration is preferably intravenous, but can be by any medically-acceptable route for the administration of live cells, e.g., intravenous, intraarterial, intraperitoneal, intraventricular, intrasternal, intracranial, intramuscular, intrasynovial, intraocular, intravitreal (e.g., where there is an ocular involvement), intracerebral, intracerebroventricular (e.g., where there is a neurologic or brain involvement), intrathecal, intraosseous infusion, intravesical, transdermal, intracisternal, epidural, or subcutaneous administration. In specific embodiments, administration is by bolus injection or infusion directly into the site of the TBI, e.g., via cisterna magna.

Placental stem cells, or medium conditioned by placental stem cells, can be administered in a single dose, or in multiple doses. Where placental stem cells are administered in multiple doses, the doses can be part of a therapeutic regimen designed to relieve one or more acute symptoms of TBI, or can be part of a long-term therapeutic regimen designed to lessen the severity of TBI.

The methods for treating TBI provided herein further encompass treating TBI by administering a therapeutically effective amount of placental stem cells in conjunction with one or more therapies or treatments used in the course of treating TBI. The one or more additional therapies may be used prior to, concurrent with, or after administration of the placental stem cells. In some embodiments, the one or more additional therapies comprise surgical treatment. In some embodiments, a bolt or ICP (intracranial pressure) monitoring device may be placed in the skull to monitor pressure in the brain cavity. In some embodiments, where there is bleeding in the skull cavity, this may be surgically removed or drained, and bleeding vessels or tissue may be surgically repaired prior to, concurrent with, or after administration of the placental stem cells. In severe cases, if there is extensive swelling and damaged brain tissue, a portion may be surgically removed, to make room for the living brain tissue, prior to, concurrent with, or after administration of the placental stem cells. In some embodiments, the one or more additional therapies comprise the use of mechanical ventilation, which supports breathing and helps keep the pressure down in the head.

Also provided herein are methods for the treatment of an individual having, or experiencing, a symptom of, TBI, comprising administering to the individual a plurality of placental stem cells sufficient to cause a detectable improvement in one or more symptoms, or a reduction in the progression of one or more symptoms of, said TBI, and one or more therapeutic agents. For example, placental stem cells can be administered in conjunction with medications to sedate and put the subject in a drug-induced coma to minimize agitation and secondary injury. In some embodiments, seizure prevention medications may be given early in the course of treatment and later if the individual has seizures. In some embodiments, medications to control spasticity may be used as the patient recovers function. In addition, medications may be used to improve attention and concentration (e.g., amantadine and methylphenidate, bromocriptine and antidepressants), or to control aggressive behavior (e.g., carbamamazapine and amitriptyline).

### 5.2 Use of Placental Stem Cells to Suppress an Inflammatory Response Caused by or Associated with a CNS Injury

In another aspect, provided herein is a method of treating an individual having a CNS injury comprising suppressing an inflammatory response caused by or associated with the CNS injury. Provided herein are methods for the modulation, e.g., suppression, of the activity, e.g., proliferation, of an immune cell, or plurality of immune cells, by contacting the immune cell(s) with a plurality of placental stem cells.

Placental stem cell-mediated immunomodulation, e.g., immunosuppression, would, for example, be advantageous for a CNS injury wherein inflammation plays a role in either or both the early and chronic stages of the CNS injury. In various embodiments, therefore, provided herein is a method of suppressing an immune response, wherein the immune response is caused by or is associated with a CNS injury, e.g., an SCI or TBI.

In one embodiment, provided herein is a method of suppressing an immune response caused by or associated with a CNS injury, e.g., an SCI or TBI, comprising contacting a plurality of immune cells with a plurality of placental stem cells for a time sufficient for said placental stem cells to detectably suppress the immune response, wherein said placental stem cells detectably suppress T cell proliferation in an MLR assay or a regression assay.

Placental stem cells are, e.g., the placental stem cells described elsewhere herein (see Section 5.5). Placental stem cells used for immunosuppression can be derived or obtained from a single placenta or multiple placentas. Placental stem cells used for immunosuppression can also be derived from a single species, e.g., the species of the intended recipient or the species of the immune cells the function of which is to be reduced or suppressed, or can be derived from multiple species.

An “immune cell” in the context of this method means any cell of the immune system, particularly T cells and natural killer (NK) cells. Thus, in various embodiments of the method, placental stem cells are contacted with a plurality of immune cells, wherein the plurality of immune cells are, or comprises, a plurality of T cells (e.g., a plurality of CD3⁺ T cells, CD4⁺ T cells and/or CD8⁺ T cells) and/or natural killer cells. An “immune response” in the context of the method can be any response by an immune cell to a stimulus normally perceived by an immune cell, e.g., a response to the presence of an antigen. In various embodiments, an immune response can be the proliferation of T cells (e.g., CD3⁺ T cells, CD4⁺ T cells and/or CD8⁺ T cells) in response to a CNS injury, e.g., an SCI or TBI. The immune response can also be any activity of a natural killer (NK) cell, the maturation of a dendritic cell, or the like. The immune response can also be a local, tissue- or organ-specific, or systemic effect of an activity of one or more classes of immune cells, e.g., the immune response can be inflammation, formation of inflammation-related scar tissue, and the like.

“Contacting” in this context encompasses bringing the placental stem cells and immune cells together in a single container (e.g., culture dish, flask, vial, etc.) or in vivo, for example, in the same individual (e.g., mammal, for example, human). In a preferred embodiment, the contacting is for a time sufficient, and with a sufficient number of placental stem cells and immune cells, that a change in an immune function of the immune cells is detectable. More preferably, in various embodiments, said contacting is sufficient to suppress immune function (e.g., T cell proliferation in response to an antigen) by at least 50%, 60%, 70%, 80%, 90% or 95%, compared to the immune function in the absence of the placental stem cells. Such suppression in an in vivo context can be determined in an in vitro assay (see below); that is, the degree of suppression in the in vitro assay can be extrapolated, for a particular number of placental stem cells and a number of immune cells in a recipient individual, to a degree of suppression in the individual.

In certain embodiments, provided herein are methods of using placental stem cells to modulate an immune response, or the activity of a plurality of one or more types of immune cells, in vitro. Contacting the placental stem cells and plurality of immune cells can comprise combining the placental stem cells and immune cells in the same physical space such that at least a portion of the plurality of placental stem cells interacts with at least a portion of the plurality of immune cells; maintaining the placental stem cells and immune cells in separate physical spaces with common medium; or can comprise contacting medium from one or a culture of placental stem cells or immune cells with the other type of cell (for example, obtaining culture medium from a culture of placental stem cells and resuspending isolated immune cells in the medium). In a specific example, the contacting is performed in a an MLR assay. In another specific example, the contacting is performed in a regression assay. In another specific example, the contacting is performed in a Bead T-lymphocyte reaction (BTR) assay.

Such contacting can, for example, take place in an experimental setting designed to determine the extent to which a particular plurality of placental stem cells is immunomodulatory, e.g., immunosuppressive. Such an experimental setting can be, for example, an MLR or regression assay. Procedures for performing the MLR and regression assays are well-known in the art. See, e.g. Schwarz, “The Mixed Lymphocyte Reaction: An In Vitro Test for Tolerance,” J. Exp. Med. 127(5):879-890 (1968); Lacerda et al., “Human Epstein-Barr Virus (EBV)-Specific Cytotoxic T Lymphocytes Home Preferentially to and Induce Selective Regressions of Autologous EBV-Induced B Lymphoproliferations in Xenografted C.B-17 Scid/Scid Mice,” J. Exp. Med. 183:1215-1228 (1996). In a preferred embodiment, an MLR is performed in which pluralities of placental stem cells are contacted with a plurality of immune cells (e.g., lymphocytes, for example, CD3⁺, CD4⁺ and/or CD8⁺ T lymphocytes).

The MLR can be used to determine the immunosuppressive capacity of a plurality of placental stem cells. For example, a plurality of placental stem cells can be tested in an MLR comprising combining CD4⁺ or CD8⁺ T cells, dendritic cells (DC) and placental stem cells in a ratio of about 10:1:2, wherein the T cells are stained with a dye such as, e.g., CFSE that partitions into daughter cells, and wherein the T cells are allowed to proliferate for about 6 days. The plurality of placental stem cells is immunosuppressive if the T cell proliferation at 6 days in the presence of placental stem cells is detectably reduced compared to T cell proliferation in the presence of DC and absence of placental stem cells. In such an MLR, placental stem cells are either thawed or harvested from culture. About 20,000 placental stem cells are resuspended in 100 μl of medium (RPMI 1640, 1 mM HEPES buffer, antibiotics, and 5% pooled human serum), and allowed to attach to the bottom of a well for 2 hours. CD4⁺ and/or CD8⁺ T cells are isolated from whole peripheral blood mononuclear cells Miltenyi magnetic beads. The cells are CFSE stained, and a total of 100,000 T cells (CD4⁺ T cells alone, CD8⁺ T cells alone, or equal amounts of CD4⁺ and CD8⁺ T cells) are added per well. The volume in the well is brought to 200 μl, and the MLR is allowed to proceed.

In one embodiment, therefore, provided herein is a method of suppressing an immune response comprising contacting a plurality of immune cells with a plurality of placental stem cells for a time sufficient for said placental stem cells to detectably suppress T cell proliferation in an MLR assay or in a regression assay. In one embodiment, said placental stem cells used in the MLR represent a sample or aliquot of placental stem cells from a larger population of placental stem cells.

Populations of placental stem cells obtained from different placentas, or different tissues within the same placenta, can differ in their ability to modulate an activity of an immune cell, e.g., can differ in their ability to suppress T cell activity or proliferation or NK cell activity. It is thus desirable to determine, prior to use, the capacity of a particular population of placental stem cells for immunosuppression. Such a capacity can be determined, for example, by testing a sample of the placental stem cell population in an MLR or regression assay. In one embodiment, an MLR is performed with the sample, and a degree of immunosuppression in the assay attributable to the placental stem cells is determined. This degree of immunosuppression can then be attributed to the placental stem cell population that was sampled. Thus, the MLR can be used as a method of determining the absolute and relative ability of a particular population of placental stem cells to suppress immune function. The parameters of the MLR can be varied to provide more data or to best determine the capacity of a sample of placental stem cells to immunosuppress. For example, because immunosuppression by placental stem cells appears to increase roughly in proportion to the number of placental stem cells present in the assay, the MLR can be performed with, in one embodiment, two or more numbers of placental stem cells, e.g., 1×10³, 3×10³, 1×10⁴ and/or 3×10⁴ placental stem cells per reaction. The number of placental stem cells relative to the number of T cells in the assay can also be varied. For example, placental stem cells and T cells in the assay can be present in any ratio of, e.g. about 10:1 to about 1:10, preferably about 1:5, though a relatively greater number of placental stem cells or T cells can be used.

The regression assay or BTR assay can be used in similar fashion.

Provided herein are methods of using placental stem cells to modulate an immune response, or the activity of a plurality of one or more types of immune cells, in vivo, for example, caused by or associated with a CNS injury, e.g., an SCI or TBI. Placental stem cells and immune cells can be contacted, e.g., in an individual that is a recipient of a plurality of placental stem cells. Where the contacting is performed in an individual, in one embodiment, the contacting is between exogenous placental stem cells (that is, placental stem cells not derived from the individual) and a plurality of immune cells endogenous to the individual. In specific embodiments, the immune cells within the individual are CD3⁺ T cells, CD4⁺ T cells, CD8⁺ T cells, and/or NK cells.

The placental stem cells can be administered to the individual in a ratio, with respect to the known or expected number of immune cells, e.g., T cells, in the individual, of from about 10:1 to about 1:10, preferably about 1:5. However, a plurality of placental stem cells can be administered to an individual in a ratio of in non-limiting examples, about 10,000:1, about 1,000:1, about 100:1, about 10:1, about 1:1, about 1:10, about 1:100, about 1:1,000 or about 1:10,000. Generally, about 1×10⁵ to about 1×10⁸ placental stem cells per recipient kilogram, preferably about 1×10⁶ to about 1×10⁷ placental stem per recipient kilogram can be administered to effect immunosuppression. In various embodiments, a plurality of placental stem cells administered to an individual or subject comprises at least, about, or no more than, 1×10⁵, 3×10⁵, 1×10⁶, 3×10⁶, 1×10⁷, 3×10⁷, 1×10⁸, 3×10⁸, 1×10⁹, 3×10⁹ placental stem cells, or more.

The placental stem cells can also be administered with one or more second types of stem cells, e.g., mesenchymal stem cells from bone marrow. Such second stem cells can be administered to an individual with placental stem cells in a ratio of, e.g., about 1:10 to about 10:1.

To facilitate contacting, or proximity of, the placental stem cells and immune cells in vivo, the placental stem cells can be administered to the individual by any route sufficient to bring the placental stem cells and immune cells into contact with each other. For example, the placental stem cells can be administered to the individual, e.g., intravenously, intramuscularly, intraperitoneally, intraocularly, parenterally, intrathecally, or directly into an organ, e.g., pancreas. For in vivo administration, the placental stem cells can be formulated as a pharmaceutical composition, as described in Section 5.9.1.2, below.

The method of immunosuppression can additionally comprise the addition of one or more immunosuppressive agents, particularly in the in vivo context. In one embodiment, the plurality of placental stem cells are contacted with the plurality of immune cells in vivo in an individual, and a composition comprising an immunosuppressive agent is administered to the individual. Immunosuppressive agents are well-known in the art and include, e.g., anti-T cell receptor antibodies (monoclonal or polyclonal, or antibody fragments or derivatives thereof), anti-IL-2 receptor antibodies (e.g., Basiliximab (SIMULECT®) or daclizumab (ZENAPAX)®), anti T cell receptor antibodies (e.g., Muromonab-CD3), azathioprine, corticosteroids, cyclosporine, tacrolimus, mycophenolate mofetil, sirolimus, calcineurin inhibitors, and the like. In a specific embodiment, the immumosuppressive agent is a neutralizing antibody to macrophage inflammatory protein (MIP)-1α or MIP-1β. Preferably, the anti-MIP-1α or MIP-1β antibody is administered in an amount sufficient to cause a detectable reduction in the amount of MIP-1α and/or MIP-1β in said individual.

Placental stem cells, in addition to suppression of proliferation of T cells, have other anti-inflammatory effects on cells of the immune system which can be beneficial in the treatment of a CNS injury, e.g., an SCI or TBI. For example, placental stem cells, e.g., in vitro or in vivo, as when administered to an individual, reduce an immune response mediated by a Th1 and/or a Th17 T cell subset. In another aspect, provided herein is a method of inhibiting a pro-inflammatory response, e.g., a Th1 response or a Th17 response, either in vivo or in vitro, comprising contacting T cells (e.g., CD4⁺ T lymphocytes or leukocytes) with placental stem cells, e.g., the placental stem cells described herein. In a specific embodiment, said contacting detectably reduces Th1 cell maturation. In a specific embodiment of the method, said contacting detectably reduces the production of one or more of lymphotoxin-1α (LT-1α), interleukin-1β (IL-1β), IL-12, IL-17, IL-21, IL-23, tumor necrosis factor alpha (TNFα) and/or interferon gamma (IFNγ) by said T cells or by an antigen-producing cell. In another specific embodiment of the method, said contacting potentiates or upregulates a regulatory T cell (Treg) phenotype, and/or reduces expression in a dendritic cell (DC) and/or macrophage of biomolecules that promote a Th1 and/or Th17 response (e.g., CD80, CD83, CD86, ICAM-1, HLA-II). In a specific embodiment, said T cells are also contacted with IL-10, e.g., exogenous IL-10 or IL-10 not produced by said T cells, e.g., recombinant IL-10. In another embodiment, provided herein is a method of reducing the production of pro-inflammatory cytokines from macrophages, comprising contacting the macrophages with an effective amount of placental stem cells. In another embodiment, provided herein is a method of increasing a number of tolerogenic cells, promoting tolerogenic functions of immune cells, and/or upragulating tolerogenic cytokines, e.g., from macrophages, comprising contacting immune system cells with an effective amount of placental stem cells. In a specific embodiment, said contacting causes activated macrophages to produce detectably more IL-10 than activated macrophages not contacted with said placental stem cells. In another embodiment, provided herein is a method of upregulating, or increasing the number of, anti-inflammatory T cells, comprising contacting immune system cells with an effective amount of placental stem cells.

In one embodiment, provided herein is a method of inhibiting a Th1 response in an individual having, or experiencing, a symptom of, a CNS injury, e.g., an SCI or TBI, comprising administering to the individual an effective amount of placental stem cells, wherein said effective amount is an amount that results in a detectable decrease in a Th1 response in the individual. In another embodiment, provided herein is a method of inhibiting a Th17 response in an individual having, or experiencing, a symptom of, a CNS injury, e.g., an SCI or TBI, comprising administering to the individual an effective amount of placental stem cells, wherein said effective amount is an amount that results in a detectable decrease in a Th17 response in the individual. In specific embodiments of these methods, said administering detectably reduces the production, by T cells or antigen presenting cells in said individual, of one or more of IL-1β, IL-12, IL-17, IL-21, IL-23, TNFα and/or IFNγ. In another specific embodiment of the method, said contacting potentiates or upregulates a regulatory T cell (Treg) phenotype, or modulates production in a dendritic cell (DC) and/or macrophage in said individual of markers the promote a Th1 or Th17 response. In another specific embodiment, the method comprises additionally administering IL-10 to said individual.

In another aspect, provided herein are placental stem cells, as described herein, that have been genetically engineered to express one or more anti-inflammatory cytokines. In a specific embodiment, said anti-inflammatory cytokines comprise IL-10.

### 5.3 Angiogenesis and Treatment of CNS Injuries

In another aspect, provided herein is a method of treating an individual who has a disruption of the flow of blood in or around the individual's brain, e.g., who has a symptom or neurological deficit attributable to a disruption of the flow of blood in or around the individual's brain or CNS resulting from a CNS injury, e.g., an SCI or TBI, comprising administering to said individual a therapeutically effective amount of isolated placental stem cells (e.g., PDACs). In certain embodiments, the disruption of flow of blood results in anoxic injury or hypoxic injury to the individual's brain or CNS. In certain embodiments, the PDACs are angiogenic. In certain embodiments, the PDACs are able to support growth of endothelial cells and endothelial cells populations, and epithelial cells and epithelial cell populations, both in vitro and in vivo.

As used herein, the term “angiogenic,” in reference to the placental derived adherent cells described herein, means that the cells can form vessels or vessel-like sprouts, or that the cells can promote angiogenesis (e.g., the formation of vessels or vessel-like structures) in another population of cells, e.g., endothelial cells.

As used herein, the term “angiogenesis” refers to the process of blood vessel formation that includes, but is not limited to, endothelial cell activation, migration, proliferation, matrix remodeling and cell stabilization.

The PDACs, and populations of such cells, can, in certain embodiments, be used to promote angiogenesis in individuals exhibiting traumatic tissue loss, or to prevent scar formation, resulting from a CNS injury, e.g., an SCI or TBI.

In certain embodiments, the individual experiences benefits from the therapy, for example from the ability of the cells to support the growth of other cells, including oligodendrocytes and neurons, from the tissue ingrowth or vascularization of the tissue, and from the presence of beneficial cellular factors, chemokines, cytokines and the like, but the cells do not integrate or multiply in the individual. In another embodiment, the patient benefits from the therapeutic treatment with the cells, but the cells do not survive for a prolonged period in the patient. In one embodiment, the cells gradually decline in number, viability or biochemical activity, in other embodiments, the decline in cells may be preceded by a period of activity, for example growth, division, or biochemical activity. In other embodiments, senescent, nonviable or even dead cells are able to have a beneficial therapeutic effect.

Administration of PDACs, or therapeutic compositions comprising such cells, to an individual in need thereof, can be accomplished, e.g., by transplantation, implantation (e.g., of the cells themselves or the cells as part of a matrix-cell combination), injection (e.g., directly to the site of the CNS injury, infusion, delivery via catheter, or any other means known in the art for providing cell therapy.

To this end, further provided herein are populations of PDACs contacted with, e.g., incubated or cultured in the presence of, one or more factors that stimulate stem or progenitor cell differentiation along a angiogenic, hemangiogenic, or vasculogenic pathway. Such factors include, but are not limited to factors, such as growth factors, chemokines, cytokines, cellular products, demethylating agents, and other factors which are now known or later determined to stimulate differentiation, for example of stem cells, along angiogenic, hemangiogenic, or vasculogenic pathways or lineages.

In certain embodiments, PDACs may be differentiated along angiogenic, hemangiogenic, or vasculogenic pathways or lineages in vitro by culture of the cells in the presence of factors comprising at least one of a demethylation agent, a BMP (bone morphogenetic protein), FGF (fibroblast growth factor), Wnt factor protein, Hedgehog protein, and/or an anti-Wnt factor.

The PDACs may be administered to an individual in the form of a therapeutic composition comprising the cells and another therapeutic agent, such as insulin-like growth factor (IGF), platelet-derived growth factor (PDGF), epidermal growth factor (EGF), fibroblast growth factor (FGF), vascular endothelial growth factor (VEGF), hepatocyte growth factor (HGF), interleukin 18 (IL-8), an antithrombogenic agent (e.g., heparin, heparin derivatives, urokinase, or PPack (dextrophenylalanine proline arginine chloromethylketone), an antithrombin compound, a platelet receptor antagonist, an anti-thrombin antibody, an anti-platelet receptor antibody, aspirin, dipyridamole, protamine, hirudin, a prostaglandin inhibitor, and/or a platelet inhibitor), an antiapoptotic agent (e.g., erythropoietin (Epo), an Epo derivative or analog, or their salts, thrombopoietin (Tpo), IGF-I, IGF-II, hepatocyte growth factor (HGF), or a caspase inhibitor), an anti-inflammatory agent (e.g., a p38 MAP kinase inhibitor, a statin, in IL-6 inhibitor, an IL-1 inhibitor, Pemirolast, Tranilast, Remicade, Sirolimus, and/or a nonsteroidal anti-inflammatory compound (e.g., acetylsalicylic acid, ibuprofen, Tepoxalin, Tolmetin, or Suprofen)), an immunosuppressive or immunomodulatory agent (e.g., a calcineurin inhibitor, for example cyclosporine, Tacrolimus, an mTOR inhibitor such as Sirolimus or Everolimus; an anti-proliferative such as azathioprine and/or mycophenolate mofetil; a corticosteroid, e.g., prednisolone or hydrocortisone; an antibody such as a monoclonal anti-IL-2Rα receptor antibody, Basiliximab, Daclizuma, polyclonal anti-T-cell antibodies such as anti-thymocyte globulin (ATG), anti-lymphocyte globulin (ALG), and/or the monoclonal anti-T cell antibody OKT3, or adherent placental stem cells as described in U.S. Pat. No. 7,468,276, and U.S. Patent Application Publication No. 2007/0275362, the disclosures of each of which are incorporated herein by reference in their entireties), and/or an antioxidant (e.g., probucol; vitamins A, C, and/or E, coenzyme Q-10, glutathione, L cysteine, N-acetylcysteine, or an antioxidant derivative, analog or salt of any of the foregoing). In certain embodiments, therapeutic compositions comprising the PDACs further comprise one or more additional cell types, e.g. adult cells (for example, fibroblasts or endodermal cells), stem cells and/or progenitor cells. Such therapeutic agents and/or one or more additional types of cells can be administered to an individual in need thereof individually or in combinations or two or more such compounds or agents.

### 5.4 Second Therapeutic Compositions and Second Therapies

In any of the above methods of treatment, the method can comprise the administration of a second therapeutic composition or second therapy. The recitation of specific second therapeutic compounds or second therapies in the methods of treating specific diseases, above, are not intended to be exclusive. For example, any of the diseases, disorders or conditions discussed herein can be treated with any of the anti-inflammatory compounds or immunosuppressive compounds described herein. In embodiments in which placental stem cells are administered with a second therapeutic agent, or with a second type of stem cell, the placental stem cells and second therapeutic agent and/or second type of stem cell can be administered at the same time or different times, e.g., the administrations can take place within 1, 2, 3, 4, 5, 6, 7, 8, 9 10, 20, 30, 40, or 50 minutes of each other, or 1, 2, 3, 4, 5, 6, 7, 8, 9, 10, 12, 14, 16, 18, 20, or 22 hours of each other, or within 1, 2, 3, 4, 5, 6, 7 8, 9 or 10 days of each other.

In a specific embodiment, treatment of a disease, disorder or condition related to or caused by an inappropriate, deleterious or harmful immune response comprises administration of a second type of stem cell, or population of a second type of stem cell. In a specific embodiment, said second type of stem cell is a mesenchymal stem cell, e.g., a bone marrow-derived mesenchymal stem cell. In other embodiments, the second type of stem cell is a multipotent stem cell, a pluripotent stem cell, a progenitor cell, a hematopoietic stem cell, e.g., a CD34⁺ hematopoietic stem cell, an adult stem cell, an embryonic stem cell or an embryonic germ cell. The second type of stem cell, e.g., mesenchymal stem cell, can be administered with the placental stem cells in any ratio, e.g., a ratio of about 100:1, 75:1, 50:1, 25:1, 20:1, 15:1, 10:1, 5:1, 1:1, 1:5, 1:10, 1:15, 1:20, 1:25, 1:50, 1:75 or 1:100. Mesenchymal stem cells can be obtained commercially or from an original source, e.g., bone marrow, bone marrow aspirate, adipose tissue, and the like.

In another specific embodiment, said second therapy comprises an immunomodulatory compound, wherein the immunomodulatory compound is 3-(4-amino-1-oxo-1,3-dihydroisoindol-2-yl)-piperidine-2,6-dione; 3-(4′aminoisolindoline-1′-onw)-1-piperidine-2,6-dione; 4-(Amino)-2-(2,6-dioxo(3-piperidyl))-isoindoline-1,3-dione; or α-(3-aminophthalimido) glutarimide. In a more specific embodiment, said immunomodulatory compound is a compound having the structure

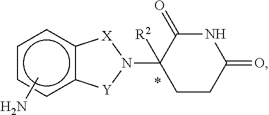

wherein one of X and Y is C═O, the other of X and Y is C═O or CH₂, and R² is hydrogen or lower alkyl, or a pharmaceutically acceptable salt, hydrate, solvate, clathrate, enantiomer, diastereomer, racemate, or mixture of stereoisomers thereof. In another more specific embodiment, said immunomodulatory compound is a compound having the structure

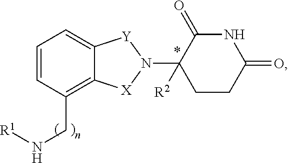

wherein one of X and Y is C═O and the other is CH₂ or C═O;

R¹ is H, (C₁-C₈)alkyl, (C₃-C₇)cycloalkyl, (C₂-C₈)alkenyl, (C₂-C₈)alkynyl, benzyl, aryl, (C₀-C₄)alkyl-(C₁-C₆)heterocycloalkyl, (C₀-C₄)alkyl-(C₂-C₅)heteroaryl, C(O)R³, C(S)R³, C(O)OR⁴, (C₁-C₈)alkyl-N(R⁶)₂, (C₁-C₈)alkyl-OR⁵, (C₁-C₈)alkyl-C(O)OR^{S}, C(O)NHR³, C(S)NHR³, C(O)NR³R^{3′}, C(S)NR³R^{3′} or (C₁-C₈)alkyl-O(CO)R⁵;

R² is H, F, benzyl, (C₁-C₈)alkyl, (C₂-C₈)alkenyl, or (C₂-C₈)alkynyl;

R³ and R^{3′} are independently (C₁-C₈)alkyl, (C₃-C₇)cycloalkyl, (C₂-C₈)alkenyl, (C₂-C₈)alkynyl, benzyl, aryl, (C₀-C₄)alkyl-(C₁-C₆)heterocycloalkyl, (C₀-C₄)alkyl-(C₂-C₅)heteroaryl, (C₀-C₈)alkyl-N(R⁶)₂, (C₁-C₈)alkyl-OR⁵, (C₁-C₈)alkyl-C(O)OR^{S}, (C₁-C₈)alkyl-O(CO)R⁵, or C(O)OR⁵;

R⁴ is (C₁-C₈)alkyl, (C₂-C₈)alkenyl, (C₂-C₈)alkynyl, (C₁-C₄)alkyl-OR⁵, benzyl, aryl, (C₀-C₄)alkyl-(C₁-C₆)heterocycloalkyl, or (C₀-C₄)alkyl-(C₂-C₅)heteroaryl;

R⁵ is (C₁-C₈)alkyl, (C₂-C₈)alkenyl, (C₂-C₈)alkynyl, benzyl, aryl, or (C₂-C₈)heteroaryl;

each occurrence of R⁶ is independently H, (C₁-C₈)alkyl, (C₂-C₈)alkenyl, (C₂-C₈)alkynyl, benzyl, aryl, (C₂-C₅)heteroaryl, or (C₀-C₈)alkyl-C(O)O—R⁵ or the R⁶ groups can join to form a heterocycloalkyl group;

n is 0 or 1; and

* represents a chiral-carbon center;

or a pharmaceutically acceptable salt, hydrate, solvate, clathrate, enantiomer, diastereomer, racemate, or mixture of stereoisomers thereof. In another more specific embodiment, said immunomodulatory compound is a compound having the structure

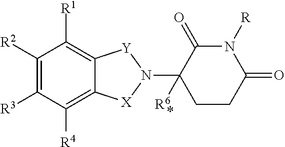

wherein:

one of X and Y is C═O and the other is CH₂ or C═O;

R is H or CH₂OCOR′;

(i) each of R¹, R², R³, or R⁴, independently of the others, is halo, alkyl of 1 to 4 carbon atoms, or alkoxy of 1 to 4 carbon atoms or (ii) one of R¹, R², R³, or R⁴ is nitro or —NHR⁵ and the remaining of R¹, R², R³, or R⁴ are hydrogen;

R⁵ is hydrogen or alkyl of 1 to 8 carbons

R⁶ hydrogen, alkyl of 1 to 8 carbon atoms, benzo, chloro, or fluoro;

R′ is R⁷—CHR¹⁹—N(R⁸R⁹);

R⁷ is m-phenylene or p-phenylene or —(CₙH₂ₙ)— in which n has a value of 0 to 4;

each of R⁸ and R⁹ taken independently of the other is hydrogen or alkyl of 1 to 8 carbon atoms, or R⁸ and R⁹ taken together are tetramethylene, pentamethylene, hexamethylene, or —CH₂CH₂X₁CH₂CH₂— in which X₁ is —O—, —S—, or —NH—;

R¹⁰ is hydrogen, alkyl of to 8 carbon atoms, or phenyl; and

represents a chiral-carbon center;

or a pharmaceutically acceptable salt, hydrate, solvate, clathrate, enantiomer, diastereomer, racemate, or mixture of stereoisomers thereof.

Any combination of the above therapeutic agents can be administered. Such therapeutic agents can be administered in any combination with the placental stem cells, at the same time or as a separate course of treatment.

Placental stem cells can be administered to the individual suffering from a CNS injury, e.g., an SCI or TBI, in the form of a pharmaceutical composition, e.g., a pharmaceutical composition suitable for intravenous, intramuscular or intraperitoneal injection. Placental stem cells can be administered in a single dose, or in multiple doses. Where placental stem cells are administered in multiple doses, the doses can be part of a therapeutic regimen designed to relieve one or more acute symptoms of CNS injury, e.g., an SCI or TBI, or can be part of a long-term therapeutic regimen designed to prevent, or lessen the severity, of a chronic course of the injury. In embodiments in which placental stem cells are administered with a second therapeutic agent, or with a second type of stem cell, the placental stem cells and second therapeutic agent and/or second type of stem cell can be administered at the same time or different times, e.g., the administrations can take place within 1, 2, 3, 4, 5, 6, 7, 8, 9 10, 20, 30, 40, or 50 minutes of each other, or 1, 2, 3, 4, 5, 6, 7, 8, 9, 10, 12, 14, 16, 18, 20, or 22 hours of each other, or within 1, 2, 3, 4, 5, 6, 7 8, 9 or 10 days or more of each other.

### 5.5 Placental Stem Cells and Placental Stem Cell Populations

The methods provided herein use placental stem cells, that is, stem cells obtainable from a placenta or part thereof, that (1) adhere to a tissue culture substrate; (2) have the capacity to differentiate into non-placental cell types; and (3) have, in sufficient numbers, the capacity to detectably suppress an immune function, e.g., proliferation of CD4⁺ and/or CD8⁺ T cells in an MLR assay or regression assay. Placental stem cells are not derived from blood, e.g., placental blood or umbilical cord blood. The placental stem cells used in the methods and compositions provided herein have the capacity, and are selected for their capacity, to suppress the immune system of an individual.

Placental stem cells can be either fetal or maternal in origin (that is, can have the genotype of either the mother or fetus). Populations of placental stem cells, or populations of cells comprising placental stem cells, can comprise placental stem cells that are solely fetal or maternal in origin, or can comprise a mixed population of placental stem cells of both fetal and maternal origin. The placental stem cells, and populations of cells comprising the placental stem cells, can be identified and selected by the morphological, marker, and culture characteristics discussed below.

### 5.5.1 Physical and Morphological Characteristics

The placental stem cells used as described herein, when cultured in primary cultures or in cell culture, adhere to the tissue culture substrate, e.g., tissue culture container surface (e.g., tissue culture plastic). Placental stem cells in culture assume a generally fibroblastoid, stellate appearance, with a number of cyotplasmic processes extending from the central cell body. The placental stem cells are, however, morphologically differentiable from fibroblasts cultured under the same conditions, as the placental stem cells exhibit a greater number of such processes than do fibroblasts. Morphologically, placental stem cells are also differentiable from hematopoietic stem cells, which generally assume a more rounded, or cobblestone, morphology in culture.

### 5.5.2 Cell Surface, Molecular and Genetic Markers

The isolated placental stem cells, e.g., isolated multipotent placental stem cells or isolated placental stem cells, and populations of such isolated placental stem cells, useful in the methods disclosed herein, e.g., the methods of treatment of a CNS injury, are tissue culture plastic-adherent human placental stem cells that have characteristics of multipotent cells or stem cells, and express a plurality of markers that can be used to identify and/or isolate the cells, or populations of cells that comprise the stem cells. In certain embodiments, the PDACs are angiogenic, e.g., in vitro or in vivo. The isolated placental stem cells, and placental cell populations described herein (that is, two or more isolated placental stem cells) include placental stem cells and placental cell-containing cell populations obtained directly from the placenta, or any part thereof (e.g., chorion, placental cotyledons, or the like). Isolated placental cell populations also include populations of (that is, two or more) isolated placental stem cells in culture, and a population in a container, e.g., a bag. The isolated placental stem cells described herein are not bone marrow-derived mesenchymal cells, adipose-derived mesenchymal stem cells, or mesenchymal cells obtained from umbilical cord blood, placental blood, or peripheral blood. Placental cells, e.g., placental multipotent cells and placental stem cells, useful in the methods and compositions described herein are described herein and, e.g., in U.S. Pat. Nos. 7,311,904; 7,311,905; and 7,468,276; and in U.S. Patent Application Publication No. 2007/0275362, the disclosures of which are hereby incorporated by reference in their entireties.

In certain embodiments, the isolated placental cells are isolated placental stem cells. In certain other embodiments, the isolated placental cells are isolated placental multipotent cells. In one embodiment, the isolated placental cells, e.g., PDACs, are CD34⁻, CD10⁺ and CD105⁺ as detected by flow cytometry. In another specific embodiment, the isolated CD34⁻. CD10⁺, CD105⁺ placental cells have the potential to differentiate into cells of a neural phenotype, cells of an osteogenic phenotype, and/or cells of a chondrogenic phenotype. In another specific embodiment, the isolated CD34⁻, CD10⁺, CD105⁺ placental cells are additionally CD200⁺. In another specific embodiment, the isolated CD34⁻, CD10⁺. CD105⁺ placental cells are additionally CD45⁻ or CD90⁺. In another specific embodiment, the isolated CD34⁻, CD10⁺, CD105⁺ placental cells are additionally CD45⁻ and CD90⁺, as detected by flow cytometry. In another specific embodiment, the isolated CD34⁻, CD10⁺, CD105⁺, CD200⁺ placental cells are additionally CD90⁺ or CD45⁻, as detected by flow cytometry. In another specific embodiment, the isolated CD34⁻, CD10⁺, CD105⁺, CD200⁺ placental cells are additionally CD90⁺ and CD45⁻, as detected by flow cytometry, i.e., the cells are CD34⁻, CD10⁺, CD45⁻, CD90⁺, CD105⁺ and CD200⁺. In another specific embodiment, said CD34⁻, CD10⁺, CD45⁻, CD90⁺, CD105⁺, CD200⁺ cells are additionally CD80⁻ and CD86⁻.

In certain embodiments, said placental cells are CD34⁻, CD10⁺, CD105⁺ and CD200⁺, and one or more of CD38⁻, CD45⁻, CD80⁻, CD86⁻, CD133⁻, HLA-DR,DP,DQ⁻, SSEA3⁻, SSEA4⁻, CD29⁺, CD44⁺, CD73⁺, CD90⁺, CD105⁺, HLA-A,B,C⁺, PDL1⁺, ABC-p⁺, and/or OCT-4⁺, as detected by flow cytometry. In other embodiments, any of the CD34⁻, CD10⁺, CD105⁺ cells described above are additionally one or more of CD29⁺, CD38⁻, CD44⁺, CD54⁺, SH3⁺ or SH4⁺. In another specific embodiment, the cells are additionally CD44⁺. In another specific embodiment of any of the isolated CD34⁻, CD10⁺, CD105⁺ placental cells above, the cells are additionally one or more of CD117⁻, CD133⁻, KDR⁻ (VEGFR2⁻), HLA-A,B,C⁺, HLA-DP,DQ,DR⁻, or Programmed Death-1 Ligand (PDL1)⁺, or any combination thereof.

In another embodiment, the CD34⁻, CD10⁺, CD105⁺ cells are additionally one or more of CD13⁺, CD29⁺, CD33⁺, CD38⁻, CD44⁺, CD45⁻, CD54⁺, CD62E⁻, CD62L⁻, CD62P⁻, SH3⁺ (CD73⁺), SH4⁺ (CD73⁺), CD80⁻, CD86⁻, CD90⁺, SH2⁺ (CD105″), CD106/VCAM⁺, CD117⁻, CD144/VE-cadherin^{low}, CD184/CXCR4⁻, CD200⁺, CD133⁻, OCT-4⁺, SSEA3⁻, SSEA4⁻, ABC-p⁺, KDR⁻ (VEGFR2⁻), HLA-A,B,C⁺, HLA-DP,DQ,DR⁻, HLA-G⁻, or Programmed Death-1 Ligand (PDL1)⁺, or any combination thereof. In another embodiment, the CD34⁻, CD10⁺, CD105⁺ cells are additionally CD13⁺, CD29⁺, CD33⁺, CD38⁻, CD44⁺, CD45⁻, CD54/ICAM⁺, CD62E⁻, CD62L⁻, CD62P⁻, SH3⁺ (CD73⁺), SH4⁺ (CD73⁺), CD80⁻, CD86⁻, CD90⁺, SH2⁺ (CD105⁺), CD106/VCAM⁺, CD117⁻, CD144/VE-cadherin^{low}, CD184/CXCR4⁻, CD200⁺, CD133⁻, OCT-4″, SSEA3⁻, SSEA4⁻, ABC-p⁺, KDR⁻ (VEGFR2⁻), HLA-A,B,C⁺, HLA-DP,DQ,DR⁻, HLA-G⁻, and Programmed Death-1 Ligand (PDL1)⁺.

In another specific embodiment, any of the placental cells described herein are additionally ABC-p⁺, as detected by flow cytometry, or OCT-4⁺ (POU5F1), as determined by reverse-transcriptase polymerase chain reaction (RT-PCR), wherein ABC-p is a placenta-specific ABC transporter protein (also known as breast cancer resistance protein (BCRP) and as mitoxantrone resistance protein (MXR)), and OCT-4 is the Octamer-4 protein (POU5F1). In another specific embodiment, any of the placental cells described herein are additionally SSEA3⁻ or SSEA4⁻, as determined by flow cytometry, wherein SSEA3 is Stage Specific Embryonic Antigen 3, and SSEA4 is Stage Specific Embryonic Antigen 4. In another specific embodiment, any of the placental cells described herein are additionally SSEA3⁻ and SSEA4⁻.

In another specific embodiment, any of the placental cells described herein are additionally one or more of MHC-I⁺ (e.g., HLA-A,B,C⁺), MHC-II⁻ (e.g., HLA-DP,DQ,DR⁻) or HLA-G⁻. In another specific embodiment, any of the placental cells described herein are additionally one or more of MHC-I⁺ (e.g., HLA-A,B,C⁺), MHC-IV (e.g., HLA-DP,DQ,DR⁻) and HLA-G⁻.

Also provided herein are populations of the isolated placental cells, or populations of cells, e.g., populations of placental cells, comprising, e.g., that are enriched for, the isolated placental cells, that are useful in the methods and compositions disclosed herein. Preferred populations of cells comprising the isolated placental cells, wherein the populations of cells comprise, e.g., at least 10%, 15%, 20%, 25%, 30%, 35%, 40%, 45%, 50%, 55%, 60%, 65%, 70%, 75%, 80%, 85%, 90%, 95% or 98% isolated CD10⁺, CD105⁺ and CD34⁻ placental cells; that is, at least 10%, 15%, 20%, 25%, 30%, 35%, 40%, 45%, 50%, 55%, 60%, 65%, 70%, 75%, 80%, 85%, 90%, 95% or 98% of cells in said population are isolated CD10⁺, CD105⁺ and CD34⁻ placental cells. In a specific embodiment, the isolated CD34⁻, CD10⁺, CD105⁺ placental cells are additionally CD200⁺. In another specific embodiment, the isolated CD34⁻, CD10⁺, CD105⁺, CD200⁺ placental cells are additionally CD90⁺ or CD45⁻, as detected by flow cytometry. In another specific embodiment, the isolated CD34⁻, CD10⁺, CD105⁺, CD200⁺ placental cells are additionally CD90⁺ and CD45⁻, as detected by flow cytometry. In another specific embodiment, any of the isolated CD34⁻, CD10⁺, CD105⁺ placental cells described above are additionally one or more of CD29⁺, CD38⁻, CD44⁺, CD54⁺, SH3⁺ or SH4⁺. In another specific embodiment, the isolated CD34⁻, CD10⁺, CD105⁺ placental cells, or isolated CD34⁻, CD10⁺, CD105⁺, CD200⁺ placental cells, are additionally CD44⁺. In a specific embodiment of any of the populations of cells comprising isolated CD34⁻, CD10⁺, CD105⁺ placental cells above, the isolated placental cells are additionally one or more of CD13⁺, CD29⁺, CD33⁺, CD38⁻, CD44⁺, CD45⁻, CD54⁺, CD62E⁻, CD62L⁻, CD62P⁻, SH3⁺ (CD73⁺), SH4⁺ (CD73⁺), CD80⁻, CD86⁻, CD90⁺, SH2⁺ (CD105⁺), CD106/VCAM⁺, CD117⁻, CD144/VE-cadherin^{low}, CD184/CXCR4⁻, CD200⁺, CD133⁻, OCT-4⁺, SSEA3⁻, SSEA4⁻, ABC-p⁺, KDR⁻ (VEGFR2⁻), HLA-A,B,C⁺. HLA-DP,DQ,DR⁻, HLA-G⁻, or Programmed Death-1 Ligand (PDL1)⁺, or any combination thereof. In another specific embodiment, the CD34⁻, CD10⁺, CD105⁺ cells are additionally CD13⁺, CD29⁺, CD33⁺, CD38⁻, CD44⁺, CD45⁻, CD54/ICAM⁺, CD62E⁻, CD62L⁻, CD62P⁻, SH3⁺ (CD73⁺), SH4⁺ (CD73⁺), CD80⁻, CD86⁻, CD90⁺, SH2⁺ (CD105⁺), CD106/VCAM⁺, CD117⁻, CD144/VE-cadherin^{low}, CD184/CXCR4⁻, CD200⁺, CD133⁻, OCT-4⁺, SSEA3⁻, SSEA4⁻, ABC-p⁺, KDR⁻ (VEGFR2⁻), HLA-A,B,C⁺, HLA-DP,DQ,DR⁻, HLA-G⁻, and Programmed Death-1 Ligand (PDL1)⁺.

In certain embodiments, the isolated placental cells useful in the methods and compositions described herein are one or more, or all, of CD10⁺, CD29⁺, CD34⁻, CD38⁻, CD44⁺, CD45⁻, CD54⁺, CD90⁺, SH2⁺, SH3⁺, SH4⁺, SSEA3⁻, SSEA4⁻, OCT-4⁺, and ABC-p⁺, wherein said isolated placental cells are obtained by physical and/or enzymatic disruption of placental tissue. In a specific embodiment, the isolated placental cells are OCT-4⁺ and ABC-p⁺. In another specific embodiment, the isolated placental cells are OCT-4⁺ and CD34⁻, wherein said isolated placental cells have at least one of the following characteristics: CD10⁺, CD29⁺, CD44⁺, CD45⁻, CD54⁺, CD90⁺, SH3⁺, SH4⁺, SSEA3⁻, and SSEA4⁻. In another specific embodiment, the isolated placental cells are OCT-4⁺, CD34⁻, CD10⁺, CD29⁺, CD44⁺, CD45⁻, CD54⁺, CD90⁺, SH3⁺, SH4⁺, SSEA3⁻, and SSEA4⁻. In another embodiment, the isolated placental cells are OCT-4⁺, CD34⁻, SSEA3⁻, and SSEA4⁻. In another specific embodiment, the isolated placental cells are OCT-4⁺ and CD34⁻, and is either SH2⁺ or SH3⁺. In another specific embodiment, the isolated placental cells are OCT-4⁺, CD34⁻, SH2⁺, and SH3⁺. In another specific embodiment, the isolated placental cells are OCT-4⁺, CD34⁻, SSEA3⁻, and SSEA4⁻, and are either SH2⁺ or SH3⁺. In another specific embodiment, the isolated placental cells are OCT-4⁺ and CD34⁻, and either SH2⁺ or SH3⁺, and is at least one of CD10⁺, CD29⁺, CD44⁺, CD45⁻, CD54⁺, CD90⁺, SSEA3⁻, or SSEA4⁻. In another specific embodiment, the isolated placental cells are OCT-4⁺, CD34⁻, CD10⁺, CD29⁺, CD44⁺, CD45⁻, CD54⁺, CD90⁺, SSEA3⁻, and SSEA4⁻, and either SH2⁺ or SH3⁺.

In another embodiment, the isolated placental cells useful in the methods and compositions disclosed herein are SH2⁺, SH3⁺, SH4⁺ and OCT-4⁺. In another specific embodiment, the isolated placental cells are CD10⁺, CD29⁺, CD44⁺, CD54⁺, CD90⁺, CD34⁻, CD45⁻, SSEA3⁻, or SSEA4⁻. In another embodiment, the isolated placental cells are SH2⁺, SH3⁺, SH4⁺. SSEA3⁻ and SSEA4⁻. In another specific embodiment, the isolated placental cells are SH2⁺, SH3⁺, SH4⁺, SSEA3⁻ and SSEA4⁻, CD10⁺, CD29⁺, CD44⁺, CD54⁺, CD90⁺, OCT-4⁺, CD34⁻ or CD45⁻.

In another embodiment, the isolated placental cells useful in the methods and compositions disclosed herein are CD10⁺, CD29⁺, CD34⁻, CD44⁺, CD45⁻, CD54⁺, CD90⁺, SH2⁺, SH3⁺, and SH4⁺; wherein said isolated placental cells are additionally one or more of OCT-4⁺, SSEA3⁻ or SSEA4⁻.

In certain embodiments, isolated placental cells useful in the methods and compositions disclosed herein are CD200⁺ or HLA-G⁻. In a specific embodiment, the isolated placental cells are CD200⁺ and HLA-G⁻. In another specific embodiment, the isolated placental cells are additionally CD73⁺ and CD105⁺. In another specific embodiment, the isolated placental cells are additionally CD34⁻, CD38⁻ or CD45⁻. In another specific embodiment, the isolated placental cells are additionally CD34⁻, CD38⁻ and CD45⁻. In another specific embodiment, said placental cells are CD34⁻, CD38⁻, CD45⁻, CD73⁺ and CD105⁺. In another specific embodiment, said isolated CD200⁺ or HLA-G⁻ placental cells facilitate the formation of embryoid-like bodies in a population of placental cells comprising the isolated placental cells, under conditions that allow the formation of embryoid-like bodies. In another specific embodiment, the isolated placental cells are isolated away from placental cells that are not stem or multipotent cells. In another specific embodiment, said isolated placental cells are isolated away from placental cells that do not display this combination of markers.

In another embodiment, a cell population useful in the methods and compositions described herein is a population of cells comprising, e.g., that is enriched for, CD200⁺, HLA-G⁻ stem cells. In a specific embodiment, said population is a population of placental cells. In various embodiments, at least about 10%, at least about 20%, at least about 30%, at least about 40%, at least about 50%, or at least about 60% of cells in said cell population are isolated CD200⁺, HLA-G⁻ placental cells. Preferably, at least about 70% of cells in said cell population are isolated CD200⁺, HLA-G⁻ placental cells. More preferably, at least about 90%, 95%, or 99% of said cells are isolated CD200⁺, HLA-G⁻ placental cells. In a specific embodiment of the cell populations, said isolated CD200⁺, HLA-G⁻ placental cells are also CD73⁺ and CD105⁺. In another specific embodiment, said isolated CD200⁺, HLA-G⁻ placental cells are also CD34⁻, CD38⁻ or CD45⁻. In another specific embodiment, said isolated CD200⁺, HLA-G⁻ placental cells are also CD34⁻, CD38⁻, CD45⁻, CD73⁺ and CD105⁺. In another embodiment, said cell population produces one or more embryoid-like bodies when cultured under conditions that allow the formation of embryoid-like bodies. In another specific embodiment, said cell population is isolated away from placental cells that are not stem cells. In another specific embodiment, said isolated CD200⁺, HLA-G⁻ placental cells are isolated away from placental cells that do not display these markers.

In another embodiment, the isolated placental cells useful in the methods and compositions described herein are CD73⁺, CD105⁺, and CD200⁺. In another specific embodiment, the isolated placental cells are HLA-G⁻. In another specific embodiment, the isolated placental cells are CD34⁻, CD38⁻ or CD45⁻. In another specific embodiment, the isolated placental cells are CD34⁻, CD38⁻ and CD45⁻. In another specific embodiment, the isolated placental cells are CD34⁻, CD38⁻, CD45⁻, and HLA-G⁻. In another specific embodiment, the isolated CD73⁺, CD105⁺, and CD200⁺ placental cells facilitate the formation of one or more embryoid-like bodies in a population of placental cells comprising the isolated placental cells, when the population is cultured under conditions that allow the formation of embryoid-like bodies. In another specific embodiment, the isolated placental cells are isolated away from placental cells that are not the isolated placental cells. In another specific embodiment, the isolated placental cells are isolated away from placental cells that do not display these markers.

In another embodiment, a cell population useful in the methods and compositions described herein is a population of cells comprising, e.g., that is enriched for, isolated CD73⁺, CD105⁺, CD200⁺ placental cells. In various embodiments, at least about 10%, at least about 20%, at least about 30%, at least about 40%, at least about 50%, or at least about 60% of cells in said cell population are isolated CD73⁺, CD105⁺, CD200⁺ placental cells. In another embodiment, at least about 70% of said cells in said population of cells are isolated CD73⁺, CD105⁺, CD200⁺ placental cells. In another embodiment, at least about 90%, 95% or 99% of cells in said population of cells are isolated CD73⁺, CD105⁺, CD200⁺ placental cells. In a specific embodiment of said populations, the isolated placental cells are HLA-G⁻. In another specific embodiment, the isolated placental cells are additionally CD34⁻, CD38⁻ or CD45⁻. In another specific embodiment, the isolated placental cells are additionally CD34⁻, CD38⁻ and CD45⁻. In another specific embodiment, the isolated placental cells are additionally CD34⁻, CD38⁻. CD45⁻, and HLA-G. In another specific embodiment, said population of cells produces one or more embryoid-like bodies when cultured under conditions that allow the formation of embryoid-like bodies. In another specific embodiment, said population of placental cells is isolated away from placental cells that are not stem cells. In another specific embodiment, said population of placental cells is isolated away from placental cells that do not display these characteristics.

In certain other embodiments, the isolated placental cells are one or more of CD10⁺, CD29⁺, CD34⁻, CD38⁻, CD44⁺, CD45⁻, CD54⁺, CD90⁺, SH2⁻, SH4⁺, SSEA3−, SSEA4⁻, OCT-4⁺, HLA-G⁻ or ABC-p⁺. In a specific embodiment, the isolated placental cells are CD10⁺, CD29⁺. CD34⁻, CD38⁻, CD44⁺, CD45⁻, CD54⁺, CD90⁺, SH2⁺, SH3⁺, SH4⁺, SSEA3−, SSEA4⁻, and OCT-4⁺. In another specific embodiment, the isolated placental cells are CD10⁺, CD29⁺, CD34⁻, CD38⁻, CD45⁻, CD54⁺, SH2⁺, SH3⁺, and SH4⁺. In another specific embodiment, the isolated placental cells are CD10⁺, CD29⁺, CD34⁻, CD38⁻, CD45⁻, CD54⁺, SH2⁺, SH3⁺, SH4⁺ and OCT-4⁺. In another specific embodiment, the isolated placental cells are CD10⁺, CD29⁺, CD34⁻, CD38⁻, CD44⁺, CD45⁻, CD54⁺, CD90⁺, HLA-G⁻, SH2⁺, SH3⁺, SH4⁺. In another specific embodiment, the isolated placental cells are OCT-4⁺ and ABC-p⁺. In another specific embodiment, the isolated placental cells are SH2⁺, SH3⁺, SH4⁺ and OCT-4⁺. In another embodiment, the isolated placental cells are OCT-4⁺, CD34⁻, SSEA3⁻, and SSEA4⁻. In a specific embodiment, said isolated OCT-4⁺, CD34⁻, SSEA3⁻, and SSEA4⁻ placental cells are additionally CD10⁺, CD29⁺, CD34⁻, CD44⁺, CD45⁻, CD54⁺, CD90⁺, SH2⁺, SH3⁺, and SH4⁺. In another embodiment, the isolated placental cells are OCT-4⁺ and CD34⁻, and either SH3⁺ or SH4⁺. In another embodiment, the isolated placental cells are CD34⁻ and either CD10⁺, CD29⁺, CD44⁺, CD54⁺, CD90⁺, or OCT-4⁺.

In another embodiment, the isolated placental cells useful in the methods and compositions described herein are CD200⁺ and OCT-4⁺. In a specific embodiment, the isolated placental cells are CD73⁺ and CD105⁺. In another specific embodiment, said isolated placental cells are HLA-G⁻. In another specific embodiment, said isolated CD200⁺, OCT-4⁺ placental cells are CD34⁻, CD38⁻ or CD45⁻. In another specific embodiment, said isolated CD200⁺, OCT-4⁺ placental cells are CD34⁻, CD38⁻ and CD45⁻. In another specific embodiment, said isolated CD200⁺, OCT-4⁺ placental cells are CD34⁻, CD38⁻, CD45⁻, CD73⁺, CD105⁺ and HLA-G⁻. In another specific embodiment, the isolated CD200⁺, OCT-4⁺ placental cells facilitate the production of one or more embryoid-like bodies by a population of placental cells that comprises the isolated cells, when the population is cultured under conditions that allow the formation of embryoid-like bodies. In another specific embodiment, said isolated CD200⁺, OCT-4⁺ placental cells are isolated away from placental cells that are not stem cells. In another specific embodiment, said isolated CD200⁺, OCT-4⁺ placental cells are isolated away from placental cells that do not display these characteristics.

In another embodiment, a cell population useful in the methods and compositions described herein is a population of cells comprising, e.g., that is enriched for, CD200⁺, OCT-4⁺ placental cells. In various embodiments, at least about 10%, at least about 20%, at least about 30%, at least about 40%, at least about 50%, or at least about 60% of cells in said cell population are isolated CD200⁺, OCT-4⁺ placental cells. In another embodiment, at least about 70% of said cells are said isolated CD200⁺, OCT-4⁺ placental cells. In another embodiment, at least about 80%, 90%, 95%, or 99% of cells in said cell population are said isolated CD200⁺, OCT-4⁺ placental cells. In a specific embodiment of the isolated populations, said isolated CD200⁺, OCT-4⁺ placental cells are additionally CD73⁺ and CD105⁺. In another specific embodiment, said isolated CD200⁺, OCT-4⁺ placental cells are additionally HLA-G⁻. In another specific embodiment, said isolated CD200⁺, OCT-4⁺ placental cells are additionally CD34⁻, CD38⁻ and CD45⁻. In another specific embodiment, said isolated CD200⁺, OCT-4⁺ placental cells are additionally CD34⁻, CD38⁻, CD45⁻, CD73⁺, CD105⁺ and HLA-G⁻. In another specific embodiment, the cell population produces one or more embryoid-like bodies when cultured under conditions that allow the formation of embryoid-like bodies. In another specific embodiment, said cell population is isolated away from placental cells that are not isolated CD200⁺, OCT-4⁺ placental cells. In another specific embodiment, said cell population is isolated away from placental cells that do not display these markers.

In another embodiment, the isolated placental cells useful in the methods and compositions described herein are CD73⁺, CD105⁺ and HLA-G⁻. In another specific embodiment, the isolated CD73⁺, CD105⁺ and HLA-G⁻ placental cells are additionally CD34⁻, CD38⁻ or CD45⁻. In another specific embodiment, the isolated CD73⁺, CD105⁺, HLA-G⁻ placental cells are additionally CD34⁻, CD38⁻ and CD45⁻. In another specific embodiment, the isolated CD73⁺, CD105⁺, HLA-G⁻ placental cells are additionally OCT-4⁺.

In another specific embodiment, the isolated CD73⁺, CD105⁺, HLA-G⁻ placental cells are additionally CD200⁺. In another specific embodiment, the isolated CD73⁺, CD105⁺, HLA-G⁻ placental cells are additionally CD34⁻, CD38⁻, CD45⁻, OCT-4⁺ and CD200⁺. In another specific embodiment, the isolated CD73⁺, CD105⁺, HLA-G⁻ placental cells facilitate the formation of embryoid-like bodies in a population of placental cells comprising said cells, when the population is cultured under conditions that allow the formation of embryoid-like bodies. In another specific embodiment, said the isolated CD73⁺, CD105⁺, HLA-G⁻ placental cells are isolated away from placental cells that are not the isolated CD73⁺, CD105⁺, HLA-G⁻ placental cells. In another specific embodiment, said the isolated CD73⁺, CD105⁺, HLA-G⁻ placental cells are isolated away from placental cells that do not display these markers.

In another embodiment, a cell population useful in the methods and compositions described herein is a population of cells comprising, e.g., that is enriched for, isolated CD73⁺, CD105 and HLA-G⁻ placental cells. In various embodiments, at least about 10%, at least about 20%, at least about 30%, at least about 40%, at least about 50%, or at least about 60% of cells in said population of cells are isolated CD73⁺, CD105⁺, HLA-G⁻placental cells. In another embodiment, at least about 70% of cells in said population of cells are isolated CD73⁺, CD105⁺, HLA-G⁻ placental cells. In another embodiment, at least about 90%, 95% or 99% of cells in said population of cells are isolated CD73⁺, CD105⁺, HLA-G⁻ placental cells. In a specific embodiment of the above populations, said isolated CD73⁺, CD105⁺, HLA-G⁻ placental cells are additionally CD34⁻, CD38⁻ or CD45⁻. In another specific embodiment, said isolated CD73⁺, CD105⁺, HLA-G⁻ placental cells are additionally CD34⁻, CD38⁻ and CD45⁻. In another specific embodiment, said isolated CD73⁺, CD105⁺, HLA-G⁻ placental cells are additionally OCT-4⁺. In another specific embodiment, said isolated CD73⁺, CD105⁺, HLA-G⁻ placental cells are additionally CD200⁺. In another specific embodiment, said isolated CD73⁺, CD105⁺, HLA-G⁻ placental cells are additionally CD34⁻, CD38⁻, CD45⁻, OCT-4⁺ and CD200⁺. In another specific embodiment, said cell population is isolated away from placental cells that are not CD73⁺, CD105⁺, HLA-G⁻ placental cells. In another specific embodiment, said cell population is isolated away from placental cells that do not display these markers.

In another embodiment, the isolated placental cells useful in the methods and compositions described herein are CD73⁺ and CD105⁺ and facilitate the formation of one or more embryoid-like bodies in a population of isolated placental cells comprising said CD73⁺, CD105⁺ cells when said population is cultured under conditions that allow formation of embryoid-like bodies. In another specific embodiment, said isolated CD73⁺, CD105⁺ placental cells are additionally CD34⁻, CD38⁻ or CD45⁻. In another specific embodiment, said isolated CD73⁺, CD105⁺ placental cells are additionally CD34⁻, CD38⁻ and CD45⁻. In another specific embodiment, said isolated CD73⁺, CD105⁺ placental cells are additionally OCT-4⁺. In another specific embodiment, said isolated CD73⁺, CD105⁺ placental cells are additionally OCT-4⁺, CD34⁻, CD38⁻ and CD45⁻. In another specific embodiment, said isolated CD73⁺, CD105⁺ placental cells are isolated away from placental cells that are not said cells. In another specific embodiment, said isolated CD73⁺, CD105⁺ placental cells are isolated away from placental cells that do not display these characteristics.

In another embodiment, a cell population useful in the methods and compositions described herein is a population of cells comprising, e.g., that is enriched for, isolated placental cells that are CD73⁺, CD105⁺ and facilitate the formation of one or more embryoid-like bodies in a population of isolated placental cells comprising said cells when said population is cultured under conditions that allow formation of embryoid-like bodies. In various embodiments, at least about 10%, at least about 20%, at least about 30%, at least about 40%, at least about 50%, or at least about 60% of cells in said population of cells are said isolated CD73⁺, CD105⁺ placental cells. In another embodiment, at least about 70% of cells in said population of cells are said isolated CD73⁺, CD105⁺ placental cells. In another embodiment, at least about 90%, 95% or 99% of cells in said population of cells are said isolated CD73⁺, CD105⁺ placental cells. In a specific embodiment of the above populations, said isolated CD73⁺, CD105⁺ placental cells are additionally CD34⁻, CD38⁻ or CD45⁻. In another specific embodiment, said isolated CD73⁺, CD105⁺ placental cells are additionally CD34⁻, CD38⁻ and CD45⁻. In another specific embodiment, said isolated CD73⁺, CD105⁺ placental cells are additionally OCT-4⁺. In another specific embodiment, said isolated CD73⁺, CD105⁺ placental cells are additionally CD200⁺. In another specific embodiment, said isolated CD73⁺, CD105⁺ placental cells are additionally CD34⁻, CD38⁻, CD45⁻, OCT-4⁺ and CD200⁺. In another specific embodiment, said cell population is isolated away from placental cells that are not said isolated CD73⁺, CD105⁺ placental cells. In another specific embodiment, said cell population is isolated away from placental cells that do not display these markers.

In another embodiment, the isolated placental cells useful in the methods and compositions described herein are OCT-4⁺ and facilitate formation of one or more embryoid-like bodies in a population of isolated placental cells comprising said cells when cultured under conditions that allow formation of embryoid-like bodies. In a specific embodiment, said isolated OCT-4⁺ placental cells are additionally CD73⁺ and CD105⁺. In another specific embodiment, said isolated OCT-4⁺ placental cells are additionally CD34⁻, CD38⁻, or CD45⁻. In another specific embodiment, said isolated OCT-4⁺ placental cells are additionally CD200⁺. In another specific embodiment, said isolated OCT-4⁺ placental cells are additionally CD73⁺, CD105⁺, CD200⁺, CD34⁻, CD38⁻, and CD45⁻. In another specific embodiment, said isolated OCT-4⁺ placental cells are isolated away from placental cells that are not OCT-4⁺ placental cells. In another specific embodiment, said isolated OCT-4⁺ placental cells are isolated away from placental cells that do not display these characteristics.

In another embodiment, a cell population useful in the methods and compositions described herein is a population of cells comprising, e.g., that is enriched for, isolated placental cells that are OCT-4″ and facilitate the formation of one or more embryoid-like bodies in a population of isolated placental cells comprising said cells when said population is cultured under conditions that allow formation of embryoid-like bodies. In various embodiments, at least about 10%, at least about 20%, at least about 30%, at least about 40%, at least about 50%, or at least about 60% of cells in said population of cells are said isolated OCT-4⁺ placental cells. In another embodiment, at least about 70% of cells in said population of cells are said isolated OCT-4⁺ placental cells. In another embodiment, at least about 80%, 90%, 95% or 99% of cells in said population of cells are said isolated OCT-4⁺ placental cells. In a specific embodiment of the above populations, said isolated OCT-4⁺ placental cells are additionally CD34⁻, CD38⁺ or CD45⁻. In another specific embodiment, said isolated OCT-4⁺ placental cells are additionally CD34⁻, CD38⁻ and CD45⁻. In another specific embodiment, said isolated OCT-4⁺ placental cells are additionally CD73⁺ and CD105⁺. In another specific embodiment, said isolated OCT-4⁺ placental cells are additionally CD200⁺. In another specific embodiment, said isolated OCT-4⁺ placental cells are additionally CD73⁺, CD105⁺, CD200⁺, CD34⁻, CD38⁻, and CD45⁻. In another specific embodiment, said cell population is isolated away from placental cells that are not said cells. In another specific embodiment, said cell population is isolated away from placental cells that do not display these markers.

In another embodiment, the isolated placental cells useful in the methods and compositions described herein are isolated HLA-A,B,C⁺, CD45⁻, CD133⁻ and CD34⁻ placental cells. In another embodiment, a cell population useful in the methods and compositions described herein is a population of cells comprising isolated placental cells, wherein at least about 70%, at least about 80%, at least about 90%, at least about 95% or at least about 99% of cells in said population of cells are isolated HLA-A,B,C⁺, CD45⁻, CD133⁻ and CD34⁻ placental cells. In a specific embodiment, said isolated placental cell or population of isolated placental cells is isolated away from placental cells that are not HLA-A,B,C⁺, CD45⁻, CD133⁻ and CD34⁻ placental cells. In another specific embodiment, said isolated placental cells are non-maternal in origin. In another specific embodiment, said population of isolated placental cells are substantially free of maternal components; e.g., at least about 40%, 45%, 5-0%, 55%, 60%, 65%, 70%, 75%, 90%, 85%, 90%, 95%, 98% or 99% of said cells in said population of isolated placental cells are non-maternal in origin.

In another embodiment, the isolated placental cells useful in the methods and compositions described herein are isolated CD10⁺, CD13⁺, CD33⁺, CD45⁻, CD117⁻ and CD133⁻ placental cells. In another embodiment, a cell population useful in the methods and compositions described herein is a population of cells comprising isolated placental cells, wherein at least about 70%, at least about 80%, at least about 90%, at least about 95% or at least about 99% of cells in said population of cells are isolated CD10⁺, CD13⁺, CD33⁺, CD45⁻, CD117⁻ and CD133⁻ placental cells. In a specific embodiment, said isolated placental cells or population of isolated placental cells is isolated away from placental cells that are not said isolated placental cells. In another specific embodiment, said isolated CD10⁺, CD13⁺, CD33⁺, CD45⁻, CD117⁻ and CD133⁻ placental cells are non-maternal in origin, i.e., have the fetal genotype. In another specific embodiment, at least about 40%, 45%, 50%, 55%, 60%, 65%, 70%, 75%, 90%, 85%, 90%, 95%, 98% or 99% of said cells in said population of isolated placental cells, are non-maternal in origin. In another specific embodiment, said isolated placental cells or population of isolated placental cells are isolated away from placental cells that do not display these characteristics.

In another embodiment, the isolated placental cells useful in the methods and compositions described herein are isolated CD10⁺ CD33⁻, CD44⁺, CD45⁻, and CD117⁻ placental cells. In another embodiment, a cell population useful for the in the methods and compositions described herein is a population of cells comprising, e.g., enriched for, isolated placental cells, wherein at least about 70%, at least about 80%, at least about 90%, at least about 95% or at least about 99% of cells in said population of cells are isolated CD10⁺ CD33⁻, CD44⁺, CD45⁻, and CD117⁻ placental cells. In a specific embodiment, said isolated placental cell or population of isolated placental cells is isolated away from placental cells that are not said cells. In another specific embodiment, said isolated placental cells are non-maternal in origin. In another specific embodiment, at least about 40%, 45%, 50%, 55%, 60%, 65%, 70%, 75%, 90%, 85%, 90%, 95%, 98% or 99% of said cells in said cell population are non-maternal in origin. In another specific embodiment, said isolated placental cell or population of isolated placental cells is isolated away from placental cells that do not display these markers.

In another embodiment, the isolated placental cells useful in the methods and compositions described herein are isolated CD10⁺ CD13⁻, CD33⁻, CD45⁻, and CD117⁻ placental cells. In another embodiment, a cell population useful in the methods and compositions described herein is a population of cells comprising, e.g., enriched for, isolated CD10⁺, CD13⁻, CD33⁻, CD45⁻, and CD117⁻ placental cells, wherein at least about 70%, at least about 80%, at least about 90%, at least about 95% or at least about 99% of cells in said population are CD10+ CD13⁻, CD33⁻, CD45⁻, and CD117⁻ placental cells. In a specific embodiment, said isolated placental cells or population of isolated placental cells are isolated away from placental cells that are not said cells. In another specific embodiment, said isolated placental cells are non-maternal in origin. In another specific embodiment, at least about 40%, 45%, 50%, 55%, 60%, 65%, 70%, 75%, 90%, 85%, 90%, 95%, 98% or 99% of said cells in said cell population are non-maternal in origin. In another specific embodiment, said isolated placental cells or population of isolated placental cells is isolated away from placental cells that do not display these characteristics.

In another embodiment, the isolated placental cells useful in the methods and compositions described herein are HLA A,B,C⁺, CD45⁻, CD34⁻, and CD133⁻, and are additionally CD10⁺, CD13⁺, CD38⁺, CD44⁺, CD90⁺, CD105⁺, CD200⁺ and/or HLA-G⁻, and/or negative for CD117. In another embodiment, a cell population useful in the methods described herein is a population of cells comprising isolated placental cells, wherein at least about 20%, 25%, 30%, 35%, 40%, 45%, 50%, 55%, 60%, 65%, 70%, 75%, 80%, 85%, 90%, 95%, 98% or about 99% of the cells in said population are isolated placental cells that are HLA A,B,C⁻, CD45⁻, CD34⁻, CD133⁻, and that are additionally positive for CD10, CD13, CD38, CD44, CD90, CD105, CD200, and/or negative for CD117 and/or HLA-G. In a specific embodiment, said isolated placental cells or population of isolated placental cells are isolated away from placental cells that are not said cells. In another specific embodiment, said isolated placental cells are non-maternal in origin. In another specific embodiment, at least about 40%, 45%, 50%, 55%, 60%, 65%, 70%, 75%, 90%, 85%, 90%, 95%, 98% or 99% of said cells in said cell population are non-maternal in origin. In another specific embodiment, said isolated placental cells or population of isolated placental cells are isolated away from placental cells that do not display these markers.

In another embodiment, the isolated placental cells useful in the methods and compositions described herein are isolated placental cells that are CD200⁺ and CD10⁺, as determined by antibody binding, and CD117⁻, as determined by both antibody binding and RT-PCR. In another embodiment, the isolated placental cells useful in the methods and compositions described herein are isolated placental cells, e.g., placental stem cells or placental multipotent cells, that are CD10⁺, CD29⁻, CD54⁺, CD200⁺, HLA-G⁻, MHC class I⁺ and β-2-microglobulin⁺. In another embodiment, isolated placental cells useful in the methods and compositions described herein are placental cells wherein the expression of at least one cellular marker is at least two-fold higher than for a mesenchymal stem cell (e.g., a bone marrow-derived mesenchymal stem cell). In another specific embodiment, said isolated placental cells are non-maternal in origin. In another specific embodiment, at least about 40%, 45%, 50%, 55%, 60%, 65%, 70%, 75%, 90%, 85%, 90%, 95%, 98% or 99% of said cells in said cell population are non-maternal in origin.

In another embodiment, the isolated placental cells useful in the methods and compositions described herein are isolated placental cells, e.g., placental stem cells or placental multipotent cells, that are one or more of CD10⁺, CD29⁺, CD44⁺, CD45⁻, CD54/ICAM⁺, CD62E⁻. CD62L⁻, CD62P⁻, CD80⁻, CD86⁻, CD103⁻, CD104⁻, CD105⁺, CD106/VCAM⁺, CD144/VE-cadherin^{low}, CD184/CXCR4⁻, β-microglobulin^{low}, MHC-I^{low}, MHC-II⁻, HLA-G^{low}, and/or PDL1^{low}. In a specific embodiment, the isolated placental cells are at least CD29⁺ and CD54⁺. In another specific embodiment, the isolated placental cells are at least CD44⁺ and CD106⁺. In another specific embodiment, the isolated placental cells are at least CD29⁺.

In another embodiment, a cell population useful in the methods and compositions described herein comprises isolated placental cells, and at least 50%, 60%, 70%, 80%, 90%, 95%, 98% or 99% of the cells in said cell population are isolated placental cells that are one or more of CD10⁺, CD29⁺, CD44⁺, CD45⁻, CD54/ICAM⁺, CD62-E⁻, CD62-L⁻, CD62-P⁻, CD80⁻, CD86⁻, CD103⁻, CD104⁻, CD105⁺, CD106/VCAM⁺, CD144/VE-cadherin^{dim}, CD184/CXCR4⁻, β-microglobulin^{dim}, HLA-I^{dim}, HLA-II⁻, HLA-G^{dim}, and/or PDL1^{dim}. In another specific embodiment, at least 50%, 60%, 70%, 80%, 90%, 95%, 98% or 99% of cells in said cell population are CD10⁺, CD29⁺, CD44⁺, CD45⁻, CD54/ICAM⁺, CD62-E⁻, CD62-L⁻, CD62-P⁻, CD80⁻, CD86⁻, CD103⁻, CD104⁻, CD105⁺, CD106/VCAM⁺, CD144/VE-cadherin^{dim}, CD184/CXCR4⁻, β-microglobulin^{dim}, MHC-I^{dim}, MHC-II⁻, HLA-G^{dim}, and PDL1^{dim}. In certain embodiments, the placental cells express HLA-II markers when induced by interferon gamma (IFN-γ).

In another embodiment, the isolated placental cells useful in the methods and compositions described herein are isolated placental cells that are one or more, or all, of CD10⁺, CD29⁺, CD34⁻, CD38⁻, CD44⁺, CD45⁻, CD54⁺, CD90⁺, SH2⁺, SH3⁺, SH4⁺, SSEA3⁻, SSEA4⁻, OCT-4⁺, and ABC-p⁺, where ABC-p is a placenta-specific ABC transporter protein (also known as breast cancer resistance protein (BCRP) and as mitoxantrone resistance protein (MXR)), wherein said isolated placental cells are obtained by perfusion of a mammalian, e.g., human, placenta that has been drained of cord blood and perfused to remove residual blood.

In another specific embodiment of any of the above characteristics, expression of the cellular marker (e.g., cluster of differentiation or immunogenic marker) is determined by flow cytometry; in another specific embodiment, expression of the marker is determined by RT-PCR.

Gene profiling confirms that isolated placental cells, and populations of isolated placental cells, are distinguishable from other cells, e.g., mesenchymal stem cells, e.g., bone marrow-derived mesenchymal stem cells. The isolated placental cells described herein can be distinguished from, e.g., mesenchymal stem cells on the basis of the expression of one or more genes, the expression of which is significantly higher in the isolated placental cells in comparison to bone marrow-derived mesenchymal stem cells. In particular, the isolated placental cells, useful in the methods of treatment provided herein, can be distinguished from mesenchymal stem cells on the basis of the expression of one or more genes, the expression of which is significantly higher (that is, at least twofold higher) in the isolated placental cells than in an equivalent number of bone marrow-derived mesenchymal stem cells, wherein the one or more genes are ACTG2, ADARB1, AMIGO2, ARTS-1, B4GALT6, BCHE, Cl lorf9, CD200, COL4A1, COL4A2, CPA4, DMD, DSC3, DSG2, ELOVL2, F2RL1, FLJ10781, GATA6, GPR126, GPRC5B, ICAM1, IER3, IGFBP7, ILIA, IL6, IL18, KRT18, KRT8, LIPG, LRAP, MATN2, MEST, NFE2L3, NUAK1, PCDH7, PDLIM3, PKP2, RTN1, SERPINB9, ST3GAL6, ST6GALNAC5, SLC12A8, TCF21, TGFB2, VTN, ZC3H12A, or a combination of any of the foregoing, when the cells are grown under equivalent conditions. See, e.g., U.S. Patent Application Publication No. 2007/0275362, the disclosure of which is incorporated herein by reference in its entirety. In certain specific embodiments, said expression of said one or more genes is determined, e.g., by RT-PCR or microarray analysis, e.g, using a U133-A microarray (Affymetrix). In another specific embodiment, said isolated placental cells express said one or more genes when cultured for a number of population doublings, e.g., anywhere from about 3 to about 35 population doublings, in a medium comprising DMEM-LG (e.g., from Gibco); 2% fetal calf serum (e.g., from Hyclone Labs.); 1× insulin-transferrin-selenium (ITS); 1× linoleic acid-bovine serum albumin (LA-BSA); 10⁻⁹ M dexamethasone (e.g., from Sigma); 10⁻⁴ M ascorbic acid 2-phosphate (e.g., from Sigma); epidermal growth factor 10 ng/mL (e.g., from R&D Systems); and platelet-derived growth factor (PDGF-BB) 10 ng/mL (e.g., from R&D Systems). In another specific embodiment, the isolated placental cell-specific gene is CD200.

Specific sequences for these genes can be found in GenBank at accession nos. NM_{—}001615 (ACTG2), BC065545 (ADARB1), (NM_{—}181847 (AMIGO2), AY358590 (ARTS-1), BC074884 (B4GALT6), BC008396 (BCHE), BCO20196 (Cl lorf9), BCO31103 (CD200), NM_{—}001845 (COL4A1), NM_{—}001846 (COL4A2), BCO52289 (CPA4), BC094758 (DMD), AF293359 (DSC3), NM_{—}001943 (DSG2), AF338241 (ELOVL2), AY336105 (F2RL1), NM_{—}018215 (FLJ10781), AY416799 (GATA6), BC075798 (GPR126), NM_{—}016235 (GPRC5B), AF340038 (ICAM1), BC000844 (IER3), BC066339 (IGFBP7), BC013142 (IL1A), BT019749 (IL6), BC007461 (IL18), (BC072017) KRT18, BC075839 (KRT8), BC060825 (LIPG), BC065240 (LRAP), BC010444 (MATN2), BC011908 (MEST), BC068455 (NFE2L3), NM_{—}014840 (NUAK1), AB006755 (PCDH7), NM_{—}014476 (PDLIM3), BC126199 (PKP-2), BC090862 (RTN1), BC002538 (SERPINB9), BCO23312 (ST3GAL6), BC001201 (ST6GALNAC5), BC126160 or BC065328 (SLC12A8), BCO25697 (TCF21), BC096235 (TGFB2), BC005046 (VTN), and BC005001 (ZC3H12A) as of March 2008.

In certain specific embodiments, said isolated placental cells express each of ACTG2, ADARB1, AMIGO2, ARTS-1, B4GALT6, BCHE, C11orf9, CD200, COL4A1, COL4A2, CPA4, DMD, DSC3, DSG2, ELOVL2, F2RL1, FLJ10781, GATA6, GPR126, GPRC5B, ICAM1, IER3, IGFBP7, ILIA, IL6, IL18, KRT18, KRT8, LIPG, LRAP, MATN2, MEST, NFE2L3, NUAK1, PCDH7, PDLIM3, PKP2, RTN1, SERPINB9, ST3GAL6, ST6GALNAC5, SLC12A8, TCF21, TGFB2, VTN, and ZC3H12A at a detectably higher level than an equivalent number of bone marrow-derived mesenchymal stem cells, when the cells are grown under equivalent conditions.

In specific embodiments, the placental cells express CD200 and ARTS1 (aminopeptidase regulator of type 1 tumor necrosis factor); ARTS-1 and LRAP (leukocyte-derived arginine aminopeptidase); IL6 (interleukin-6) and TGFB2 (transforming growth factor, beta 2); IL6 and KRT18 (keratin 18); IER3 (immediate early response 3), MEST (mesoderm specific transcript homolog) and TGFB2; CD200 and IER3; CD200 and IL6; CD200 and KRT18; CD200 and LRAP; CD200 and MEST; CD200 and NFE2L3 (nuclear factor (erythroid-derived 2)-like 3); or CD200 and TGFB2 at a detectably higher level than an equivalent number of bone marrow-derived mesenchymal stem cells (BM-MSCs) wherein said bone marrow-derived mesenchymal stem cells have undergone a number of passages in culture equivalent to the number of passages said isolated placental cells have undergone. In other specific embodiments, the placental cells express ARTS-1, CD200, IL6 and LRAP; ARTS-1, IL6, TGFB2, IER3, KRT18 and MEST; CD200, IER3, IL6, KRT18, LRAP, MEST, NFE2L3, and TGFB2; ARTS-1, CD200, IER3, IL6, KRT18, LRAP, MEST, NFE2L3, and TGFB2; or IER3, MEST and TGFB2 at a detectably higher level than an equivalent number of bone marrow-derived mesenchymal stem cells BM-MSCs, wherein said bone marrow-derived mesenchymal stem cells have undergone a number of passages in culture equivalent to the number of passages said isolated placental cells have undergone.

Expression of the above-referenced genes can be assessed by standard techniques. For example, probes based on the sequence of the gene(s) can be individually selected and constructed by conventional techniques. Expression of the genes can be assessed, e.g., on a microarray comprising probes to one or more of the genes, e.g. an Affymetrix GENECHIP® Human Genome U133A 2.0 array, or an Affymetrix GENECHIP® Human Genome U133 Plus 2.0 (Santa Clara, Calif.). Expression of these genes can be assessed even if the sequence for a particular GenBank accession number is amended because probes specific for the amended sequence can readily be generated using well-known standard techniques.

The level of expression of these genes can be used to confirm the identity of a population of isolated placental cells, to identify a population of cells as comprising at least a plurality of isolated placental cells, or the like. Populations of isolated placental cells, the identity of which is confirmed, can be clonal, e.g., populations of isolated placental cells expanded from a single isolated placental cell, or a mixed population of stem cells, e.g., a population of cells comprising solely isolated placental cells that are expanded from multiple isolated placental cells, or a population of cells comprising isolated placental cells, as described herein, and at least one other type of cell.

The level of expression of these genes can be used to select populations of isolated placental cells. For example, a population of cells, e.g., clonally-expanded cells, may be selected if the expression of one or more of the genes listed above is significantly higher in a sample from the population of cells than in an equivalent population of mesenchymal stem cells. Such selecting can be of a population from a plurality of isolated placental cell populations, from a plurality of cell populations, the identity of which is not known, etc.

Isolated placental cells can be selected on the basis of the level of expression of one or more such genes as compared to the level of expression in said one or more genes in, e.g., a mesenchymal stem cell control, for example, the level of expression in said one or more genes in an equivalent number of bone marrow-derived mesenchymal stem cells. In one embodiment, the level of expression of said one or more genes in a sample comprising an equivalent number of mesenchymal stem cells is used as a control. In another embodiment, the control, for isolated placental cells tested under certain conditions, is a numeric value representing the level of expression of said one or more genes in mesenchymal stem cells under said conditions.

The isolated placental cells described herein display the above characteristics (e.g., combinations of cell surface markers and/or gene expression profiles) in primary culture, or during proliferation in medium comprising, e.g., DMEM-LG (Gibco), 2% fetal calf serum (FCS) (Hyclone Laboratories), 1× insulin-transferrin-selenium (ITS), 1× linoleic-acid-bovine-serum-albumin (LA-BSA), 10⁻⁹M dexamethasone (Sigma), 10⁻⁴M ascorbic acid 2-phosphate (Sigma), epidermal growth factor (EGF)10 ng/ml (R&D Systems), platelet derived-growth factor (PDGF-BB) 10 ng/ml (R&D Systems), and 100 U penicillin/1000U streptomycin.

In certain embodiments of any of the placental cells disclosed herein, the cells are human. In certain embodiments of any of the placental cells disclosed herein, the cellular marker characteristics or gene expression characteristics are human markers or human genes.

In another specific embodiment of said isolated placental cells or populations of cells comprising the isolated placental cells, said cells or population have been expanded, for example, passaged at least, about, or no more than, 1, 2, 3, 4, 5, 6, 7, 8, 9, 10, 11, 12, 13, 14, 15, 16, 17, 18, 19, or 20 times, or proliferated for at least, about, or no more than, 1, 2, 3, 4, 5, 6, 7, 8, 9, 10, 12, 14, 16, 18, 20, 22, 24, 26, 28, 30, 32, 34, 36, 38 or 40 population doublings. In another specific embodiment of said isolated placental cells or populations of cells comprising the isolated placental cells, said cells or population are primary isolates. In another specific embodiment of the isolated placental cells, or populations of cells comprising isolated placental cells, that are disclosed herein, said isolated placental cells are fetal in origin (that is, have the fetal genotype).

In certain embodiments, said isolated placental cells do not differentiate during culturing in growth medium, i.e., medium formulated to promote proliferation, e.g., during proliferation in growth medium. In another specific embodiment, said isolated placental cells do not require a feeder layer in order to proliferate. In another specific embodiment, said isolated placental cells do not differentiate in culture in the absence of a feeder layer, solely because of the lack of a feeder cell layer.

In another embodiment, cells useful in the methods and compositions described herein are isolated placental cells, wherein a plurality of said isolated placental cells are positive for aldehyde dehydrogenase (ALDH), as assessed by an aldehyde dehydrogenase activity assay. Such assays are known in the art (see, e.g., Bostian and Betts, Biochem. J., 173, 787, (1978)). In a specific embodiment, said ALDH assay uses ALDEFLUOR® (Aldagen, Inc., Ashland, Oreg.) as a marker of aldehyde dehydrogenase activity. In a specific embodiment, said plurality is between about 3% and about 25% of cells in said population of cells. In another embodiment, said population of isolated placental cells shows at least three-fold, or at least five-fold, higher ALDH activity than a population of bone marrow-derived mesenchymal stem cells having about the same number of cells and cultured under the same conditions.

In certain embodiments of any of the populations of cells comprising the isolated placental cells described herein, the placental cells in said populations of cells are substantially free of cells having a maternal genotype; e.g., at least 40%, 45%, 50%, 55%, 60%, 65%, 70%, 75%, 80%, 85%, 90%, 95%, 98% or 99% of the placental cells in said population have a fetal genotype. In certain other embodiments of any of the populations of cells comprising the isolated placental cells described herein, the populations of cells comprising said placental cells are substantially free of cells having a maternal genotype; e.g., at least 40%, 45%, 50%, 55%, 60%, 65%, 70%, 75%, 80%, 85%, 90%, 95%, 98% or 99% of the cells in said population have a fetal genotype.

In a specific embodiment of any of the above isolated placental cells or cell populations of isolated placental cells, the karyotype of the cells, e.g., all of the cells, or at least about 95% or about 99% of the cells in said population, is normal. In another specific embodiment of any of the above placental cells or cell populations, the cells, or cells in the population of cells, are non-maternal in origin.

In a specific embodiment of any of the embodiments of placental cells disclosed herein, the placental cells are genetically stable, displaying a normal diploid chromosome count and a normal karyotype.

Isolated placental cells, or populations of isolated placental cells, bearing any of the above combinations of markers, can be combined in any ratio. Any two or more of the above isolated placental cell populations can be combined to form an isolated placental cell population. For example, a population of isolated placental cells can comprise a first population of isolated placental cells defined by one of the marker combinations described above, and a second population of isolated placental cells defined by another of the marker combinations described above, wherein said first and second populations are combined in a ratio of about 1:99, 2:98, 3:97, 4:96, 5:95, 10:90, 20:80, 30:70, 40:60, 50:50, 60:40, 70:30, 80:20, 90:10, 95:5, 96:4, 97:3, 98:2, or about 99:1. In like fashion, any three, four, five or more of the above-described isolated placental cells or isolated placental cells populations can be combined.

Isolated placental cells useful in the methods and compositions described herein can be obtained, e.g., by disruption of placental tissue, with or without enzymatic digestion (see Section 5.3.3) or perfusion (see Section 5.3.4). For example, populations of isolated placental cells can be produced according to a method comprising perfusing a mammalian placenta that has been drained of cord blood and perfused to remove residual blood; perfusing said placenta with a perfusion solution; and collecting said perfusion solution, wherein said perfusion solution after perfusion comprises a population of placental cells that comprises isolated placental cells; and isolating a plurality of said isolated placental cells from said population of cells. In a specific embodiment, the perfusion solution is passed through both the umbilical vein and umbilical arteries and collected after it exudes from the placenta. In another specific embodiment, the perfusion solution is passed through the umbilical vein and collected from the umbilical arteries, or passed through the umbilical arteries and collected from the umbilical vein.

In various embodiments, the isolated placental cells, contained within a population of cells obtained from perfusion of a placenta, are at least 50%, 60%, 70%, 80%, 90%, 95%, 99% or at least 99.5% of said population of placental cells. In another specific embodiment, the isolated placental cells collected by perfusion comprise fetal and maternal cells. In another specific embodiment, the isolated placental cells collected by perfusion are at least 50%, 60%, 70%, 80%, 90%, 95%, 99% or at least 99.5% fetal cells.

In another specific embodiment, provided herein is a composition comprising a population of the isolated placental cells, as described herein, collected by perfusion, wherein said composition comprises at least a portion of the perfusion solution used to collect the isolated placental cells.

Populations of the isolated placental cells described herein can be produced by digesting placental tissue with a tissue-disrupting enzyme to obtain a population of placental cells comprising the cells, and isolating, or substantially isolating, a plurality of the placental cells from the remainder of said placental cells. The whole, or any part of, the placenta can be digested to obtain the isolated placental cells described herein. In specific embodiments, for example, said placental tissue can be a whole placenta (e.g., including an umbilical cord), an amniotic membrane, chorion, a combination of amnion and chorion, or a combination of any of the foregoing. In other specific embodiments, the tissue-disrupting enzyme is trypsin or collagenase. In various embodiments, the isolated placental cells, contained within a population of cells obtained from digesting a placenta, are at least 50%, 60%, 70%, 80%, 90%, 95%, 99% or at least 99.5% of said population of placental cells.

The populations of isolated placental cells described above, and populations of isolated placental cells generally, can comprise about, at least, or no more than, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, 1×10¹¹ or more of the isolated placental cells. Populations of isolated placental cells useful in the methods of treatment described herein comprise at least 50%, 55%, 60%, 65%, 70%, 75%, 80%, 85%, 90%, 95%, 98%, or 99% viable isolated placental cells, e.g., as determined by, e.g., trypan blue exclusion

For any of the above placental cells, or populations of placental cells, the cells or population of placental stem cells are, or can comprise, cells that have been passaged at least 1, 2, 3, 4, 5, 6, 7, 8, 9, 10, 12, 14, 16, 18, or 20 times, or more, or expanded for 1, 2, 3, 4, 5, 6, 7, 8, 9, 10, 12, 14, 16, 18, 20, 22, 24, 26, 28, 30, 32, 34, 36, 38 or 40 population doublings, or more.

In a specific embodiment of any of the above placental cells or cell populations, the karyotype of the cells, or at least about 95% or about 99% of the cells in said population, is normal. In another specific embodiment of any of the above placental cells or cell populations, the cells, or cells in the population of cells, are non-maternal in origin.

Isolated placental cells, or populations of isolated placental cells, bearing any of the above combinations of markers, can be combined in any ratio. Any two or more of the above placental cell populations can be isolated, or enriched, to form a placental cell population. For example, an population of isolated placental cells comprising a first population of placental cells defined by one of the marker combinations described above can be combined with a second population of placental cells defined by another of the marker combinations described above, wherein said first and second populations are combined in a ratio of about 1:99, 2:98, 3:97, 4:96, 5:95, 10:90, 20:80, 30:70, 40:60, 50:50, 60:40, 70:30, 80:20, 90:10, 95:5, 96:4, 97:3, 98:2, or about 99:1. In like fashion, any three, four, five or more of the above-described placental cells or placental cell populations can be combined.

In a specific embodiment of the above-mentioned placental cells, the placental cells constitutively secrete IL-6, IL-8 and monocyte chemoattractant protein (MCP-1).

The immunosuppressive pluralities of placental cells described above can comprise about, at least, or no more than, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, 1×10¹¹ or more placental cells.

In certain embodiments, the placental cells (e.g., PDACs) useful in the methods provided herein, do not express CD34, as detected by immunolocalization, after exposure to 1 to 100 ng/mL VEGF for 4 to 21 days. In a specific embodiment, said placental adherent cells are adherent to tissue culture plastic. In another specific embodiment, said population of cells induce endothelial cells to form sprouts or tube-like structures when cultured in the presence of an angiogenic factor such as vascular endothelial growth factor (VEGF), epithelial growth factor (EGF), platelet derived growth factor (PDGF) or basic fibroblast growth factor (bFGF), e.g., on a substrate such as MATRIGEL™.

In another aspect, the PDACs provided herein, a population of cells, e.g., a population of PDACs, or a population of cells wherein at least about 50%, 60%, 70%, 80%, 90%, 95% or 98% of cells in said population of cells are PDACs, secrete one or more, or all, of VEGF, HGF, IL-8, MCP-3, FGF2, follistatin, G-CSF, EGF, ENA-78, GRO, IL-6, MCP-1, PDGF-BB, TIMP-2, uPAR, or galectin-1, e.g., into culture medium in which the cell, or cells, are grown. In another embodiment, the PDACs express increased levels of CD202b, IL-8 and/or VEGF under hypoxic conditions (e.g., less than about 5% O₂) compared to normoxic conditions (e.g., about 20% or about 21% O₂).

In another embodiment, any of the PDACs or populations of cells comprising PDACs described herein can cause the formation of sprouts or tube-like structures in a population of endothelial cells in contact with said placental derived adherent cells. In a specific embodiment, the PDACs are co-cultured with human endothelial cells, which form sprouts or tube-like structures, or support the formation of endothelial cell sprouts, e.g., when cultured in the presence of extracellular matrix proteins such as collagen type I and IV, and/or angiogenic factors such as vascular endothelial growth factor (VEGF), epithelial growth factor (EGF), platelet derived growth factor (PDGF) or basic fibroblast growth factor (bFGF), e.g., in or on a substrate such as placental collagen or MATRIGEL™ for at least 4 days. In another embodiment, any of the populations of cells comprising placental derived adherent cells, described herein, secrete angiogenic factors such as vascular endothelial growth factor (VEGF), hepatocyte growth factor (HGF), platelet derived growth factor (PDGF), basic fibroblast growth factor (bFGF), or Interleukin-8 (IL-8) and thereby can induce human endothelial cells to form sprouts or tube-like structures when cultured in the presence of extracellular matrix proteins such as collagen type I and IV e.g., in or on a substrate such as placental collagen or MATRIGEL™.

In another embodiment, any of the above populations of cells comprising placental derived adherent cells (PDACs) secretes angiogenic factors. In specific embodiments, the population of cells secretes vascular endothelial growth factor (VEGF), hepatocyte growth factor (HGF), platelet derived growth factor (PDGF), basic fibroblast growth factor (bFGF), and/or interleukin-8 (IL-8). In other specific embodiments, the population of cells comprising PDACs secretes one or more angiogenic factors and thereby induces human endothelial cells to migrate in an in vitro wound healing assay. In other specific embodiments, the population of cells comprising placental derived adherent cells induces maturation, differentiation or proliferation of human endothelial cells, endothelial progenitors, myocytes or myoblasts.

### 5.5.3 Selecting and Producing Placental Cell Populations

In certain embodiments, populations of placental cells can be selected, wherein the population is immunosuppressive. In one embodiment, for example, provided herein is a method of selecting a plurality of immunosuppressive placental cells from a plurality of placental cells, comprising selecting a population of placental cells wherein at least 10%, at least 20%, at least 30%, at least 40%, at least 50% at least 60%, at least 70%, at least 80%, at least 90%, or at least 95% of said cells are CD10⁺, CD34⁻, CD105⁺, CD200⁺ placental cells, and wherein said placental cells detectably suppresses T cell proliferation in an MLR assay. In a specific embodiment, said selecting comprises selecting stem cells that are also CD45⁻ and CD90⁺.

In another embodiment, provided herein is a method of selecting a plurality of immunosuppressive placental cells from a plurality of placental cells, comprising selecting a population of placental cells wherein at least 10%, at least 20%, at least 30%, at least 40%, at least 50% at least 60%, at least 70%, at least 80%, at least 90%, or at least 95% of said cells are CD200⁺, HLA-G⁻ placental cells, and wherein said placental cells detectably suppresses T cell proliferation in an MLR assay. In a specific embodiment, said selecting comprises selecting stem cells that are also CD73⁺ and CD105⁺. In another specific embodiment, said selecting comprises selecting stem cells that are also CD34⁻, CD38⁻ or CD45⁻. In another specific embodiment, said selecting comprises selecting placental cells that are also CD34⁻, CD38⁻, CD45⁻, CD73⁺ and CD105⁺. In another specific embodiment, said selecting also comprises selecting a plurality of placental cells that forms one or more embryoid-like bodies when cultured under conditions that allow the formation of embryoid-like bodies.

In another embodiment, provided herein is a method of selecting a plurality of immunosuppressive placental cells from a plurality of placental cells, comprising selecting a plurality of placental cells wherein at least 10%, at least 20%, at least 30%, at least 40%, at least 50% at least 60%, at least 70%, at least 80%, at least 90%, or at least 95% of said cells are CD73⁺, CD105⁺, CD200⁺ placental cells, and wherein said placental cells detectably suppresses T cell proliferation in an MLR assay. In a specific embodiment, said selecting comprises selecting stem cells that are also HLA-G⁻. In another specific embodiment, said selecting comprises selecting placental cells that are also CD34⁻, CD38⁻ or CD45⁻. In another specific embodiment, said selecting comprises selecting placental cells that are also CD34⁻, CD38⁻ and CD45⁻. In another specific embodiment, said selecting comprises selecting placental cells that are also CD34⁻, CD38⁻, CD45⁻, and HLA-G⁻. In another specific embodiment, said selecting additionally comprises selecting a population of placental cells that produces one or more embryoid-like bodies when the population is cultured under conditions that allow the formation of embryoid-like bodies.

In another embodiment, also provided herein is a method of selecting a plurality of immunosuppressive placental cells from a plurality of placental cells, comprising selecting a plurality of placental cells wherein at least 10%, at least 20%, at least 30%, at least 40%, at least 50% at least 60%, at least 70%, at least 80%, at least 90%, or at least 95% of said cells are CD200⁺, OCT-4⁺ placental cells, and wherein said placental cells detectably suppresses T cell proliferation in an MLR assay. In a specific embodiment, said selecting comprises selecting placental cells that are also CD73⁺ and CD105⁺. In another specific embodiment, said selecting comprises selecting placental cells that are also HLA-G⁻. In another specific embodiment, said selecting comprises selecting placental cells that are also CD34⁻, CD38⁻ and CD45⁻. In another specific embodiment, said selecting comprises selecting placental cells that are also CD34⁻, CD38⁻, CD45⁻, CD73⁺, CD105⁺ and HLA-G⁻.

In another embodiment, provided herein is a method of selecting a plurality of immunosuppressive placental cells from a plurality of placental cells, comprising selecting a plurality of placental cells wherein at least 10%, at least 20%, at least 30%, at least 40%, at least 50% at least 60%, at least 70%, at least 80%, at least 90%, or at least 95% of said cells are CD73⁺, CD105⁺ and HLA-G⁻ placental cells, and wherein said placental cells detectably suppresses T cell proliferation in an MLR assay. In a specific embodiment, said selecting comprises selecting placental cells that are also CD34⁻, CD38⁻ or CD45⁻. In another specific embodiment, said selecting comprises selecting placental cells that are also CD34⁻, CD38⁻ and CD45⁻. In another specific embodiment, said selecting comprises selecting placental cells that are also CD200⁺. In another specific embodiment, said selecting comprises selecting placental cells that are also CD34⁻, CD38⁻, CD45⁻, OCT-4⁺ and CD200⁺.

In another embodiment, also provided herein is provides a method of selecting a plurality of immunosuppressive placental cells from a plurality of placental cells, comprising selecting a plurality of placental cells wherein at least 10%, at least 20%, at least 30%, at least 40%, at least 50% at least 60%, at least 70%, at least 80%, at least 90%, or at least 95% of said cells are CD73⁺, CD105⁺ placental cells, and wherein said plurality forms one or more embryoid-like bodies under conditions that allow formation of embryoid-like bodies. In a specific embodiment, said selecting comprises selecting placental cells that are also CD34⁻, CD38⁻ or CD45⁻. In another specific embodiment, said selecting comprises selecting placental cells that are also CD34⁻, CD38⁻ and CD45⁻. In another specific embodiment, said selecting comprises selecting placental cells that are also OCT-4⁺. In a more specific embodiment, said selecting comprises selecting placental cells that are also OCT-4⁺, CD34⁻, CD38⁻ and CD45⁻.

In another embodiment, provided herein is a method of selecting a plurality of immunosuppressive placental cells from a plurality of placental cells, comprising selecting a plurality of placental cells wherein at least 10%, at least 20%, at least 30%, at least 40%, at least 50% at least 60%, at least 70%, at least 80%, at least 90%, or at least 95% of said isolated placental cells are OCT4⁺ stem cells, and wherein said plurality forms one or more embryoid-like bodies under conditions that allow formation of embryoid-like bodies. In a specific embodiment, said selecting comprises selecting placental cells that are also CD73⁺ and CD105⁺. In another specific embodiment, said selecting comprises selecting placental cells that are also CD34⁻, CD38⁻, or CD45⁻. In another specific embodiment, said selecting comprises selecting placental cells that are also CD200⁺. In a more specific embodiment, said selecting comprises selecting placental cells that are also CD73⁺, CD105⁺, CD200⁺, CD34⁻, CD38⁻, and CD45⁻.

Immunosuppressive populations, or pluralities, of placental cells can be produced according to the methods provided herein. For example, provided herein is method of producing a cell population, comprising selecting any of the pluralities of placental cells described above, and isolating the plurality of placental cells from other cells, e.g., other placental cells. In a specific embodiment, provided herein is a method of producing a cell population comprising selecting placental cells, wherein said placental cells (a) adhere to a substrate, (b) express CD200 and do not express HLA-G, or express CD73, CD105, and CD200, or express CD200 and OCT-4, or express CD73, CD105, and do not express HLA-G, or express CD73 and CD105 and facilitate the formation of one or more embryoid-like bodies in a population of placental cells that comprise the stem cell, when said population is cultured under conditions that allow formation of embryoid-like bodies, or express OCT-4 and facilitate the formation of one or more embryoid-like bodies in a population of placental cells that comprise the stem cell, when said population is cultured under conditions that allow formation of embryoid-like bodies; and (c) detectably suppress CD4⁺ or CD8⁺ T cell proliferation in an MLR or regression assay; and isolating said placental cells from other cells to form a cell population.

In a more specific embodiment, immunosuppressive placental cell populations can be produced by a method comprising selecting placental cells that (a) adhere to a substrate, (b) express CD200 and do not express HLA-G, and (c) detectably suppress CD4⁺ or CD8⁺ T cell proliferation in an MLR assay; and isolating said placental cells from other cells to form a cell population. In another specific embodiment, the method comprises selecting placental cells that (a) adhere to a substrate, (b) express CD73, CD105, and CD200, and (c) detectably suppress CD4⁺ or CD8⁺ T cell proliferation in an MLR; and isolating said placental cells from other cells to form a cell population. In another specific embodiment, provided herein is a method of producing a cell population comprising selecting placental cells that (a) adhere to a substrate, (b) express CD200 and OCT-4, and (c) detectably suppress CD4⁺ or CD8⁺ T cell proliferation in an MLR; and isolating said placental cells from other cells to form a cell population. In another specific embodiment, provided herein is a method of producing a cell population comprising selecting placental cells that (a) adhere to a substrate, (b) express CD73 and CD105, (c) form embryoid-like bodies when cultured under conditions allowing the formation of embryoid-like bodies, and (d) detectably suppress CD4⁺ or CD8⁺ T cell proliferation in an MLR; and isolating said placental cells from other cells to form a cell population. In another specific embodiment, the method comprises selecting placental cells that (a) adhere to a substrate, (b) express CD73 and CD105, and do not express HLA-G, and (c) detectably suppress CD4⁺ or CD8⁺ T cell proliferation in an MLR; and isolating said placental cells from other cells to form a cell population. A method of producing a cell population comprising selecting placental cells that (a) adhere to a substrate, (b) express OCT-4, (c) form embryoid-like bodies when cultured under conditions allowing the formation of embryoid-like bodies, and (d) detectably suppress CD4⁺ or CD8⁺ T cell proliferation in an MLR; and isolating said placental cells from other cells to form a cell population.

In a specific embodiment of the methods of producing an immunosuppressive placental cell population, said T cells and said placental cells are present in said MLR at a ratio of about 5:1. The placental cells used in the method can be derived from the whole placenta, or primarily from amnion, or amnion and chorion. In another specific embodiment, the placental cells suppress CD4⁺ or CD8⁺ T cell proliferation by at least 50%, at least 75%, at least 90%, or at least 95% in said MLR compared to an amount of T cell proliferation in said MLR in the absence of said placental cells. The method can additionally comprise the selection and/or production of a placental cell population capable of immunomodulation, e.g., suppression of the activity of, other immune cells, e.g., an activity of a natural killer (NK) cell.

### 5.5.4 Growth in Culture

The growth of the placental cells, e.g., the placental stem cells (PDACs) described herein, as for any mammalian cell, depends in part upon the particular medium selected for growth. Under optimum conditions, placental cells typically double in number in 3-5 days. During culture, the placental cells provided herein adhere to a substrate in culture, e.g. the surface of a tissue culture container (e.g., tissue culture dish plastic, fibronectin-coated plastic, and the like) and form a monolayer.

Populations of isolated placental cells that comprise the placental cells provided herein, when cultured under appropriate conditions, form embryoid-like bodies, that is, three-dimensional clusters of cells grow atop the adherent stem cell layer. Cells within the embryoid-like bodies express markers associated with very early stem cells, e.g., OCT-4, Nanog, SSEA3 and SSEA4. Cells within the embryoid-like bodies are typically not adherent to the culture substrate, as are the placental cells described herein, but remain attached to the adherent cells during culture. Embryoid-like body cells are dependent upon the adherent placental cells for viability, as embryoid-like bodies do not form in the absence of the adherent stem cells. The adherent placental cells thus facilitate the growth of one or more embryoid-like bodies in a population of placental cells that comprise the adherent placental cells. Without wishing to be bound by theory, the cells of the embryoid-like bodies are thought to grow on the adherent placental cells much as embryonic stem cells grow on a feeder layer of cells. Mesenchymal stem cells, e.g., bone marrow-derived mesenchymal stem cells, do not develop embryoid-like bodies in culture.

### 5.5.5 Differentiation

The placental cells, useful in the methods of treating a CNS injury, e.g., an SCI or TBI, provided herein, in certain embodiments are differentiable into different committed cell lineages. For example, in certain embodiments, the placental cells can be differentiated into cells of an adipogenic, chondrogenic, neurogenic, or osteogenic lineage. Such differentiation can be accomplished, e.g., by any method known in the art for differentiating, e.g., bone marrow-derived mesenchymal stem cells into similar cell lineages, or by methods described elsewhere herein. Specific methods of differentiating placental cells into particular cell lineages are disclosed in, e.g., U.S. Pat. No. 7,311,905, and in U.S. Patent Application Publication No. 2007/0275362, the disclosures of which are hereby incorporated by reference in their entireties.

The placental cells provided herein can exhibit the capacity to differentiate into a particular cell lineage in vitro, in vivo, or in vitro and in vivo. In a specific embodiment, the placental cells provided herein can be differentiated in vitro when placed in conditions that cause or promote differentiation into a particular cell lineage, but do not detectably differentiate in vivo, e.g., in a NOD-SCID mouse model.

### 5.6 Methods of Obtaining Placental Cells

### 5.6.1 Stem Cell Collection Composition

Placental cells can be collected and isolated according to the methods provided herein. Generally, stem cells are obtained from a mammalian placenta using a physiologically-acceptable solution, e.g., a stem cell collection composition. A stem cell collection composition is described in detail in related U.S. Provisional Application No. 60/754,969, entitled “Improved Composition for Collecting and Preserving Placental cells and Methods of Using the Composition” filed on Dec. 29, 2005.

The stem cell collection composition can comprise any physiologically-acceptable solution suitable for the collection and/or culture of stem cells, for example, a saline solution (e.g., phosphate-buffered saline, Kreb's solution, modified Kreb's solution, Eagle's solution, 0.9% NaCl. etc.), a culture medium (e.g., DMEM, HDMEM, etc.), and the like.

The stem cell collection composition can comprise one or more components that tend to preserve placental cells, that is, prevent the placental cells from dying, or delay the death of the placental cells, reduce the number of placental cells in a population of cells that die, or the like, from the time of collection to the time of culturing. Such components can be, e.g., an apoptosis inhibitor (e.g., a caspase inhibitor or JNK inhibitor); a vasodilator (e.g., magnesium sulfate, an antihypertensive drug, atrial natriuretic peptide (ANP), adrenocorticotropin, corticotropin-releasing hormone, sodium nitroprusside, hydralazine, adenosine triphosphate, adenosine, indomethacin or magnesium sulfate, a phosphodiesterase inhibitor, etc.); a necrosis inhibitor (e.g., 2-(1H-Indol-3-yl)-3-pentylamino-maleimide, pyrrolidine dithiocarbamate, or clonazepam); a TNF-α inhibitor; and/or an oxygen-carrying perfluorocarbon (e.g., perfluorooctyl bromide, perfluorodecyl bromide, etc.).

The stem cell collection composition can comprise one or more tissue-degrading enzymes, e.g., a metalloprotease, a serine protease, a neutral protease, an RNase, or a DNase, or the like. Such enzymes include, but are not limited to, collagenases (e.g., collagenase I, II, III or IV, a collagenase from Clostridium histolyticum, etc.); dispase, thermolysin, elastase, trypsin, LIBERASE, hyaluronidase, and the like.

The stem cell collection composition can comprise a bacteriocidally or bacteriostatically effective amount of an antibiotic. In certain non-limiting embodiments, the antibiotic is a macrolide (e.g., tobramycin), a cephalosporin (e.g., cephalexin, cephradine, cefuroxime, cefprozil, cefaclor, cefixime or cefadroxil), a clarithromycin, an erythromycin, a penicillin (e.g., penicillin V) or a quinolone (e.g., ofloxacin, ciprofloxacin or norfloxacin), a tetracycline, a streptomycin, etc. In a particular embodiment, the antibiotic is active against Gram(+) and/or Gram(−) bacteria, e.g., Pseudomonas aeruginosa, Staphylococcus aureus, and the like.

The stem cell collection composition can also comprise one or more of the following compounds: adenosine (about 1 mM to about 50 mM); D-glucose (about 20 mM to about 100 mM); magnesium ions (about 1 mM to about 50 mM); a macromolecule of molecular weight greater than 20,000 daltons, in one embodiment, present in an amount sufficient to maintain endothelial integrity and cellular viability (e.g., a synthetic or naturally occurring colloid, a polysaccharide such as dextran or a polyethylene glycol present at about 25 g/l to about 100 g/l, or about 40 g/l to about 60 g/l); an antioxidant (e.g., butylated hydroxyanisole, butylated hydroxytoluene, glutathione, vitamin C or vitamin E present at about 25 μM to about 100 μM); a reducing agent (e.g., N-acetylcysteine present at about 0.1 mM to about 5 mM); an agent that prevents calcium entry into cells (e.g., verapamil present at about 2 μM to about 25 μM); nitroglycerin (e.g., about 0.05 g/L to about 0.2 g/L); anticoagulant, in one embodiment, present in an amount sufficient to help prevent clotting of residual blood (e.g., heparin or hirudin present at a concentration of about 1000 units/l to about 100,000 units/1); or an amiloride containing compound (e.g., amiloride, ethyl isopropyl amiloride, hexamethylene amiloride, dimethyl amiloride or isobutyl amiloride present at about 1.0 μM to about 5 μM).

### 5.6.2 Collection and Handling of Placenta

Generally, a human placenta is recovered shortly after its expulsion after birth. In a preferred embodiment, the placenta is recovered from a patient after informed consent and after a complete medical history of the patient is taken and is associated with the placenta. Preferably, the medical history continues after delivery. Such a medical history can be used to coordinate subsequent use of the placenta or the stem cells harvested therefrom. For example, human placental cells can be used, in light of the medical history, for personalized medicine for the infant associated with the placenta, or for parents, siblings or other relatives of the infant.

Prior to recovery of placental cells, the umbilical cord blood and placental blood are removed. In certain embodiments, after delivery, the cord blood in the placenta is recovered. The placenta can be subjected to a conventional cord blood recovery process. Typically a needle or cannula is used, with the aid of gravity, to exsanguinate the placenta (see, e.g., Anderson, U.S. Pat. No. 5,372,581; Hessel et al., U.S. Pat. No. 5,415,665). The needle or cannula is usually placed in the umbilical vein and the placenta can be gently massaged to aid in draining cord blood from the placenta. Such cord blood recovery may be performed commercially, e.g., LifeBank Inc., Cedar Knolls, N.J., ViaCord, Cord Blood Registry and Cryocell. Preferably, the placenta is gravity drained without further manipulation so as to minimize tissue disruption during cord blood recovery.

Typically, a placenta is transported from the delivery or birthing room to another location, e.g., a laboratory, for recovery of cord blood and collection of stem cells by, e.g., perfusion or tissue dissociation. The placenta is preferably transported in a sterile, thermally insulated transport device (maintaining the temperature of the placenta between 20-28° C.), for example, by placing the placenta, with clamped proximal umbilical cord, in a sterile zip-lock plastic bag, which is then placed in an insulated container. In another embodiment, the placenta is transported in a cord blood collection kit substantially as described in pending U.S. patent application Ser. No. 11/230,760, filed Sep. 19, 2005. Preferably, the placenta is delivered to the laboratory four to twenty-four hours following delivery. In certain embodiments, the proximal umbilical cord is clamped, preferably within 4-5 cm (centimeter) of the insertion into the placental disc prior to cord blood recovery. In other embodiments, the proximal umbilical cord is clamped after cord blood recovery but prior to further processing of the placenta.

The placenta, prior to stem cell collection, can be stored under sterile conditions and at either room temperature or at a temperature of 5 to 25° C. (centigrade). The placenta may be stored for a period of longer than forty eight hours, and preferably for a period of four to twenty-four hours prior to perfusing the placenta to remove any residual cord blood. The placenta is preferably stored in an anticoagulant solution at a temperature of 5 to 25° C. (centigrade). Suitable anticoagulant solutions are well known in the art. For example, a solution of heparin or warfarin sodium can be used. In a preferred embodiment, the anticoagulant solution comprises a solution of heparin (e.g., 1% w/w in 1:1000 solution). The exsanguinated placenta is preferably stored for no more than 36 hours before placental cells are collected.

The mammalian placenta or a part thereof, once collected and prepared generally as above, can be treated in any art-known manner, e.g., can be perfused or disrupted, e.g., digested with one or more tissue-disrupting enzymes, to obtain stem cells.

### 5.6.3 Physical Disruption and Enzymatic Digestion of Placental Tissue

In one embodiment, stem cells are collected from a mammalian placenta by physical disruption, e.g., enzymatic digestion, of the organ, e.g., using the stem cell collection composition described in Section 5.3.1, above. For example, the placenta, or a portion thereof, may be, e.g., crushed, sheared, minced, diced, chopped, macerated or the like, while in contact with, e.g., a buffer, medium or a stem cell collection composition, and the tissue subsequently digested with one or more enzymes. The placenta, or a portion thereof, may also be physically disrupted and digested with one or more enzymes, and the resulting material then immersed in, or mixed into, a buffer, medium or a stem cell collection composition. Any method of physical disruption can be used, provided that the method of disruption leaves a plurality, more preferably a majority, and more preferably at least 60%, 70%, 80%, 90%, 95%, 98%, or 99% of the cells in said organ viable, as determined by, e.g., trypan blue exclusion.

The placenta can be dissected into components prior to physical disruption and/or enzymatic digestion and stem cell recovery. For example, placental cells can be obtained from the amniotic membrane, chorion, placental cotyledons, or any combination thereof, or umbilical cord, or any combination thereof. Preferably, placental cells are obtained from placental tissue comprising amnion and chorion, or amnion-chorion and umbilical cord. In one embodiment, stem cells are obtained from amnion-chorion and umbilical cord in about a 1:1 weight ratio. Typically, placental cells can be obtained by disruption of a small block of placental tissue, e.g., a block of placental tissue that is about 1, 2, 3, 4, 5, 6, 7, 8, 9, 10, 20, 30, 40, 50, 60, 70, 80, 90, 100, 200, 300, 400, 500, 600, 700, 800, 900 or about 1000 cubic millimeters in volume.

A preferred stem cell collection composition comprises one or more tissue-disruptive enzyme(s). Enzymatic digestion preferably uses a combination of enzymes, e.g., a combination of a matrix metalloprotease and a neutral protease, for example, a combination of collagenase and dispase. In one embodiment, enzymatic digestion of placental tissue uses a combination of a matrix metalloprotease, a neutral protease, and a mucolytic enzyme for digestion of hyaluronic acid, such as a combination of collagenase, dispase, and hyaluronidase or a combination of LIBERASE (Boehringer Mannheim Corp., Indianapolis, Ind.) and hyaluronidase. Other enzymes that can be used to disrupt placenta tissue include papain, deoxyribonucleases, serine proteases, such as trypsin, chymotrypsin, or elastase. Serine proteases may be inhibited by alpha 2 microglobulin in serum and therefore the medium used for digestion is usually serum-free. EDTA and DNase are commonly used in enzyme digestion procedures to increase the efficiency of cell recovery. The digestate is preferably diluted so as to avoid trapping stem cells within the viscous digest.

Any combination of tissue digestion enzymes can be used. Typical concentrations for tissue digestion enzymes include, e.g., 50-200 U/mL for collagenase I and collagenase IV, 1-10 U/mL for dispase, and 10-100 U/mL for elastase. Proteases can be used in combination, that is, two or more proteases in the same digestion reaction, or can be used sequentially in order to liberate placental cells. For example, in one embodiment, a placenta, or part thereof, is digested first with an appropriate amount of collagenase I at 2 mg/ml for 30 minutes, followed by digestion with trypsin, 0.25%, for 10 minutes, at 37° C. Serine proteases are preferably used consecutively following use of other enzymes.

In another embodiment, the tissue can further be disrupted by the addition of a chelator, e.g., ethylene glycol bis(2-aminoethyl ether)-N,N,N′N′-tetraacetic acid (EGTA) or ethylenediaminetetraacetic acid (EDTA) to the stem cell collection composition comprising the stem cells, or to a solution in which the tissue is disrupted and/or digested prior to isolation of the stem cells with the stem cell collection composition.

It will be appreciated that where an entire placenta, or portion of a placenta comprising both fetal and maternal cells (for example, where the portion of the placenta comprises the chorion or cotyledons), the placental cells collected will comprise a mix of placental cells derived from both fetal and maternal sources. Where a portion of the placenta that comprises no, or a negligible number of, maternal cells (for example, amnion), the placental cells collected will comprise almost exclusively fetal placental cells.

### 5.6.4 Placental Perfusion

Placental cells, e.g., placental stem cells (PDACs) can also be obtained by perfusion of the mammalian placenta. Methods of perfusing mammalian placenta to obtain stem cells are disclosed, e.g., in Hariri, U.S. Application Publication No. 2002/0123141, and in related U.S. Provisional Application No. 60/754,969, entitled “Improved Composition for Collecting and Preserving Placental cells and Methods of Using the Composition” filed on Dec. 29, 2005.

Placental cells can be collected by perfusion, e.g., through the placental vasculature, using, e.g., a stem cell collection composition as a perfusion solution. In one embodiment, a mammalian placenta is perfused by passage of perfusion solution through either or both of the umbilical artery and umbilical vein. The flow of perfusion solution through the placenta may be accomplished using, e.g., gravity flow into the placenta. Preferably, the perfusion solution is forced through the placenta using a pump, e.g., a peristaltic pump. The umbilical vein can be, e.g., cannulated with a cannula, e.g., a TEFLON® or plastic cannula, that is connected to a sterile connection apparatus, such as sterile tubing. The sterile connection apparatus is connected to a perfusion manifold.

In preparation for perfusion, the placenta is preferably oriented (e.g., suspended) in such a manner that the umbilical artery and umbilical vein are located at the highest point of the placenta. The placenta can be perfused by passage of a perfusion fluid, e.g., the stem cell collection composition provided herein, through the placental vasculature, or through the placental vasculature and surrounding tissue. In one embodiment, the umbilical artery and the umbilical vein are connected simultaneously to a pipette that is connected via a flexible connector to a reservoir of the perfusion solution. The perfusion solution is passed into the umbilical vein and artery. The perfusion solution exudes from and/or passes through the walls of the blood vessels into the surrounding tissues of the placenta, and is collected in a suitable open vessel from the surface of the placenta that was attached to the uterus of the mother during gestation. The perfusion solution may also be introduced through the umbilical cord opening and allowed to flow or percolate out of openings in the wall of the placenta which interfaced with the maternal uterine wall. In another embodiment, the perfusion solution is passed through the umbilical veins and collected from the umbilical artery, or is passed through the umbilical artery and collected from the umbilical veins.

In one embodiment, the proximal umbilical cord is clamped during perfusion, and more preferably, is clamped within 4-5 cm (centimeter) of the cord's insertion into the placental disc.

The first collection of perfusion fluid from a mammalian placenta during the exsanguination process is generally colored with residual red blood cells of the cord blood and/or placental blood. The perfusion fluid becomes more colorless as perfusion proceeds and the residual cord blood cells are washed out of the placenta. Generally from 30 to 100 ml (milliliter) of perfusion fluid is adequate to initially exsanguinate the placenta, but more or less perfusion fluid may be used depending on the observed results.

The volume of perfusion liquid used to collect placental cells may vary depending upon the number of stem cells to be collected, the size of the placenta, the number of collections to be made from a single placenta, etc. In various embodiments, the volume of perfusion liquid may be from 50 mL to 5000 mL, 50 mL to 4000 mL, 50 mL to 3000 mL, 100 mL to 2000 mL, 250 mL to 2000 mL, 500 mL to 2000 mL, or 750 mL to 2000 mL. Typically, the placenta is perfused with 700-800 mL of perfusion liquid following exsanguination.

The placenta can be perfused a plurality of times over the course of several hours or several days. Where the placenta is to be perfused a plurality of times, it may be maintained or cultured under aseptic conditions in a container or other suitable vessel, and perfused with the stem cell collection composition, or a standard perfusion solution (e.g., a normal saline solution such as phosphate buffered saline (“PBS”)) with or without an anticoagulant (e.g., heparin, warfarin sodium, coumarin, bishydroxycoumarin), and/or with or without an antimicrobial agent (e.g., β-mercaptoethanol (0.1 mM); antibiotics such as streptomycin (e.g., at 40-100 μg/ml), penicillin (e.g., at 40 U/ml), amphotericin B (e.g., at 0.5 μg/ml). In one embodiment, an isolated placenta is maintained or cultured for a period of time without collecting the perfusate, such that the placenta is maintained or cultured for 1, 2, 3, 4, 5, 6, 7, 8, 9, 10, 11, 12, 13, 14, 15, 16, 17, 18, 19, 20, 21, 22, 23, or 24 hours, or 2 or 3 or more days before perfusion and collection of perfusate. The perfused placenta can be maintained for one or more additional time(s), e.g., 1, 2, 3, 4, 5, 6, 7, 8, 9, 10, 11, 12, 13, 14, 15, 16, 17, 18, 19, 20, 21, 22, 23, 24 or more hours, and perfused a second time with, e.g., 700-800 mL perfusion fluid. The placenta can be perfused 1, 2, 3, 4, 5 or more times, for example, once every 1, 2, 3, 4, 5 or 6 hours. In a preferred embodiment, perfusion of the placenta and collection of perfusion solution, e.g., stem cell collection composition, is repeated until the number of recovered nucleated cells falls below 100 cells/ml. The perfusates at different time points can be further processed individually to recover time-dependent populations of cells, e.g., stem cells. Perfusates from different time points can also be pooled.

Without wishing to be bound by any theory, after exsanguination and a sufficient time of perfusion of the placenta, placental cells are believed to migrate into the exsanguinated and perfused microcirculation of the placenta where they are collected, preferably by washing into a collecting vessel by perfusion. Perfusing the isolated placenta not only serves to remove residual cord blood but also provide the placenta with the appropriate nutrients, including oxygen. The placenta may be cultivated and perfused with a similar solution which was used to remove the residual cord blood cells, preferably, without the addition of anticoagulant agents.

Perfusion as described herein results in the collection of significantly more placental cells than the number obtainable from a mammalian placenta not perfused with said solution, and not otherwise treated to obtain stem cells (e.g., by tissue disruption, e.g., enzymatic digestion). In this context, “significantly more” means at least 10% more. Perfusion yields significantly more placental cells than, e.g., the number of placental cells obtainable from culture medium in which a placenta, or portion thereof, has been cultured.

Stem cells can be isolated from placenta by perfusion with a solution comprising one or more proteases or other tissue-disruptive enzymes. In a specific embodiment, a placenta or portion thereof (e.g., amniotic membrane, amnion and chorion, placental lobule or cotyledon, or combination of any of the foregoing) is brought to 25-37° C., and is incubated with one or more tissue-disruptive enzymes in 200 mL of a culture medium for 30 minutes. Cells from the perfusate are collected, brought to 4° C., and washed with a cold inhibitor mix comprising 5 mM EDTA, 2 mM dithiothreitol and 2 mM beta-mercaptoethanol. The stem cells are washed after several minutes with a cold (e.g., 4° C.) stem cell collection composition described elsewhere herein.

It will be appreciated that perfusion using the pan method, that is, whereby perfusate is collected after it has exuded from the maternal side of the placenta, results in a mix of fetal and maternal cells. As a result, the cells collected by this method comprise a mixed population of placental cells of both fetal and maternal origin. In contrast, perfusion solely through the placental vasculature, whereby perfusion fluid is passed through one or two placental vessels and is collected solely through the remaining vessel(s), results in the collection of a population of placental cells almost exclusively of fetal origin.

### 5.6.5 Isolation, Sorting, and Characterization of Placental cells

Stem cells from mammalian placenta, whether obtained by perfusion or enyzmatic digestion, can initially be purified from (i.e., be isolated from) other cells by Ficoll gradient centrifugation. Such centrifugation can follow any standard protocol for centrifugation speed, etc. In one embodiment, for example, cells collected from the placenta are recovered from perfusate by centrifugation at 5000×g for 15 minutes at room temperature, which separates cells from, e.g., contaminating debris and platelets. In another embodiment, placental perfusate is concentrated to about 200 ml, gently layered over Ficoll, and centrifuged at about 1100×g for 20 minutes at 22° C., and the low-density interface layer of cells is collected for further processing.

Cell pellets can be resuspended in fresh stem cell collection composition, or a medium suitable for stem cell maintenance, e.g., IMDM serum-free medium containing 2 U/ml heparin and 2 mM EDTA (GibcoBRL, NY). The total mononuclear cell fraction can be isolated, e.g., using Lymphoprep (Nycomed Pharma, Oslo, Norway) according to the manufacturer's recommended procedure.

As used herein, “isolating” placental cells, e.g., placental stem cells (PDACs) means to remove at least 20%, 30%, 40%, 50%, 60%, 70%, 80%, 90%, 95% or 99% of the cells with which the stem cells are normally associated in the intact mammalian placenta. A stem cell from an organ is “isolated” when it is present in a population of cells that comprises fewer than 50% of the cells with which the stem cell is normally associated in the intact organ.

Placental cells obtained by perfusion or digestion can, for example, be further, or initially, isolated by differential trypsinization using, e.g., a solution of 0.05% trypsin with 0.2% EDTA (Sigma, St. Louis Mo.). Differential trypsinization is possible because placental cells (PDACs) typically detach from plastic surfaces within about five minutes whereas other adherent populations typically require more than 20-30 minutes incubation. The detached placental cells can be harvested following trypsinization and trypsin neutralization, using, e.g., Trypsin Neutralizing Solution (TNS, Cambrex). In one embodiment of isolation of adherent cells, aliquots of, for example, about 5−10×10⁶ cells are placed in each of several T-75 flasks, preferably fibronectin-coated T75 flasks. In such an embodiment, the cells can be cultured with commercially available Mesenchymal Stem Cell Growth Medium (MSCGM) (Cambrex), and placed in a tissue culture incubator (37° C., 5% CO₂). After 10 to 15 days, non-adherent cells are removed from the flasks by washing with PBS. The PBS is then replaced by MSCGM. Flasks are preferably examined daily for the presence of various adherent cell types and in particular, for identification and expansion of clusters of fibroblastoid cells.

The number and type of cells collected from a mammalian placenta can be monitored, for example, by measuring changes in morphology and cell surface markers using standard cell detection techniques such as flow cytometry, cell sorting, immunocytochemistry (e.g., staining with tissue specific or cell-marker specific antibodies) fluorescence activated cell sorting (FACS), magnetic activated cell sorting (MACS), by examination of the morphology of cells using light or confocal microscopy, and/or by measuring changes in gene expression using techniques well known in the art, such as PCR and gene expression profiling. These techniques can be used, too, to identify cells that are positive for one or more particular markers. For example, using antibodies to CD34, one can determine, using the techniques above, whether a cell comprises a detectable amount of CD34; if so, the cell is CD34⁺. Likewise, if a cell produces enough OCT-4 RNA to be detectable by RT-PCR, or significantly more OCT-4 RNA than an adult cell, the cell is OCT-4⁺. Antibodies to cell surface markers (e.g., CD markers such as CD34) and the sequence of stem cell-specific genes, such as OCT-4, are well-known in the art.

Placental cells, particularly cells that have been isolated by Ficoll separation, differential adherence, or a combination of both, may be sorted using a fluorescence activated cell sorter (FACS). Fluorescence activated cell sorting (FACS) is a well-known method for separating particles, including cells, based on the fluorescent properties of the particles (Kamarch, 1987, Methods Enzymol, 151:150-165). Laser excitation of fluorescent moieties in the individual particles results in a small electrical charge allowing electromagnetic separation of positive and negative particles from a mixture. In one embodiment, cell surface marker-specific antibodies or ligands are labeled with distinct fluorescent labels. Cells are processed through the cell sorter, allowing separation of cells based on their ability to bind to the antibodies used. FACS sorted particles may be directly deposited into individual wells of 96-well or 384-well plates to facilitate separation and cloning.

In one sorting scheme, stem cells from placenta are sorted on the basis of expression of the markers CD34, CD38, CD44, CD45, CD73, CD105, OCT-4 and/or HLA-G. This can be accomplished in connection with procedures to select stem cells on the basis of their adherence properties in culture. For example, an adherence selection stem can be accomplished before or after sorting on the basis of marker expression. In one embodiment, for example, cells are sorted first on the basis of their expression of CD34; CD34⁻ cells are retained, and cells that are CD200⁺HLA-G⁺, are separated from all other CD34⁻ cells. In another embodiment, cells from placenta are based on their expression of markers CD200 and/or HLA-G; for example, cells displaying either of these markers are isolated for further use. Cells that express, e.g., CD200 and/or HLA-G can, in a specific embodiment, be further sorted based on their expression of CD73 and/or CD105, or epitopes recognized by antibodies SH2, SH3 or SH4, or lack of expression of CD34, CD38 or CD45. For example, in one embodiment, placental cells are sorted by expression, or lack thereof, of CD200, HLA-G, CD73, CD105, CD34, CD38 and CD45, and placental cells that are CD200⁺, HLA-G⁻, CD73⁺, CD105⁺, CD34, CD38⁻ and CD45⁻ are isolated from other placental cells for further use.

In another embodiment, magnetic beads can be used to separate cells. The cells may be sorted using a magnetic activated cell sorting (MACS) technique, a method for separating particles based on their ability to bind magnetic beads (0.5-100 μm diameter). A variety of useful modifications can be performed on the magnetic microspheres, including covalent addition of antibody that specifically recognizes a particular cell surface molecule or hapten. The beads are then mixed with the cells to allow binding. Cells are then passed through a magnetic field to separate out cells having the specific cell surface marker. In one embodiment, these cells can then isolated and re-mixed with magnetic beads coupled to an antibody against additional cell surface markers. The cells are again passed through a magnetic field, isolating cells that bound both the antibodies. Such cells can then be diluted into separate dishes, such as microtiter dishes for clonal isolation.

Placental cells can also be characterized and/or sorted based on cell morphology and growth characteristics. For example, placental cells can be characterized as having, and/or selected on the basis of, e.g., a fibroblastoid appearance in culture. Placental cells can also be characterized as having, and/or be selected, on the basis of their ability to form embryoid-like bodies. In one embodiment, for example, placental cells that are fibroblastoid in shape, express CD73 and CD105, and produce one or more embryoid-like bodies in culture are isolated from other placental cells. In another embodiment, OCT-4⁺ placental cells that produce one or more embryoid-like bodies in culture are isolated from other placental cells.

In another embodiment, placental cells can be identified and characterized by a colony forming unit assay. Colony forming unit assays are commonly known in the art, such as Mesen Cult™ medium (Stem Cell Technologies, Inc., Vancouver British Columbia)

Placental cells can be assessed for viability, proliferation potential, and longevity using standard techniques known in the art, such as trypan blue exclusion assay, fluorescein diacetate uptake assay, propidium iodide uptake assay (to assess viability); and thymidine uptake assay, MTT cell proliferation assay (to assess proliferation). Longevity may be determined by methods well known in the art, such as by determining the maximum number of population doubling in an extended culture.

Placental cells can also be separated from other placental cells using other techniques known in the art, e.g., selective growth of desired cells (positive selection), selective destruction of unwanted cells (negative selection); separation based upon differential cell agglutinability in the mixed population as, for example, with soybean agglutinin; freeze-thaw procedures; filtration; conventional and zonal centrifugation; centrifugal elutriation (counter-streaming centrifugation); unit gravity separation; countercurrent distribution; electrophoresis; and the like.

### 5.7 Culture of Placental Cells

### 5.7.1 Culture Media

Isolated placental cells, or placental cell population, or cells or placental tissue from which placental cells grow out, can be used to initiate, or seed, cell cultures. Cells are generally transferred to sterile tissue culture vessels either uncoated or coated with extracellular matrix or ligands such as laminin, collagen (e.g., native or denatured), gelatin, fibronectin, ornithine, vitronectin, and extracellular membrane protein (e.g., MATRIGEL (BD Discovery Labware, Bedford, Mass.)).

Placental cells can be cultured in any medium, and under any conditions, recognized in the art as acceptable for the culture of stem cells. Preferably, the culture medium comprises serum. Placental cells can be cultured in, for example, DMEM-LG (Dulbecco's Modified Essential Medium, low glucose)/MCDB 201 (chick fibroblast basal medium) containing ITS (insulin-transferrin-selenium), LA+BSA (linoleic acid-bovine serum albumin), dextrose, L-ascorbic acid, PDGF, EGF, IGF-1, and penicillin/streptomycin; DMEM-HG (high glucose) comprising 10% fetal bovine serum (FBS); DMEM-HG comprising 15% FBS; IMDM (Iscove's modified Dulbecco's medium) comprising 10% FBS, 10% horse serum, and hydrocortisone; M199 comprising 10% FBS, EGF, and heparin; α-MEM (minimal essential medium) comprising 10% FBS, GlutaMAX™ and gentamicin; DMEM comprising 10% FBS, GlutaMAX™ and gentamicin, etc. A preferred medium is DMEM-LG/MCDB-201 comprising 2% FBS, ITS, LA+BSA, dextrose, L-ascorbic acid, PDGF, EGF, and penicillin/streptomycin.

Other media in that can be used to culture placental cells include DMEM (high or low glucose), Eagle's basal medium, Ham's F10 medium (F10), Ham's F-12 medium (F12), Iscove's modified Dulbecco's medium, Mesenchymal Stem Cell Growth Medium (MSCGM), Liebovitz's L-15 medium, MCDB, DMIEM/F12, RPMI 1640, advanced DMEM (Gibco), DMEM/MCDB201 (Sigma), and CELL-GRO FREE.

The culture medium can be supplemented with one or more components including, for example, serum (e.g., fetal bovine serum (FBS), preferably about 2-15% (v/v); equine (horse) serum (ES); human serum (HS)); beta-mercaptoethanol (BME), preferably about 0.001% (v/v); one or more growth factors, for example, platelet-derived growth factor (PDGF), epidermal growth factor (EGF), basic fibroblast growth factor (bFGF), insulin-like growth factor-1 (IGF-1), leukemia inhibitory factor (LIF), vascular endothelial growth factor (VEGF), and erythropoietin (EPO); amino acids, including L-valine; and one or more antibiotic and/or antimycotic agents to control microbial contamination, such as, for example, penicillin G, streptomycin sulfate, amphotericin B, gentamicin, and nystatin, either alone or in combination.

### 5.7.2 Expansion and Proliferation of Placental Cells

Once an isolated placental cell, or population of isolated stem cells (e.g., a stem cell or population of stem cells separated from at least 50% of the placental cells with which the stem cell or population of stem cells is normally associated in vivo), the stem cell or population of stem cells can be proliferated and expanded in vitro. For example, a population of placental cells can be cultured in tissue culture containers, e.g., dishes, flasks, multiwell plates, or the like, for a sufficient time for the stem cells to proliferate to 70-90% confluence, that is, until the stem cells and their progeny occupy 70-90% of the culturing surface area of the tissue culture container.

Placental cells can be seeded in culture vessels at a density that allows cell growth. For example, the cells may be seeded at low density (e.g., about 1,000 to about 5,000 cells/cm²) to high density (e.g., about 50,000 or more cells/cm²). In a preferred embodiment, the cells are cultured at about 0 to about 5 percent by volume CO₂ in air. In some preferred embodiments, the cells are cultured at about 2 to about 25 percent O₂ in air, preferably about 5 to about 20 percent O₂ in air. The cells preferably are cultured at about 25° C. to about 40° C., preferably 37° C. The cells are preferably cultured in an incubator. The culture medium can be static or agitated, for example, using a bioreactor. Placental cells preferably are grown under low oxidative stress (e.g., with addition of glutathione, ascorbic acid, catalase, tocopherol, N-acetylcysteine, or the like).

Once 70%-90% confluence is obtained, the cells may be passaged. For example, the cells can be enzymatically treated, e.g., trypsinized, using techniques well-known in the art, to separate them from the tissue culture surface. After removing the cells by pipetting and counting the cells, about 20,000-100,000 stem cells, preferably about 50,000 stem cells, are passaged to a new culture container containing fresh culture medium. Typically, the new medium is the same type of medium from which the stem cells were removed. Provided herein are populations of placental cells that have been passaged at least 1, 2, 3, 4, 5, 6, 7, 8, 9, 10, 12, 14, 16, 18, or 20 times, or more, and combinations of the same.

### 5.7.3 Placental Cell Populations

The methods of treatment provided herein, in certain embodiments, use populations of placental cells. Placental cell populations can be isolated directly from one or more placentas; that is, the placental cell population can be a population of placental cells, comprising placental cells, obtained from, or contained within, perfusate, or obtained from, or contained within, digestate (that is, the collection of cells obtained by enzymatic digestion of a placenta or part thereof). Isolated placental cells as described herein can also be cultured and expanded to produce placental cell populations. Populations of placental cells comprising placental cells (e.g., PDACs) can also be cultured and expanded to produce placental stem cell populations, e.g., placental cell population comprising PDACs, or population of PDACs.

Placental cell populations described herein comprise placental cells, for example, placental cells (e.g., PDACs) as described herein. In various embodiments, at least 10%, 20%, 30%, 40%, 50%, 60%, 70%, 80%, 90%, 95%, or 99% of the cells in an isolated placental cell population are placental stem cells. That is, a placental stem cell population can comprise, e.g., as much as 1%, 5%, 10%, 20%, 30%, 40%, 50%, 60%, 70%, 80%, 90% non-stem cells.

Provided herein are methods of producing isolated placental cell populations by, e.g., selecting placental stem cells, whether derived from enzymatic digestion or perfusion, that express particular markers and/or particular culture or morphological characteristics. In one embodiment, for example, a cell population can be produced by a method comprising selecting placental cells that (a) adhere to a substrate, and (b) express CD200 and do not express HLA-G; and isolating said cells from other cells to form a cell population. In another embodiment, the method of producing a cell population comprises selecting placental cells that (a) adhere to a substrate, and (b) express CD73, CD105, and CD200; and isolating said cells from other cells to form a cell population. In another embodiment, the method of producing a cell population comprises selecting placental cells that (a) adhere to a substrate and (b) express CD200 and OCT-4; and isolating said cells from other cells to form a cell population. In another embodiment, the method of producing a cell population comprises selecting placental cells that (a) adhere to a substrate, (b) express CD73 and CD105, and (c) facilitate the formation of one or more embryoid-like bodies in a population of placental cells comprising said stem cell when said population is cultured under conditions that allow for the formation of an embryoid-like body; and isolating said cells from other cells to form a cell population. In another embodiment, the method of producing a cell population comprises selecting placental cells that (a) adhere to a substrate, and (b) express CD73 and CD105, and do not express HLA-G; and isolating said cells from other cells to form a cell population. In another embodiment, the method of producing a cell population comprises selecting placental cells that (a) adhere to a substrate, (b) express OCT-4, and (c) facilitate the formation of one or more embryoid-like bodies in a population of placental cells comprising said stem cell when said population is cultured under conditions that allow for the formation of an embryoid-like body; and isolating said cells from other cells to form a cell population. In any of the above embodiments, the method can additionally comprise selecting placental cells that express ABC-p (a placenta-specific ABC transporter protein; see, e.g., Allikmets et al., Cancer Res. 58(23):5337-9 (1998)). The method can also comprise selecting cells exhibiting at least one characteristic specific to, e.g., a mesenchymal stem cell, for example, expression of CD29, expression of CD44, expression of CD90, or expression of a combination of the foregoing.

In the above embodiments, the substrate can be any surface on which culture and/or selection of cells, e.g., placental stem cells, can be accomplished. Typically, the substrate is plastic, e.g., tissue culture dish or multiwell plate plastic. Tissue culture plastic can be coated with a biomolecule, e.g., laminin or fibronectin.

Cells, e.g., placental stem cells, can be selected for a placental cell population by any means known in the art of cell selection. For example, cells can be selected using an antibody or antibodies to one or more cell surface markers, for example, in flow cytometry or FACS. Selection can be accomplished using antibodies in conjunction with magnetic beads. Antibodies that are specific for certain stem cell-related markers are known in the art. For example, antibodies to OCT-4 (Abcam, Cambridge, Mass.), CD200 (Abcam), HLA-G (Abcam), CD73 (BD Biosciences Pharmingen, San Diego, Calif.), CD105 (Abcam; BioDesign International, Saco, Me.), etc. Antibodies to other markers are also available commercially, e.g., CD34, CD38 and CD45 are available from, e.g., StemCell Technologies or BioDesign International.

The isolated placental cell population can comprise placental cells that are not stem cells, or cells that are not placental cells.

Isolated placental cell populations can be combined with one or more populations of non-stem cells or non-placental cells. For example, an isolated population of placental cells can be combined with blood (e.g., placental blood or umbilical cord blood), blood-derived stem cells (e.g., stem cells derived from placental blood or umbilical cord blood), populations of blood-derived nucleated cells, bone marrow-derived mesenchymal cells, bone-derived stem cell populations, crude bone marrow, adult (somatic) stem cells, populations of stem cells contained within tissue, cultured stem cells, populations of fully-differentiated cells (e.g., chondrocytes, fibroblasts, amniotic cells, osteoblasts, muscle cells, cardiac cells, etc.) and the like. Cells in an isolated placental cell population can be combined with a plurality of cells of another type in ratios of about 100,000,000:1, 50,000,000:1, 20,000,000:1, 10,000,000:1, 5,000,000:1, 2,000,000:1, 1,000,000:1, 500,000:1, 200,000:1, 100,000:1, 50,000:1, 20,000:1, 10,000:1, 5,000:1, 2,000:1, 1,000:1, 500:1, 200:1, 100:1, 50:1, 20:1, 10:1, 5:1, 2:1, 1:1; 1:2; 1:5; 1:10; 1:100; 1:200; 1:500; 1:1,000; 1:2,000; 1:5,000; 1:10,000; 1:20,000; 1:50,000; 1:100,000; 1:500,000; 1:1,000,000; 1:2,000,000; 1:5,000,000; 1:10,000,000; 1:20,000,000; 1:50,000,000; or about 1:100,000,000, comparing numbers of total nucleated cells in each population. Cells in an isolated placental cell population can be combined with a plurality of cells of a plurality of cell types, as well.

In one, an isolated population of placental cells is combined with a plurality of hematopoietic stem cells. Such hematopoietic stem cells can be, for example, contained within unprocessed placental, umbilical cord blood or peripheral blood; in total nucleated cells from placental blood, umbilical cord blood or peripheral blood; in an isolated population of CD34⁺ cells from placental blood, umbilical cord blood or peripheral blood; in unprocessed bone marrow; in total nucleated cells from bone marrow; in an isolated population of CD34⁺ cells from bone marrow, or the like.

### 5.8 Preservation of Placental Cells

Placental cells can be preserved, that is, placed under conditions that allow for long-term storage, or conditions that inhibit cell death by, e.g., apoptosis or necrosis.

Placental cells can be preserved using, e.g., a composition comprising an apoptosis inhibitor, necrosis inhibitor and/or an oxygen-carrying perfluorocarbon, as described in related U.S. Provisional Application No. 60/754,969, entitled “Improved Composition for Collecting and Preserving Placental cells and Methods of Using the Composition” filed on Dec. 25, 2005. In one embodiment, provided herein is a method of preserving a population of stem cells comprising contacting said population of stem cells with a stem cell collection composition comprising an inhibitor of apoptosis and an oxygen-carrying perfluorocarbon, wherein said inhibitor of apoptosis is present in an amount and for a time sufficient to reduce or prevent apoptosis in the population of stem cells, as compared to a population of stem cells not contacted with the inhibitor of apoptosis. In a specific embodiment, said inhibitor of apoptosis is a caspase inhibitor. In another specific embodiment, said inhibitor of apoptosis is a INK inhibitor. In a more specific embodiment, said JNK inhibitor does not modulate differentiation or proliferation of said stem cells. In another embodiment, said stem cell collection composition comprises said inhibitor of apoptosis and said oxygen-carrying perfluorocarbon in separate phases. In another embodiment, said stem cell collection composition comprises said inhibitor of apoptosis and said oxygen-carrying perfluorocarbon in an emulsion. In another embodiment, the stem cell collection composition additionally comprises an emulsifier, e.g., lecithin. In another embodiment, said apoptosis inhibitor and said perfluorocarbon are between about 0° C. and about 25° C. at the time of contacting the stem cells. In another more specific embodiment, said apoptosis inhibitor and said perfluorocarbon are between about 2° C. and 10° C., or between about 2° C. and about 5° C., at the time of contacting the stem cells. In another more specific embodiment, said contacting is performed during transport of said population of stem cells. In another more specific embodiment, said contacting is performed during freezing and thawing of said population of stem cells.

In another embodiment, populations of placental cells can be preserved by a method comprising contacting said population of stem cells with an inhibitor of apoptosis and an organ-preserving compound, wherein said inhibitor of apoptosis is present in an amount and for a time sufficient to reduce or prevent apoptosis in the population of stem cells, as compared to a population of stem cells not contacted with the inhibitor of apoptosis. In a specific embodiment, the organ-preserving compound is UW solution (described in U.S. Pat. No. 4,798,824; also known as ViaSpan; see also Southard et al., Transplantation 49(2):251-257 (1990)) or a solution described in Stern et al., U.S. Pat. No. 5,552,267. In another embodiment, said organ-preserving compound is hydroxyethyl starch, lactobionic acid, raffinose, or a combination thereof. In another embodiment, the stem cell collection composition additionally comprises an oxygen-carrying perfluorocarbon, either in two phases or as an emulsion.

In another embodiment of the method, placental cells are contacted with a stem cell collection composition comprising an apoptosis inhibitor and oxygen-carrying perfluorocarbon, organ-preserving compound, or combination thereof, during perfusion. In another embodiment, said stem cells are contacted during a process of tissue disruption, e.g., enzymatic digestion. In another embodiment, placental cells are contacted with said stem cell collection compound after collection by perfusion, or after collection by tissue disruption, e.g., enzymatic digestion.

Typically, during placental cell collection, enrichment and isolation, it is preferable to minimize or eliminate cell stress due to hypoxia and mechanical stress. In another embodiment of the method, therefore, a stem cell, or population of stem cells, is exposed to a hypoxic condition during collection, enrichment or isolation for less than six hours during said preservation, wherein a hypoxic condition is a concentration of oxygen that is less than normal blood oxygen concentration. In a more specific embodiment, said population of stem cells is exposed to said hypoxic condition for less than two hours during said preservation. In another more specific embodiment, said population of stem cells is exposed to said hypoxic condition for less than one hour, or less than thirty minutes, or is not exposed to a hypoxic condition, during collection, enrichment or isolation. In another specific embodiment, said population of stem cells is not exposed to shear stress during collection, enrichment or isolation.

The placental cells described herein can be cryopreserved, e.g., in cryopreservation medium in small containers, e.g., ampoules. Suitable cryopreservation medium includes, but is not limited to, culture medium including, e.g., growth medium, or cell freezing medium, for example commercially available cell freezing medium, e.g., C2695, C2639 or C6039 (Sigma). Cryopreservation medium preferably comprises DMSO (dimethylsulfoxide), at a concentration of, e.g., about 10% (v/v). Cryopreservation medium may comprise additional agents, for example, Plasmalyte, methylcellulose with or without glycerol. Placental cells are preferably cooled at about 1° C./min during cryopreservation. A preferred cryopreservation temperature is about −80° C. to about −180° C., preferably about −125° C. to about −140° C. Cryopreserved cells can be transferred to liquid nitrogen prior to thawing for use. In some embodiments, for example, once the ampoules have reached about −90° C., they are transferred to a liquid nitrogen storage area. Cryopreserved cells preferably are thawed at a temperature of about 25° C. to about 40° C., preferably to a temperature of about 37° C.

### 5.9 Uses of Placental Cells

### 5.9.1 Compositions Comprising Placental Cells

The methods of immunosuppression provided herein can use compositions comprising placental cells, or biomolecules therefrom. In the same manner, the pluralities and populations of placental cells provided herein can be combined with any physiologically-acceptable or medically-acceptable compound, composition or device for use in, e.g., research or therapeutics.

### 5.9.1.1 Cryopreserved Placental Cells

The immunosuppressive placental cells, and populations of the cells, described herein can be preserved, for example, cryopreserved for later use. Methods for cryopreservation of cells, such as stem cells, are well known in the art. Placental cell populations can be prepared in a form that is easily administrable to an individual. For example, placental cells, or populations of the placental cells, described herein can be contained within a container that is suitable for medical use. Such a container can be, for example, a sterile plastic bag, flask, jar, or other container from which the placental cell population can be easily dispensed. For example, the container can be a blood bag or other plastic, medically-acceptable bag suitable for the intravenous administration of a liquid to a recipient. The container is preferably one that allows for cryopreservation of the combined stem cell population.

Cryopreserved immunosuppressive placental cell populations can comprise placental cells derived from a single donor, or from multiple donors. The placental cell population can be completely HLA-matched to an intended recipient, or partially or completely HLA-mismatched.

Thus, in one embodiment, provided herein is a composition comprising an immunosuppressive placental cell population in a container. In a specific embodiment, the stem cell population is cryopreserved. In another specific embodiment, the container is a bag, flask, or jar. In more specific embodiment, said bag is a sterile plastic bag. In a more specific embodiment, said bag is suitable for, allows or facilitates intravenous administration of said placental cell population. The bag can comprise multiple lumens or compartments that are interconnected to allow mixing of the placental cells and one or more other solutions, e.g., a drug, prior to, or during, administration. In another specific embodiment, the composition comprises one or more compounds that facilitate cryopreservation of the combined stem cell population. In another specific embodiment, said placental cell population is contained within a physiologically-acceptable aqueous solution. In a more specific embodiment, said physiologically-acceptable aqueous solution is a 0.9% NaCl solution. In another specific embodiment, said placental cell population comprises placental cells that are HLA-matched to a recipient of said stem cell population. In another specific embodiment, said combined stem cell population comprises placental cells that are at least partially HLA-mismatched to a recipient of said stern cell population. In another specific embodiment, said placental cells are derived from a plurality of donors.

### 5.9.1.2 Pharmaceutical Compositions

Immunosuppressive populations of placental cells, or populations of cells comprising placental cells, can be formulated into pharmaceutical compositions for use in vivo. Such pharmaceutical compositions comprise a population of placental cells, or a population of cells comprising placental cells, in a pharmaceutically-acceptable carrier, e.g., a saline solution or other accepted physiologically-acceptable solution for in vivo administration. Pharmaceutical compositions provided herein can comprise any of the placental cell populations, or placental cell types, described elsewhere herein. The pharmaceutical compositions can comprise fetal, maternal, or both fetal and maternal placental cells. The pharmaceutical compositions provided herein can further comprise placental cells obtained from a single individual or placenta, or from a plurality of individuals or placentae.

The pharmaceutical compositions provided herein can comprise any immunosuppressive number of placental cells. For example, a single unit dose of placental cells can comprise, in various embodiments, about, at least, or no more than 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, 1×10¹¹ or more placental cells.

The pharmaceutical compositions provided herein can comprise populations of cells that comprise 50% viable cells or more (that is, at least 50% of the cells in the population are functional or living). Preferably, at least 60% of the cells in the population are viable. More preferably, at least 70%, 80%, 90%, 95%, or 99% of the cells in the population in the pharmaceutical composition are viable.

The pharmaceutical compositions provided herein can comprise one or more compounds that, e.g., facilitate engraftment (e.g., anti-T-cell receptor antibodies, an immunosuppressant, or the like); stabilizers such as albumin, dextran 40, gelatin, hydroxyethyl starch, and the like.

### 5.9.1.3 Placental Cell Conditioned Media

The placental cells provided herein can be used to produce conditioned medium that is immunosuppressive, that is, medium comprising one or more biomolecules secreted or excreted by the stem cells that have a detectable immunosuppressive effect on a plurality of one or more types of immune cells. In various embodiments, the conditioned medium comprises medium in which placental cells have grown for at least 1, 2, 3, 4, 5, 6, 7, 8, 9, 10, 11, 12, 13, 14 or more days. In other embodiments, the conditioned medium comprises medium in which placental cells have grown to at least 30%, 40%, 50%, 60%, 70%, 80%, 90% confluence, or up to 100% confluence. Such conditioned medium can be used to support the culture of a separate population of placental cells, or stem cells of another kind. In another embodiment, the conditioned medium comprises medium in which placental cells have been differentiated into an adult cell type. In another embodiment, the conditioned medium comprises medium in which placental cells and non-placental cells have been cultured.

Thus, in one embodiment, provided herein is a composition comprising culture medium from a culture of placental cells, wherein said placental cells (a) adhere to a substrate; (b) express CD200 and do not express HLA-G, or express CD73, CD105, and CD200, or express CD200 and OCT-4, or express CD73 and CD105, and do not express HLA-G, or express CD73 and CD105 and facilitate the formation of one or more embryoid-like bodies in a population of placental cells that comprise the placental cells, when said population is cultured under conditions that allow formation of embryoid-like bodies, or express OCT-4 and facilitate the formation of one or more embryoid-like bodies in a population of placental cells that comprise the placental cells when said population is cultured under conditions that allow formation of embryoid-like bodies; and (c) detectably suppress CD4⁺ or CD8⁺ T cell proliferation in an MLR assay, wherein said culture of placental cells has been cultured in said medium for 24 hours or more. In a specific embodiment, the composition further comprises a plurality of said placental cells. In another specific embodiment, the composition comprises a plurality of non-placental cells. In a more specific embodiment, said non-placental cells comprise CD34⁺ cells, e.g., hematopoietic progenitor cells, such as peripheral blood hematopoietic progenitor cells, cord blood hematopoietic progenitor cells, or placental blood hematopoietic progenitor cells. The non-placental cells can also comprise other stem cells, such as mesenchymal stem cells, e.g., bone marrow-derived mesenchymal stem cells. The non-placental cells can also be one or more types of adult cells or cell lines. In another specific embodiment, the composition comprises an anti-proliferative agent, e.g., an anti-MIP-1α or anti-MIP-1β antibody.

### 5.9.1.4 Matrices Comprising Placental Cells

Further provided herein are matrices, hydrogels, scaffolds, and the like that comprise immunosuppressive placental cells, e.g., an immunosuppressive population of placental stem cells (e.g., PDACs).

Placental cells provided herein can be seeded onto a natural matrix, e.g., a placental biomaterial such as an amniotic membrane material. Such an amniotic membrane material can be, e.g., amniotic membrane dissected directly from a mammalian placenta; fixed or heat-treated amniotic membrane, substantially dry (i.e., <20% H₂O) amniotic membrane, chorionic membrane, substantially dry chorionic membrane, substantially dry amniotic and chorionic membrane, and the like. Preferred placental biomaterials on which placental cells can be seeded are described in Hariri, U.S. Application Publication No. 2004/0048796.

Placental cells provided herein can be suspended in a hydrogel solution suitable for, e.g., injection. Suitable hydrogels for such compositions include self-assembling peptides, such as RAD16. In one embodiment, a hydrogel solution comprising the cells can be allowed to harden, for instance in a mold, to form a matrix having cells dispersed therein for implantation. Placental cells in such a matrix can also be cultured so that the cells are mitotically expanded prior to implantation. The hydrogel is, e.g., an organic polymer (natural or synthetic) that is cross-linked via covalent, ionic, or hydrogen bonds to create a three-dimensional open-lattice structure that entraps water molecules to form a gel. Hydrogel-forming materials include polysaccharides such as alginate and salts thereof, peptides, polyphosphazines, and polyacrylates, which are crosslinked ionically, or block polymers such as polyethylene oxide-polypropylene glycol block copolymers which are crosslinked by temperature or pH, respectively. In some embodiments, the hydrogel or matrix is biodegradable.

In some embodiments, the formulation comprises an in situ polymerizable gel (see., e.g., U.S. Patent Application Publication 2002/0022676; Anseth et al., J. Control Release, 78(1-3):199-209 (2002); Wang et al., Biomaterials, 24(22):3969-80 (2003).

In some embodiments, the polymers are at least partially soluble in aqueous solutions, such as water, buffered salt solutions, or aqueous alcohol solutions, that have charged side groups, or a monovalent ionic salt thereof. Examples of polymers having acidic side groups that can be reacted with cations are poly(phosphazenes), poly(acrylic acids), poly(methacrylic acids), copolymers of acrylic acid and methacrylic acid, poly(vinyl acetate), and sulfonated polymers, such as sulfonated polystyrene. Copolymers having acidic side groups formed by reaction of acrylic or methacrylic acid and vinyl ether monomers or polymers can also be used. Examples of acidic groups are carboxylic acid groups, sulfonic acid groups, halogenated (preferably fluorinated) alcohol groups, phenolic OH groups, and acidic OH groups.

The placental cells or co-cultures thereof can be seeded onto a three-dimensional framework or scaffold and implanted in vivo. Such a framework can be implanted in combination with any one or more growth factors, cells, drugs or other components that stimulate tissue formation or otherwise enhance or improve the practice of the methods of treatment described elsewhere herein.

Examples of scaffolds that can be used in the methods of treatment described herein include nonwoven mats, porous foams, or self assembling peptides. Nonwoven mats can be formed using fibers comprised of a synthetic absorbable copolymer of glycolic and lactic acids (e.g., PGA/PLA) (VICRYL, Ethicon, Inc., Somerville, N.J.). Foams, composed of, e.g., poly(ε-caprolactone)/poly(glycolic acid) (PCL/PGA) copolymer, formed by processes such as freeze-drying, or lyophilization (see, e.g., U.S. Pat. No. 6,355,699), can also be used as scaffolds.

In another embodiment, the scaffold is, or comprises, a nanofibrous scaffold, e.g., an electrospun nanofibrous scaffold. In a more specific embodiment, said nanofibrous scaffold comprises poly(L-lactic acid) (PLLA), type I collagen, a copolymer of vinylidene fluoride and trifluoroethylnee (PVDF-TrFE), poly(-caprolactone), poly(L-lactide-co-ε-caprolactone) [P(LLA-CL)] (e.g., 75:25), and/or a copolymer of poly(3-hydroxybutyrate-co-3-hydroxyvalerate) (PHBV) and type I collagen. In another more specific embodiment, said scaffold promotes the differentiation of placental cells into chondrocytes. Methods of producing nanofibrous scaffolds, e.g., electrospun nanofibrous scaffolds, are known in the art. See, e.g., Xu et al., Tissue Engineering 10(7):1160-1168 (2004); Xu et al., Biomaterials 25:877-886 (20040; Meng et al., J. Biomaterials Sci., Polymer Edition 18(1):81-94 (2007).

Placental cells described herein, e.g., immunosuppressive placental cells, can also be seeded onto, or contacted with, a physiologically-acceptable ceramic material including, but not limited to, mono-, di-, tri-, alpha-tri-, beta-tri-, and tetra-calcium phosphate, hydroxyapatite, fluoroapatites, calcium sulfates, calcium fluorides, calcium oxides, calcium carbonates, magnesium calcium phosphates, biologically active glasses such as BIOGLASS®, and mixtures thereof. Porous biocompatible ceramic materials currently commercially available include SURGIBONE® (CanMedica Corp., Canada), ENDOBON® (Merck Biomaterial France, France), CEROS® (Mathys, A G, Bettlach, Switzerland), and mineralized collagen bone grafting products such as HEALOS™ (DePuy, Inc., Raynham, Mass.) and VITOSS®, RHAKOSS™, and CORTOSS® (Orthovita, Malvern, Pa.). The framework can be a mixture, blend or composite of natural and/or synthetic materials.

In another embodiment, placental cells can be seeded onto, or contacted with, a felt, which can be, e.g., composed of a multifilament yarn made from a bioabsorbable material such as PGA, PLA, PCL copolymers or blends, or hyaluronic acid.

The placental cells described herein can, in another embodiment, be seeded onto foam scaffolds that may be composite structures. Such foam scaffolds can be molded into a useful shape, such as that of a portion of a specific structure in the body to be repaired, replaced or augmented. In some embodiments, the framework is treated, e.g., with 0.1M acetic acid followed by incubation in polylysine, PBS, and/or collagen, prior to inoculation of the immunosuppressive placental cells in order to enhance cell attachment. External surfaces of a matrix may be modified to improve the attachment or growth of cells and differentiation of tissue, such as by plasma-coating the matrix, or addition of one or more proteins (e.g., collagens, elastic fibers, reticular fibers), glycoproteins, glycosaminoglycans (e.g., heparin sulfate, chondroitin-4-sulfate, chondroitin-6-sulfate, dermatan sulfate, keratin sulfate, etc.), a cellular matrix, and/or other materials such as, but not limited to, gelatin, alginates, agar, agarose, and plant gums, and the like.

In some embodiments, the scaffold comprises, or is treated with, materials that render it non-thrombogenic. These treatments and materials may also promote and sustain endothelial growth, migration, and extracellular matrix deposition. Examples of these materials and treatments include but are not limited to natural materials such as basement membrane proteins such as laminin and Type IV collagen, synthetic materials such as EPTFE, and segmented polyurethaneurea silicones, such as PURSPAN™ (The Polymer Technology Group, Inc., Berkeley, Calif.). The scaffold can also comprise anti-thrombotic agents such as heparin; the scaffolds can also be treated to alter the surface charge (e.g., coating with plasma) prior to seeding with placental cells.

### 5.9.2 Genetically Modified Placental cells

In another aspect, provided herein are placental cells that are genetically modified, e.g., to produce a nucleic acid or polypeptide of interest. Genetic modification can be accomplished, e.g., using virus-based vectors including, but not limited to, non-integrating replicating vectors, e.g., papilloma virus vectors, SV40 vectors, adenoviral vectors; integrating viral vectors, e.g., retrovirus vector or adeno-associated viral vectors; or replication-defective viral vectors. Other methods of introducing DNA into cells include the use of liposomes, electroporation, a particle gun, direct DNA injection, or the like.

Stem cells can be, e.g., transformed or transfected with DNA controlled by or in operative association with, one or more appropriate expression control elements, for example, promoter or enhancer sequences, transcription terminators, polyadenylation sites, internal ribosomal entry sites. Preferably, such a DNA incorporates a selectable marker. Following the introduction of the foreign DNA, engineered stem cells can be, e.g., grown in enriched media and then switched to selective media. In one embodiment, the DNA used to engineer a placental cell comprises a nucleotide sequence encoding a polypeptide of interest, e.g., a cytokine, growth factor, differentiation agent, or therapeutic polypeptide.

The DNA used to engineer the stem cell can comprise any promoter known in the art to drive expression of a nucleotide sequence in mammalian cells, e.g., human cells. For example, promoters include, but are not limited to, CMV promoter/enhancer, SV40 promoter, papillomavirus promoter, Epstein-Barr virus promoter, elastin gene promoter, and the like. In a specific embodiment, the promoter is regulatable so that the nucleotide sequence is expressed only when desired. Promoters can be either inducible (e.g., those associated with metallothionein and heat shock proteins) or constitutive.

In another specific embodiment, the promoter is tissue-specific or exhibits tissue specificity. Examples of such promoters include but are not limited to: myelin basic protein gene control region (Readhead et al., 1987, Cell 48:703) (oligodendrocyte cells); elastase I gene control region (Swit et al., 1984, Cell 38:639; Ornitz et al., 1986, Cold Spring Harbor Symp. Quant. Biol. 50:399; MacDonald, 1987, Hepatology 7:425) (pancreatic acinar cells); insulin gene control region (Hanahan, 1985, Nature 315:115) (pancreatic beta cells); myosin light chain-2 gene control region (Shani, 1985, Nature 314:283) (skeletal muscle).

Placental cells may be engineered to “knock out” or “knock down” expression of one or more genes. The expression of a gene native to a cell can be diminished by, for example, inhibition of expression by inactivating the gene completely by, e.g., homologous recombination. In one embodiment, for example, an exon encoding an important region of the protein, or an exon 5′ to that region, is interrupted by a positive selectable marker, e.g., neo, preventing the production of normal mRNA from the target gene and resulting in inactivation of the gene. A gene may also be inactivated by creating a deletion in part of a gene or by deleting the entire gene. By using a construct with two regions of homology to the target gene that are far apart in the genome, the sequences intervening the two regions can be deleted (Mombaerts et al., 1991, Proc. Nat. Acad. Sci. U.S.A. 88:3084). Antisense, DNAzymes, small interfering RNA, and ribozyme molecules that inhibit expression of the target gene can also be used to reduce the level of target gene activity in the stem cells. For example, antisense RNA molecules which inhibit the expression of major histocompatibility gene complexes (HLA) have been shown to be most versatile with respect to immune responses. Triple helix molecules can be utilized in reducing the level of target gene activity. See, e.g., L. G. Davis et al. (eds), 1994, BASIC METHODS IN MOLECULAR BIOLOGY, 2nd ed., Appleton & Lange, Norwalk, Conn., which is incorporated herein by reference.

In a specific embodiment, placental cells can be genetically modified with a nucleic acid molecule comprising a nucleotide sequence encoding a polypeptide of interest, wherein expression of the polypeptide of interest is controllable by an exogenous factor, e.g., polypeptide, small organic molecule, or the like. Such a polypeptide can be a therapeutic polypeptide. In a more specific embodiment, the polypeptide of interest is IL-12 or interleukin-1 receptor antagonist (IL-1Ra). In another more specific embodiment, the polypeptide of interest is a fusion of interleukin-1 receptor antagonist and dihydrofolate reductase (DHFR), and the exogenous factor is an antifolate, e.g., methotrexate. Such a construct is useful in the engineering of placental cells that express IL-1Ra, or a fusion of IL-1Ra and DHFR, upon contact with methotrexate. Such a construct can be used, e.g., in the treatment of rheumatoid arthritis. In this embodiment, the fusion of IL-1Ra and DHFR is translationally upregulated upon exposure to an antifolate such as methotrexate. Therefore, in another specific embodiment, the nucleic acid used to genetically engineer a placental cell can comprise nucleotide sequences encoding a first polypeptide and a second polypeptide, wherein said first and second polypeptides are expressed as a fusion protein that is translationally upregulated in the presence of an exogenous factor. The polypeptide can be expressed transiently or long-term (e.g., over the course of weeks or months).

Such a nucleic acid molecule can additionally comprise a nucleotide sequence encoding a polypeptide that allows for positive selection of engineered stem cells, or allows for visualization of the engineered stem cells. In another more specific embodiment, the nucleotide sequence encodes a polypeptide that is, e.g., fluorescent under appropriate visualization conditions, e.g., luciferase (Luc). In a more specific embodiment, such a nucleic acid molecule can comprise IL-1Ra-DHFR-IRES-Luc, where IRES is an internal ribosomal entry site.

### 5.9.3 Immortalized Placental Cell Lines

Mammalian placental cells can be conditionally immortalized by transfection with any suitable vector containing a growth-promoting gene, that is, a gene encoding a protein that, under appropriate conditions, promotes growth of the transfected cell, such that the production and/or activity of the growth-promoting protein is regulatable by an external factor. In a preferred embodiment the growth-promoting gene is an oncogene such as, but not limited to, v-myc, N-myc, c-myc, p53, SV40 large T antigen, polyoma large T antigen, E1a adenovirus or E7 protein of human papillomavirus.

External regulation of the growth-promoting protein can be achieved by placing the growth-promoting gene under the control of an externally-regulatable promoter, e.g., a promoter the activity of which can be controlled by, for example, modifying the temperature of the transfected cells or the composition of the medium in contact with the cells. in one embodiment, a tetracycline (tet)-controlled gene expression system can be employed (see Gossen et al., Proc. Natl. Acad. Sci. USA 89:5547-5551, 1992; Hoshimaru et al., Proc. Natl. Acad. Sci. USA 93:1518-1523, 1996). In the absence of tet, a tet-controlled transactivator (tTA) within this vector strongly activates transcription from ph_{CMV*-1}, a minimal promoter from human cytomegalovirus fused to tet operator sequences. tTA is a fusion protein of the repressor (tetR) of the transposon-10-derived tet resistance operon of Escherichia coli and the acidic domain of VP16 of herpes simplex virus. Low, non-toxic concentrations of tet (e.g., 0.01-1.0 μg/mL) almost completely abolish transactivation by tTA.

In one embodiment, the vector further contains a gene encoding a selectable marker, e.g., a protein that confers drug resistance. The bacterial neomycin resistance gene (neo^{R}) is one such marker that may be employed within the methods described herein. Cells carrying neo^{R} may be selected by means known to those of ordinary skill in the art, such as the addition of, e.g., 100-200 μg/mL G418 to the growth medium.

Transfection can be achieved by any of a variety of means known to those of ordinary skill in the art including, but not limited to, retroviral infection. In general, a cell culture may be transfected by incubation with a mixture of conditioned medium collected from the producer cell line for the vector and DMEM/F12 containing N2 supplements. For example, a placental cell culture prepared as described above may be infected after, e.g., five days in vitro by incubation for about 20 hours in one volume of conditioned medium and two volumes of DMEM/F12 containing N2 supplements. Transfected cells carrying a selectable marker may then be selected as described above.

Following transfection, cultures are passaged onto a surface that permits proliferation, e.g., allows at least 30% of the cells to double in a 24 hour period. Preferably, the substrate is a polyornithine/laminin substrate, consisting of tissue culture plastic coated with polyornithine (10 μg/mL) and/or laminin (10 μg/mL), a polylysine/laminin substrate or a surface treated with fibronectin. Cultures are then fed every 3-4 days with growth medium, which may or may not be supplemented with one or more proliferation-enhancing factors. Proliferation-enhancing factors may be added to the growth medium when cultures are less than 50% confluent.

The conditionally-immortalized placental cell lines can be passaged using standard techniques, such as by trypsinization, when 80-95% confluent. Up to approximately the twentieth passage, it is, in some embodiments, beneficial to maintain selection (by, for example, the addition of G418 for cells containing a neomycin resistance gene). Cells may also be frozen in liquid nitrogen for long-term storage.

Clonal cell lines can be isolated from a conditionally-immortalized human placental cell line prepared as described above. In general, such clonal cell lines may be isolated using standard techniques, such as by limit dilution or using cloning rings, and expanded. Clonal cell lines may generally be fed and passaged as described above.

Conditionally-immortalized human placental cell lines, which may, but need not, be clonal, may generally be induced to differentiate by suppressing the production and/or activity of the growth-promoting protein under culture conditions that facilitate differentiation. For example, if the gene encoding the growth-promoting protein is under the control of an externally-regulatable promoter, the conditions, e.g., temperature or composition of medium, may be modified to suppress transcription of the growth-promoting gene. For the tetracycline-controlled gene expression system discussed above, differentiation can be achieved by the addition of tetracycline to suppress transcription of the growth-promoting gene. In general, 1 μg/mL tetracycline for 4-5 days is sufficient to initiate differentiation. To promote further differentiation, additional agents may be included in the growth medium.

### 5.9.4 Assays

Placental cells can be used in assays to determine the influence of culture conditions, environmental factors, molecules (e.g., biomolecules, small inorganic molecules. etc.) and the like on stem cell proliferation, expansion, and/or differentiation, compared to placental cells not exposed to such conditions.

In one embodiment, placental cells can be assayed for changes in proliferation, expansion or differentiation upon contact with a molecule. In one embodiment, for example, provided herein is a method of identifying a compound that modulates the proliferation of a plurality of placental cells, comprising contacting said plurality of stem cells with said compound under conditions that allow proliferation, wherein if said compound causes a detectable change in proliferation of said plurality of stem cells compared to a plurality of stem cells not contacted with said compound, said compound is identified as a compound that modulates proliferation of placental cells. In a specific embodiment, said compound is identified as an inhibitor of proliferation. In another specific embodiment, said compound is identified as an enhancer of proliferation.

In another embodiment, compounds can be identified that modulate the expansion of a plurality of placental cells, comprising contacting said plurality of stem cells with said compound under conditions that allow expansion, wherein if said compound causes a detectable change in expansion of said plurality of stem cells compared to a plurality of stem cells not contacted with said compound, said compound is identified as a compound that modulates expansion of placental cells. In a specific embodiment, said compound is identified as an inhibitor of expansion. In another specific embodiment, said compound is identified as an enhancer of expansion.

In another embodiment, a compound that modulates the differentiation of a placental cell can be identified by a method comprising contacting said stem cells with said compound under conditions that allow differentiation, wherein if said compound causes a detectable change in differentiation of said stem cells compared to a stem cell not contacted with said compound, said compound is identified as a compound that modulates proliferation of placental cells. In a specific embodiment, said compound is identified as an inhibitor of differentiation. In another specific embodiment, said compound is identified as an enhancer of differentiation.

### 5.9.5 Placental Cell Bank

Stem cells from postpartum placentas can be cultured in a number of different ways to produce a set of lots, e.g., a set of individually-administrable doses, of placental cells. Such lots can, for example, be obtained from stem cells from placental perfusate or from enzyme-digested placental tissue. Sets of lots of placental cells, obtained from a plurality of placentas, can be arranged in a bank of placental cells for, e.g., long-term storage. Generally, adherent stem cells are obtained from an initial culture of placental material to form a seed culture, which is expanded under controlled conditions to form populations of cells from approximately equivalent numbers of doublings. Lots are preferably derived from the tissue of a single placenta, but can be derived from the tissue of a plurality of placentas.

In one embodiment, stem cell lots are obtained as follows. Placental tissue is first disrupted, e.g., by mincing, digested with a suitable enzyme, e.g., collagenase (see Section 5.3.3, above). The placental tissue preferably comprises, e.g., the entire amnion, entire chorion, or both, from a single placenta, but can comprise only a part of either the amnion or chorion. The digested tissue is cultured, e.g., for about 1-3 weeks, preferably about 2 weeks. After removal of non-adherent cells, high-density colonies that form are collected, e.g., by trypsinization. These cells are collected and resuspended in a convenient volume of culture medium, and are then used to seed expansion cultures. Expansion cultures can be any arrangement of separate cell culture apparatuses, e.g., a Cell Factory by NUNC™ Cells can be subdivided to any degree so as to seed expansion cultures with, e.g., 1×10³, 2×10³, 3×10³, 4×10³, 5×10³, 6×10³, 7×10³, 8×10³, 9×10³, 1×10⁴, 1×10⁴, 2×10⁴, 3×10⁴, 4×10⁴, 5×10⁴, 6×10⁴, 7×10⁴, 8×10⁴, 9×10⁴, or 10×10⁴ stem cells/cm². Preferably, from about 1×10³ to about 1×10⁴ cells/cm² are used to seed each expansion culture. The number of expansion cultures may be greater or fewer in number depending upon the particular placenta(s) from which the stem cells are obtained.

Expansion cultures are grown until the density of cells in culture reaches a certain value, e.g., about 1×10⁵ cells/cm². Cells can either be collected and cryopreserved at this point, or passaged into new expansion cultures as described above. Cells can be passaged, e.g., 2, 3, 4, 5, 6, 7, 8, 9, 10, 11, 12, 13, 14, 15, 16, 17, 18, 19 or 20 times prior to use. A record of the cumulative number of population doublings is preferably maintained during expansion culture(s). The cells from a culture can be expanded for 2, 3, 4, 5, 6, 7, 8, 9, 10, 12, 14, 16, 18, 20, 22, 24, 26, 28, 30, 32, 34, 36, 38 or 40 doublings, or up to 60 doublings. Preferably, however, the number of population doublings, prior to dividing the population of cells into individual doses, is from about 15 to about 30. The cells can be culture continuously throughout the expansion process, or can be frozen at one or more points during expansion.

Cells to be used for individual doses can be frozen, e.g., cryopreserved for later use. Individual doses can comprise, e.g., about 1 million to about 50 million cells per ml, and can comprise between about 10⁶ and about 10¹⁰ cells in total.

In one embodiment, therefore, a placental stem cell bank can be made by a method comprising: expanding primary culture placental stem cells from a human post-partum placenta for a first plurality of population doublings; cryopreserving said placental stem cells to form a Master Cell Bank; expanding a plurality of placental stem cells from the Master Cell Bank for a second plurality of population doublings; cryopreserving said placental stem cells to form a Working Cell Bank; expanding a plurality of placental stem cells from the Working Cell Bank for a third plurality of population doublings; and cryopreserving said placental stem cells in individual doses, wherein said individual doses collectively compose a placental stem cell bank. Optionally, a plurality of placental cells from said third plurality of population doublings can be expanded for a fourth plurality of population doublings and cryopreserved in individual doses, wherein said individual doses collectively compose a placental stem cell bank.

In one embodiment, the cells are diluted to about 2 million/ml in 10% human serum albumin (HSA), 10% DMSO in Plasmalyte.

In a preferred embodiment, the donor from which the placenta is obtained (e.g., the mother) is tested for at least one pathogen. If the mother tests positive for a tested pathogen, the entire lot from the placenta is discarded. Such testing can be performed at any time during production of placental cell lots, including before or after establishment of Passage 0 cells, or during expansion culture. Pathogens for which the presence is tested can include, without limitation, hepatitis A, hepatitis B, hepatitis C, hepatitis D, hepatitis E, human immunodeficiency virus (types I and II), cytomegalovirus, herpesvirus, and the like.

### 6. EXAMPLES

### 6.1 Example 1

### Immunomodulation Using Placental Cells

Placental cells, e.g. placental stem cells (PDACs) possess an immunomodulatory effect, including suppression of the proliferation of T cells and natural killer cells. The following experiments demonstrate that placental cells (CD10⁺, CD34⁻, CD105⁺, CD200⁺ placental stem cells) have the ability to modulate the response of T cells to stimulation.

### 6.1.1 PDAC Suppression of T Cell Proliferation Mediated via an IFN-γ Inducible Soluble Mechanism

To address whether PDACs suppress T cell proliferation, and whether such suppression is mediated by a cell to cell contact or soluble factor mediated mechanism, co-culture of BTR (T cells stimulated by anti-CD3, anti-CD28-coated Dynabeads) and either PDACs or PDACs pre-treated with IFN-γ at 500 or 100 units/mL for 24 hours was set up in a coculture allowing cell to cell contact, or in a transwell system at a T cell:PDAC ratio of 10:1.

Bead T-lymphocyte reactions (BTR) were performed by mixing 100,000 T-lymphocytes with anti-CD3 and anti-CD28 coated DynaBeads (Invitrogen) at a bead:T-lymphocyte ratio of 1:3 in a well of a 96-well plate, in the presence or absence of 20,000 PDACs. The mixed cell culture was incubated at 37° C., 5% CO₂, and 90% relative humidity for 6 days. At day 6 all cells were recovered and stained with anti-CD4-PE and anti-CD8 APC (R&D systems, Minneapolis, Minn.).

Suppression of BTR by PDACs was observed under both cell-cell contact and transwell conditions, indicating that suppression was mediated at least partly by a soluble factor. Pre-treatment of PDACs with IFN-γ strongly enhanced PDAC-mediated suppression of BTR. To further confirm suppression of T cell proliferation by PDACs through an IFN-γ inducible mechanism, anti-IFN-γ neutralizing antibody was introduced into a co-culture of PDACs and AbTR (PBMC stimulated by soluble anti-CD3 and anti-CD28) at 2, 8, 16 or 32 μg/ml. Anti-IFN-γ antibody rescued T cell proliferation from PDAC-mediated suppression in a dose dependent manner with ED50 at ˜2 μg/ml. Thus, PDAC immunosuppression of activated T cell proliferation is mediated by IFN-γ.

### 6.1.2 PDACs Induce T_{REG} Cells

The Example demonstrates that placental cells (PDACs) have the ability to induce differentiation of T cells to T_{reg} cells (also known as suppressor T cells), which can downregulate the activity of other T cells.

Naïve CD4⁺ T cells can be induced to differentiate to Th1, Th2, Th17 and regulatory (Treg) phenotypes according to the local cytokine milieu. T_{reg} cells control immunologic tolerance to self-antigens. The skewing of response towards Th17 or Th1 phenotypes, and away from a Treg phenotype, may be responsible for the development and/or progression of certain autoimmune diseases and/or graft-versus-host disease (GVHD).

Induced T_{reg} cells have the phenotype FoxP3⁺ (Forkhead box P3⁺). As such, the effect of PDACs on the expression of FoxP3 in T_{reg} cells was investigated. Peripheral blood cells (PBMC) alone, PBMCs PDACs at a ratio of 1:1000, or PBMCs in the presence of interleukin-2 (IL-2; 300 IU/mL) and IL-15 (125 IU/mL) were cultured for 4 days. A sample of the PBMC were then immunostained for CD25 and FoxP3. CD4⁺ T cells from the PBMC in each condition were isolated using microbeads coated with anti-CD4 antibody. The isolated T cells were treated with 10 μg/mL mitomycin C for 2 hours at 37° C., and tested for their effect on the proliferation of freshly-isolated T cells at a ratio of 1:1, 1:10 and 1:100. PBMC co-cultured with PDACs demonstrated a higher percentage of CD4⁺ cells exhibiting a FoxP3⁺ phenotype, indicating a higher number of T_{reg} cells.

Conclusion: PDACs co-cultured with PBMCs induced T cells to differentiate into cells having a Treg phenotype, and such cells have the ability to suppress T cell proliferation.

### 6.1.3 PDAC Mediated Suppression of T Cell Proliferation is via indoleamine 2,3-dioxygenase (IDO)

This example demonstrates that PDAC T cell immunosuppression is mediated through indoleamine 2,3-dioxygenase (IDO) activity.

As established above, PDACs, when co-cultured with T cells, suppress T cell proliferation. To investigate the mechanism of action of PDAC-mediated suppression of T cell proliferation, pharmacological inhibitors or neutralizing antibodies were used to block the activity of prostaglandin E2 (PGE2), inducible nitric oxide synthase (iNOS), transforming growth factor beta (TGF-β), interleukin-10 (IL-10) and IDO in an in vitro T cell proliferation assay (BTR). No restoration of T cell proliferation was seen by blocking PGE2, iNOS, TGF-beta and IL-10 activity. However, restoration of T cell proliferation was obtained in a dose-dependent manner by adding IDO inhibitor 1 MT (1-methyl tryptophan) at 16, 64, 128 and 256 μM to the T cell proliferation assay.

Tryptophan is required for T cell proliferation. To confirm PDAC-mediated suppression of T cell proliferation in vitro is mediated by IDO, 1 MT at 16, 64, 128 and 256 and an excess of L-trptophan (L-Trp) was introduced into a T cell proliferation assay. 256 μM1 MT and 256 μM L-Trp substantially rescued T cell proliferation from PDAC-mediated suppression in a dose dependent manner.

In another experiment, IDO small interfering RNA (siRNA) or a control siRNA were transfected into PDACs to reduce the expression of IDO. PDACs transfected with IDO siRNA, but not the control siRNA, substantially abolished suppression of T cell proliferation in a BTR assay. These results confirmed PDAC suppression of T cell proliferation is mediated by IDO.

### 6.1.4 The L-System is Required for PDAC Mediated Immunosuppression

The L-system transporter is a heterodimeric membrane transport protein that preferentially transports neutral branched (valine, leucine, isoleucine) and aromatic (tryptophan, tyrosine) amino acids. Because the L-system transporter transports the IDO substrate tryptophan across the plasma membrane, it was hypothesized that the L-system is required for PDAC-mediated immunosuppression. SiRNA specific to the light chain of the L-system (LAT1) transfected into PDACs, and the PDACs were co-cultured with T cells in a proliferation assay. LAT1 specific siRNA, but not a control siRNA, restored T cell proliferation in the presence of PDACs. This result suggests L-system is required for PDAC mediated immunosuppression.

### 6.1.5 Effect of PDACs on T Cell Differentiation and Cytokine Secretion

This example demonstrates that placental stem cells (PDACs) skew T cell differentiation away from Th1 and Th17 subsets and toward the T_{reg} subset.

The ability of PDACs to influence skewing in the T cell compartment was examined by measuring the cytokine secretion in a PMLR including PDACs. Production of IFN-γ, a marker of the Th1 subset, was reduced (≈50%) in T cells cocultured with PDACs in an MLR as compared to T cells in the MLR, alone, or T cells cultured alone. This result was consistent with the suppression of T cell proliferation in the PMLR. In a separate experiment, the presence of PDACs in the PMLR also reduced the percentage of Th17 cells (IL-17-expressing cells) from 10.44% to 4.68% of T cells, and increased the percentage of T_{reg} cells from 8.34% to 12.65%.

To investigate the molecular mechanism of action of PDAC-mediated suppression of Th1 skewing, IDO siRNA was transiently transfected into PDACs by standard techniques. Th1 skewing was carried out as for the BTR reactions (see above) but supplemented with an additional Th1 skewing cytokine cocktail containing IL-2 (200 IU/mL), IL-12 (2 ng/mL) and anti-IL-4 (0.4 μg/ml). The IDO siRNA transfection completely rescued PDAC-mediated suppression of Th1 skewing. As confirmation, the IDO inhibitor 1MT, and tryptophan in excess, also completely rescued Th1 skewing that was suppressed by PDACs.

To determine whether suppression of Th17 skewing by PDACs was mediated through soluble factors, conditioned media from PDACs and PDACs treated with IL-1β were collected on day 1, 2 and 3 of a PDAC culture, and added to a culture of T cells under conditions in which the T cells normally differentiate to the Th17 subset. For Th17 polarization (skewing), 5×10⁵ total T-lymphocytes were stimulated with 5×10⁵ sorted CD14⁴ monocytes, 50 ng/mL anti-CD3 antibody (BD BioScienences), and 100 ng/mL LPS (Sigma Aldrich) in either the presence or absence of 50,000 PDACs for 6 days. The Th17 cell population was analyzed by ICCS staining of IL-17 on CD4 positive population.

PDAC conditioned media suppressed Th17 skewing, and addition of IL-10 treatment enhanced this suppressive effect. Thus, PDAC-mediated suppression of Th17 skewing is mediated by a soluble factor, and IL-10 treatment enhances this effect.

PDAC-mediated induction of IL-10 producing T cell phenotypes is not associated with the skewing of naïve T cell Th1/Th2 lineage commitment. To define the mode of regulation for the observed effects on T cell cytokine secretion in response to PDAC co-culture, a quantitative PCR (qPCR) analysis of IL-10 and TNF mRNA was performed by FACS for CD4 and CD8 T cells sorted from PDAC/BTR co-cultures. A strong reproducible transcriptional induction of IL-10 mRNA in T cells co-cultured with PDACs was observed.

The observed transcriptional regulation of IL-10 indicates an altered effector T cell differentiation pattern in response to PDAC co-culture with the T cells, and could be explained by the specific suppression of the Th1 lineage differentiation, and/or induction of the Th2 lineage differentiation, of naïve T cells. To address this possibility, the PDAC effects on the Th1/Th2 differentiation pattern in the selected naïve CD4 T cells, cultured in the non-polarizing, as well as in Th1- or Th2-polarizing conditions, was monitored. Relative expression levels of T-bet (a transcription factor) and GATA-3 mRNA, respectively the specific transcriptional markers of Th1 and Th2 lineage commitment, were used to quantify the differentiation of T cells isolated from the BTR cultures with or without PDACs. No effects of PDAC co-culture on Th1 or Th2 transcription factors were observed under any of the neutral or lineage specific culture conditions. Therefore, transcriptional induction of the IL-10 producing phenotype by PDACs is not mediated by skewing of Th1/Th2 commitment, consistent with an effect of PDACs on a distinct molecular pathway of regulatory T cell development.

### 6.1.6 PDAC Effect on Macrophage/Monocyte Cytokine Profile

An adherent population of cells obtained from PBMC containing approximately 50% CD14⁺ cells was isolated. A second CD14⁺ population of cells was obtained by positive selection with anti-CD14 coated MACS beads. To investigate the PDACs effect on macrophage and monocyte cytokine secretion profiles, the macrophages/monocytes were treated with LPS for 12 hours and then co-cultured with PDACs for an additional 48 hours. Supernatants were collected and analyzed by a 25-plex Luminex assay for cytokines and growth factors. PDACs, when co-cultured with the PBMC adherent population, suppressed production of IL-1β, IL-8, RANTES and TNF-α, and enhanced production of interleukin-1 receptor agonist (IL-1Ra), by lipopolysaccharide (LPS)-treated PBMC adherent cells. A co-culture of macrophages and PDACs was observed to produce a PDAC cell dose-dependent response in the suppression of TNF-α, as well as the induction of IL-10. The magnitude of the IL-10 response varied more with the macrophage donor.

### 6.1.7 Effect of Placental Stem Cells on Dendritic Cell Maturation and Function

To explore the PDAC-mediated modulation of dendritic cell (DC) maturation and function, monocyte derived immature DC were treated with LPS alone or combination of LPS plus IFN-γ to drive the DC maturation process, in the absence or presence of PDACs. DC maturation was analyzed by FACS staining of the DC maturation markers CD83, CD86 and HLA-DR. DC functional assessment was determined by intracellular staining of IL-12 and by measurement of soluble cytokine production by a Cytometric Bead Assay (BD Pharmingen). PDACs were found to strongly suppress LPS- and LPS+IFN-γ-induced DC maturation, as indicated by down-modulation of CD86, HLA DR and CD83 expression on the DC.

Additionally, PDACs significantly suppressed formation of an LPS+IFN-γ-stimulated IL-12-producing DC population by approximately 50%. Similarly, PDACs suppressed TNF-a production. Unlike previous results in which PDACs increased IL-10 production from T cells, PDACs did not elevate IL-10 production from the dendritic cells.

### 6.1.8 PDACs Suppress LPS Induced IL-23 Production by Monocytes and is Mediated by Soluble Factor

To investigate whether PDACs modulate IL-23 production, 1 million human PBMC were stimulated with LPS at 10 ng/ml for 24 hours in the presence or absence of PDACs at 200 to 100,000 cells/well. IL-23 was determined by ELISA. PDACs strongly suppressed IL-23 production by the PBMC in a dose-dependent manner. Substantially complete suppression (>90%) was observed at PDAC cell numbers above 20,000. Suppression of IL-23 production of approximately 50% was achieved at the lowest PDAC cell doses, 200 cells/well.

In contrast, PDACs did not suppress IL-23 production by LPS-activated monocyte derived dendritic cells. To determine in which PBMC cell type IL-23 production is regulated, CD14 monocyte and CD11c DC fractions were isolated from human PBMC. Each fraction was treated with LPS at 10 ng/ml for 24 hours in either the presence or absence of PDACs in culture. PDACs were observed to specifically down modulate LPS-activated IL-23 production by monocytes, but not by DC.

To understand the mechanism of action of PDAC-mediated down-modulation of PBMC IL-23 production, conditioned medium was collected from cultures of PDACs, PDACs+IFN-γ (100 U/ml), and PDACs+IL-1β (10 ng/ml). The conditioned media were added to cultures of LPS-treated PBMC overnight at different concentrations. PDAC-conditioned medium was observed to strongly suppress IL-23 production by LPS-activated PBMC in a dose dependent manner. Furthermore, conditioned medium from IL-β-treated PDACs showed stronger suppression, in a dose dependent manner, compared to medium conditioned by PDACs alone. In contrast, IFN-γ treated PDACs did not suppress IL-23 production by LPS-activated PBMC. This result suggests that PDAC-mediated suppression of IL-23 production by LPS activated PBMC is mediated by an unidentified soluble factor.

### 6.1.9 PDACs Suppress IL-21 Production in a Th17 Skewing Culture

IL-21 is an important cytokine required for maintenance of Th17 populations. To investigate whether PDACs are able to modulate IL-21 production, PDACs were introduced into a Th17 skewing culture. Soluble IL-21 was measured in the supernatant obtained from the Th-17 skewing culture using the ELISA kit from eBioscience (#88-7216) according to the manufacturer's protocol. PDACs were observed to strongly suppress IL-21 production in the PDAC-Th17 co-culture in comparison to a Th17 skewing culture without PDACs. This result indicates PDAC is able to suppress IL-21 production.

### 6.2 Example 2

### Angiogenesis Using Placental Derived Adherent Cells

### 6.2.1 Secretion of Angiogenic Factors by PDACs

This example demonstrates secretion of angiogenic factors by placental cells (CD10⁺, CD34⁻, CD105⁺, CD200⁺ placental stem cells, also called PDACs).

### 6.2.1.1 Secretome Profiling for Evaluation of Angiogenic Potency of Placental Derived Adherent Cells

MulitplexBead Assay: Placental derived adherent cells at passage 6 were plated at equal cell numbers in growth medium and conditioned media were collected after 48 hours. Simultaneous qualitative analysis of multiple angiogenic cytokines/growth factors in cell-conditioned media was performed using magnetic bead-based multiplex assays (Bio-Plex Pro™, Bio-Rad, Calif.) assays are that allow the measurement of angiogenic biomarkers in diverse matrices including serum, plasma, and cell/tissue culture supernatants. The principle of these 96-well plate-formatted, bead-based assays is similar to a capture sandwich immunoassay. An antibody directed against the desired angiogenesis target is covalently coupled to internally dyed beads. The coupled beads are allowed to react with a sample containing the angiogenesis target. After a series of washes to remove unbound protein, a biotinylated detection antibody specific for a different epitope is added to the reaction. The result is the formation of a sandwich of antibodies around the angiogenesis target. Streptavidin-PE is then added to bind to the biotinylated detection antibodies on the bead surface. In brief, Multiplex assays were performed according to manufacturer's instructions and the amount of the respective angiogenic growth factors in the conditioned media was evaluated.

ELISAs: Quantitative analysis of single angiogenic cytokines/growth factors in cell-conditioned media was performed using commercially available kits from R&D Systems (Minneapolis, Minn.). In brief, ELISA assays were performed according to manufacturer's instructions and the amount of the respective angiogenic growth factors in the conditioned media was evaluated.

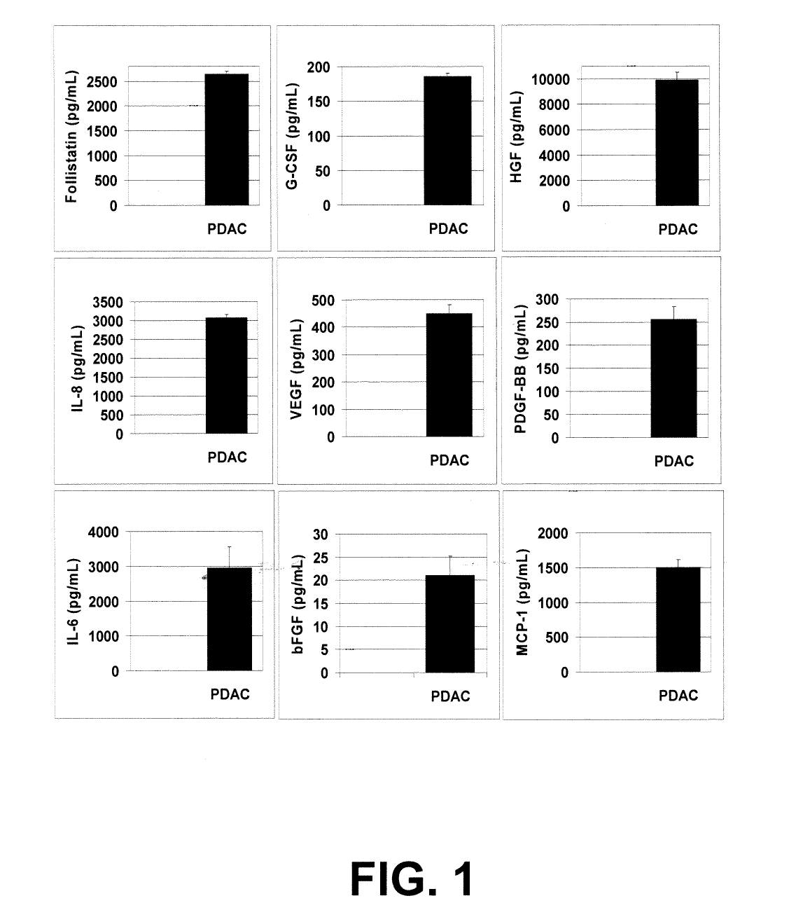

The level of secretion of various angiogenic proteins by PDACs is shown in FIG. 1.

TABLE 1
Multiplex and ELISA results for angiogenic markers

| | | | Secretome |
|---|---|---|---|
| | | | Analysis ELISA, |
| PDAC Marker | Positive | Negative | Multiplex |
| ANG | X | | X |
| EGF | X | | X |
| ENA-78 | X | | X |
| FGF2 | X | | X |
| Follistatin | X | | X |
| G-CSF | X | | X |
| GRO | X | | X |
| HGF | X | | X |
| IL-6 | X | | X |
| IL-8 | X | | X |
| Leptin | X | | X |
| MCP-1 | X | | X |
| MCP-3 | X | | X |
| PDGFB | X | | X |
| PLGF | X | | X |
| Rantes | X | | X |
| TGFB1 | X | | X |
| Thrombopoietin | X | | X |
| TIMP1 | X | | X |
| TIMP2 | X | | X |
| uPAR | X | | X |
| VEGF | X | | X |
| VEGFD | X | | X |

In a separate experiment, PDACs were confirmed to also secrete angiopoietin-1, angiopoietin-2, PECAM-1 (CD31; platelet endothelial cell adhesion molecule), laminin, fibronectin, MMP1, MMP7, MMP9, and MMP10.

### 6.2.2 Functional Characterization of PDACs

This Example demonstrates different characteristics of placental cells (CD10⁺, CD34⁻, CD105⁺, CD200⁺ placental stem cells, also called PDACs) associated with angiogenesis and differentiation capability.

### 6.2.2.1 HUVEC Tube Formation for Evaluation of Angiogenic Potency of PDACs

Human Umbilical Vein Endothelial Cells (HUVEC) were subcultured at passage 3 or less in EGM-2 medium (Cambrex, East Rutherford, N.J.) for 3 days, and harvested at a confluency of approximately 70%-80%. HUVEC were washed once with basal medium/antibiotics (DMEM/F12 (Gibco)) and resuspended in the same medium at the desired concentration. HUVEC were used within 1 hour of preparation. Human placental collagen (HPC) was brought to a concentration of 1.5 mg/mL in 10 mM HCl (pH 2.25), was neutralized with buffer to pH 7.2, and kept on ice until used. The HPC was combined with the HUVEC suspension at a final cell concentration of 4000 cells/μl. The resulting HUVEC/HPC suspension was immediately pipetted into 96-well plates at 3 μl per well (plate perimeter must be pre-filled with sterile PBS to avoid evaporation, n=5 per condition). HUVEC drops were incubated at 37° C. and 5% CO₂ for 75-90 minutes without medium addition to allow for collagen polymerization. Upon completion of “dry” incubation, each well was gently filled with 200 μl of conditioned PDAC medium (n=2 cell lines) or control medium (e.g., DMEM/F12 as the negative control, and EGM-2 as the positive control) and incubated at 37° C. and 5% CO₂ for 20 hrs. Conditioned medium was prepared by incubating PDACs at passage 6 in growth medium for 4-6 hours; after attachment and spreading, the medium was changed to DMEM/F12 for 24 hours. After incubation, the medium was removed from the wells without disturbing the HUVEC drops and the wells were washed once with PBS. The HUVEC drops were then fixed for 10 seconds and stained for 1 minute using a Diff-Quik cell staining kit and subsequently rinsed 3× times with sterile water. The stained drops were allowed to air dry and images of each well were acquired using the Zeiss SteReo Discovery V8 microscope. The images were then analyzed using the computer software package ImageJ and/or MatLab. Images were converted from color to 8-bit grayscale images and thresholded to convert to a black and white image. The image was then analyzed using the particle analysis features, which provided pixel density data, including count (number of individual particles), total area, average size (of individual particles), and area fraction, which equates to the amount endothelial tube formation in the assay.

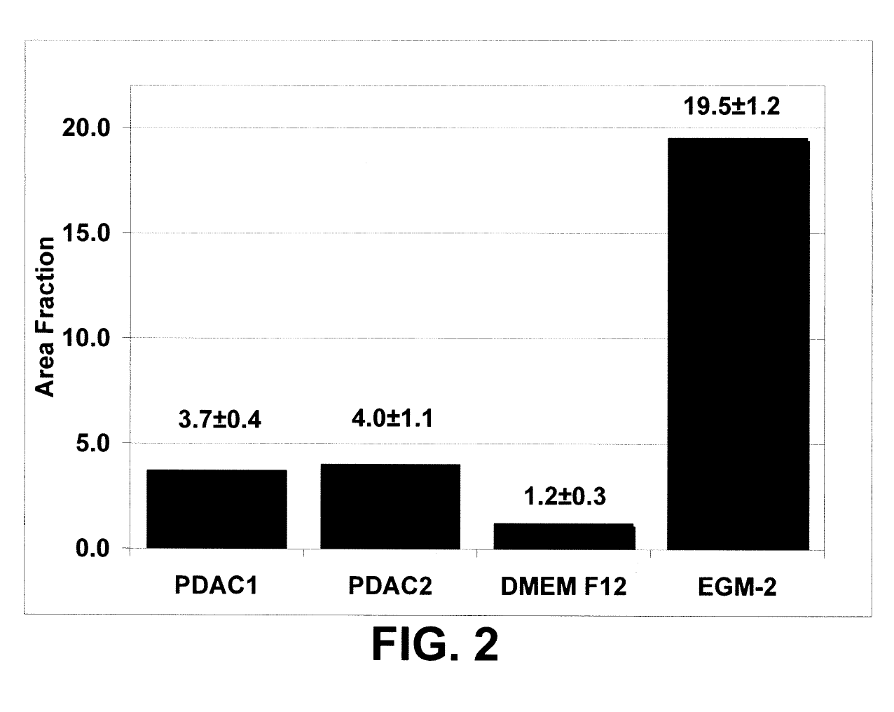

The conditioned medium exerted an angiogenic effect on endothelial cells, as demonstrated by the induction of tube formation (see FIG. 2).

### 6.2.2.2 HUVEC Migration Assay

This experiment demonstrated the angiogenic capacity of placental derived adherent cells. HUVECs were grown to monolayer confluence in a fibronectin (FN)-coated 12-well plate and the monolayer was “wounded” with a 1 mL plastic pipette tip to create an acellular line across the well. HUVEC migration was tested by incubating the “wounded” cells with serum-free conditioned medium (EBM2; Cambrex) obtained from PDACs after 3 days of growth. EBM2 medium without cells was used as the control. After 15 hours, the cell migration into the acellular area was recorded (n=3) using an inverted microscope. The pictures were then analyzed using the computer software package ImageJ and/or MatLab. Images were converted from color to 8-bit grayscale images and thresholded to convert to a black and white image. The image was then analyzed using the particle analysis features, which provided pixel density data, including count (number of individual particles), total area, average size (of individual particles), and area fraction, which equates to the amount endothelial migration in the assay. The degree of cell migration was scored against the size of the initially recorded wound line and the results were normalized to 1×10⁶ cells.

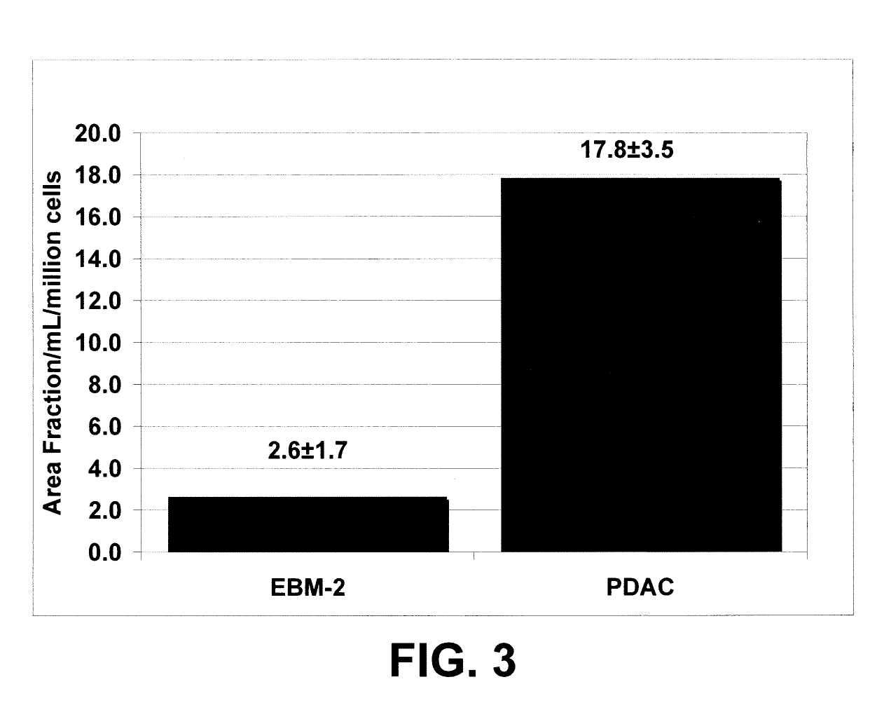

The trophic factors secreted by placental derived adherent cells exerted angiogenic effects on endothelial cells, as demonstrated by the induction of cell migration (FIG. 3).

In a separate experiment, HUVECs were cultured in the bottom of 24 well-plates for overnight establishment in EGM2, followed by a half-day starvation in EBM. Concurrently, media-cultured PDACs were thawed and cultured in transwells (8 μM) overnight. After the EC starvation, the conditioned serum-free DMEM, along with the transwell, was transferred over to the ECs for overnight proliferation. 4 replicates were included in each experiment, and proliferation after 24 hrs was assessed with Promega's Cell Titer Glo Assay. EBM-2 medium was used as the negative control, and EGM-2 was used as the positive control. Error bars denote standard deviations of analytical replicates (n=3).

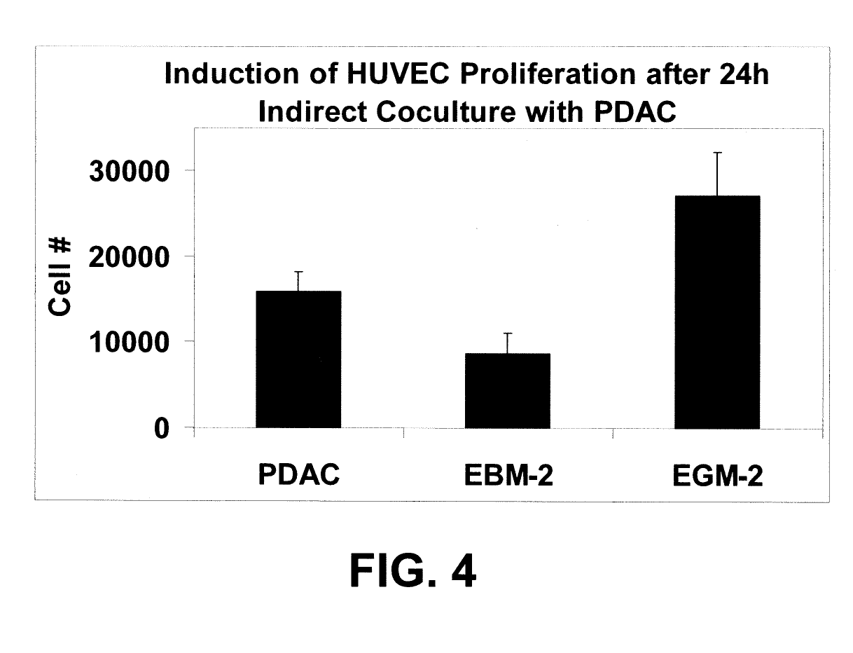

The trophic factors secreted by PDACs resulted in an increase in HUVEC cell number, which is indicative of HUVEC proliferation. See FIG. 4.

### 6.2.2.3 Tube Formation for Evaluation of Angiogenic Potency of Placental Derived Adherent Cells

PDACs were grown either in growth medium without VEGF or EGM2-MV with VEGF to evaluate the angiogenic potency of the cells in general, as well as the effect of VEGF on the differentiation potential of the cells. HUVECs, as control cells for tube formation, were grown in EGM2-MV. The cells were cultured in the respective media for 4 to 7 days until they reached 70-80% confluence. Cold (4° C.) MATRIGEL™ solution (50 μL; BD Biosciences) was dispensed into wells of a 12-well plate and the plate was incubated for 60 min at 37° C. to allow the solution to gel. The PDAC and HUVEC cells were trypsinized, resuspended in the appropriate media (with and without VEGF) and 100 μl of diluted cells (1 to 3×10⁴ cells) were added to each of the MATRIGEL™-containing wells. The cells on the polymerized MATRIGEL™, in the presence or absence of 0.5 to 100 ng VEGF, were placed for 4 to 24 hours in a 5% CO₂ incubator at 37° C. After incubation the cells were evaluated for signs of tube formation using standard light microscopy.

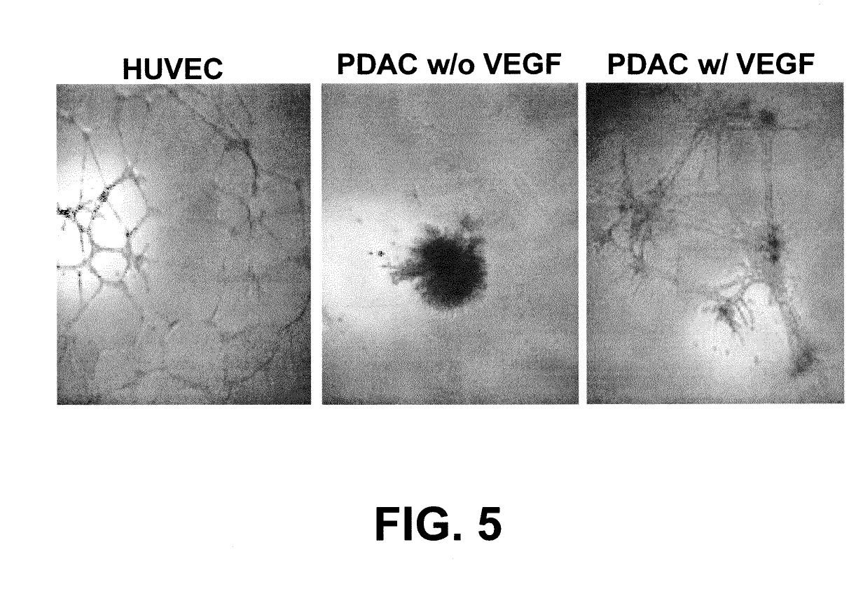

PDACs displayed minimal tube formation in the absence of VEGF, but were induced/differentiated to form tube-like structures through stimulation with VEGF. See FIG. 5.

### 6.2.2.4 Hypoxia Responsiveness for Evaluation of Angiogenic Potency of Placental Derived Adherent Cells

To evaluate the angiogenic functionality of endothelial cells and/or endothelial progenitors, cells can be assessed in regard to their capability to secrete angiogenic growth factors under hypoxic and normoxic conditions. Culture under hypoxic conditions usually induces an increased secretion of angiogenic growth factors by either endothelial cells or endothelial progenitor cells, which can be measured in the conditioned media. Placental derived adherent cells were plated at equal cell numbers in their standard growth medium and grown to approximately 70-80% confluence. Subsequently, the cells were switched to serum-free medium (EBM-2) and incubated under normoxic (21% O₂) or hypoxic (1% O₂) conditions for 24 h. The conditioned media were collected and the secretion of angiogenic growth factors was analyzed using commercially available ELISA kits from R&D Systems. The ELISA assays were performed according to manufacturer's instructions and the amount of the respective angiogenic growth factors (VEGF and IL-8) in the conditioned media was normalized to 1×10⁶ cells.

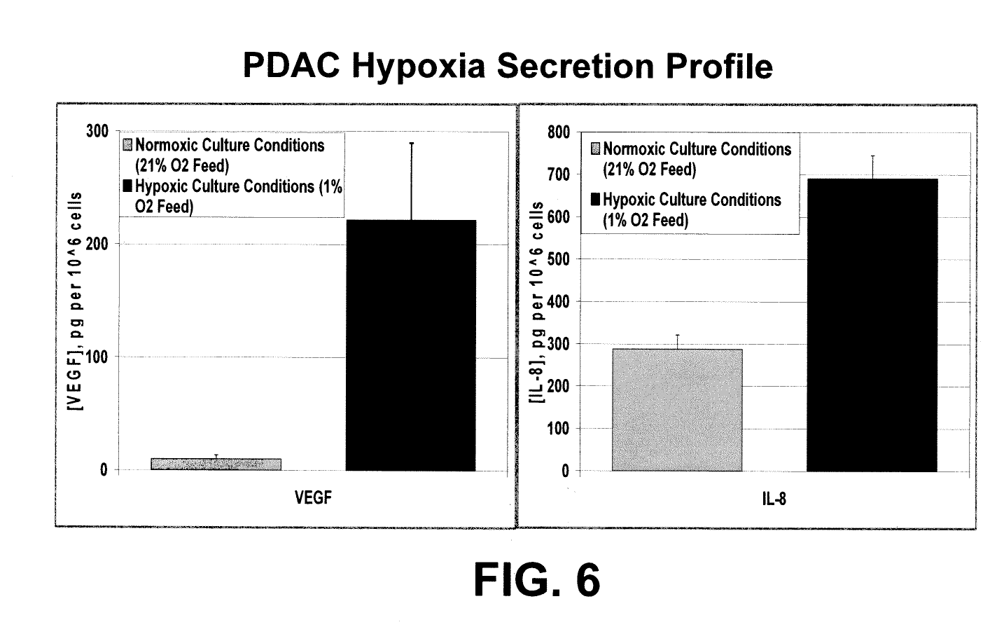

Placental derived adherent cells displayed elevated secretion of various angiogenic growth factors under hypoxic conditions. See FIG. 6.

### 6.2.2.5 HUVEC Response to PDAC-Conditioned Medium

PDACs were cultured for 48 hours in growth medium containing 60% DMEM-LG (Gibco); 40% MCBD-201 (Sigma); 2% FBS (Hyclone Labs), 1× insulin-transferrin-selenium (ITS); 10 ng/mL linoleic acid-bovine serum albumin (LA-BSA); 1 n-dexamethasone (Sigma); 100 μM ascorbic acid 2-phosphate (Sigma); 10 ng/mL epidermal growth factor (R & D Systems); and 10 ng/mL platelet-derived growth factor (PDGF-BB) (R & D Systems), and then cultured for an additional 48 hrs in serum-free media. Conditioned medium from PDAC culture was collected and used to stimulate serum-starved HUVECs for 5, 15, and 30 minutes. The HUVECs were subsequently lysed and stained with a BD™ CBA (Cytometric Bead Assay) Cell Signaling Flex Kit (BD Biosciences) for phosphoproteins known to play a role in angiogenic pathway signaling. PDACs were found to be strong activators of AKT-1 (which inhibits apoptotic processes), AKT-2 (which is an important signaling protein in the insulin signaling pathway, and ERK 1/2 cell proliferation pathways in HUVECs. These results further demonstrate the angiogenic capability of PDACs.

### 6.2.3 Induction of Angiogenesis by PDACs

This Example demonstrates that PDACs, as described in Example 2, above, promote angiogenesis in an in vivo assay using chick chorioallantoic membrane (CAM).

Two separate CAM assays were conducted. In the first CAM assay, intact cell pellets from different preparations of PDAC were evaluated. In the second CAM assay, supernatants of different PDAC preparations were evaluated. Fibroblast growth factor (bFGF) was used as a positive control, and MDA-MB-231 human breast cancer cells as a reference, vehicle and medium controls were used as negative controls. The endpoint of the study was to determine the blood vessel densities of all treatment and control groups.

### 6.2.3.1 CAM Assay Using PDACs

PDACs, prepared as described above and cryopreserved, were used. PDACs were thawed for dosing and the number of cells dosed on the CAM was determined.

Study Design: The study included 5 groups with 10 embryos in each group. The design of the study is described in Table 2.

TABLE 2
Study groups, chick chorioallantoic membrane angiogenesis assay.

| Group | # of | | |
|---|---|---|---|
| No. | Embryos | Treatment | End Point |
| 1 | 10 | Vehicle control (40 | Blood vessel |
| | | μl of PBS/MATRIGEL ™ | density score |
| | | mixture, 1:1 by volume) | |
| 2 | 10 | Positive control, treated | Same as group 1 |
| | | with bFGF (100 ng/CAM in | |
| | | 40 μl of DMEM/MATRIGEL ™ | |
| | | mixture, 1:1) | |
| 3 | 10 | Medium control (40 μl of | Same as group 1 |
| | | DMEM) | |
| 4 | 10 | PDAC | Same as group 1 |
| 5 | 10 | MDA-MB-231 cells P34, | Same as group 1 |
| | | Lot No. 092608 | |

CAM Assay Procedure: Fresh fertile eggs were incubated for 3 days in a standard egg incubator at 37° C. for 3 days. On Day 3, eggs were cracked under sterile conditions and embryos were placed into twenty 100 mm plastic plates and cultivated at 37° C. in an embryo incubator with a water reservoir on the bottom shelf. Air was continuously bubbled into the water reservoir using a small pump so that the humidity in the incubator was kept constant. On Day 6, a sterile silicon “O” ring was placed on each CAM, and then PDAC at a density of 7.69×10⁵ cells/40 μL of medium/MATRIGEL™ mixture (1:1) were delivered into each “O” ring in a sterile hood. Tables 2A and 2B represent the number of cells used and the amount of medium added to each cell preparation for dosing. Vehicle control embryos received 40 μL of vehicle (PBS/MATRIGEL™, 1:1), positive controls received 100 ng/ml bFGF in 40 μl of DMEM medium/MATRIGEL™ mixture (1:1), and medium controls received 40 μl of DMEM medium alone. Embryos were returned to the incubator after each dosing was completed. On Day 8, embryos were removed from the incubator and kept at room temperature while blood vessel density was determined under each “O” ring using an image capturing system at a magnification of 100×.

Blood vessel density was measured by an angiogenesis scoring system that used arithmetic numbers 0 to 5, or exponential numbers 1 to 32, to indicate the number of blood vessels present at the treatment sites on the CAM. Higher scoring numbers represented higher vessel density, while 0 represented no angiogenesis. The percent of inhibition at each dosing site was calculated using the score recorded for that site divided by the mean score obtained from control samples for each individual experiment. The percent of inhibition for each dose of a given compound was calculated by pooling all results obtained for that dose from 8-10 embryos.

TABLE 3
Amount of medium added to each cell preparation for
normalization of the final cell suspension for dosing

| | Pellet | Normalization with DMEM | Final Volume of |
|---|---|---|---|
| Cell Line | size | and MATRIGEL ™ | Cell Suspension |
| PDAC | 260 μL | 0 μL + 260 μL | 520 μL |
| | | MATRIGEL ™ | |
| MDA-MB-231 | 40 μL | 220 μL + 260 μL | 520 μL |
| | | MATRIGEL ™ | |

PDACs were used at Passage 6.

### Results

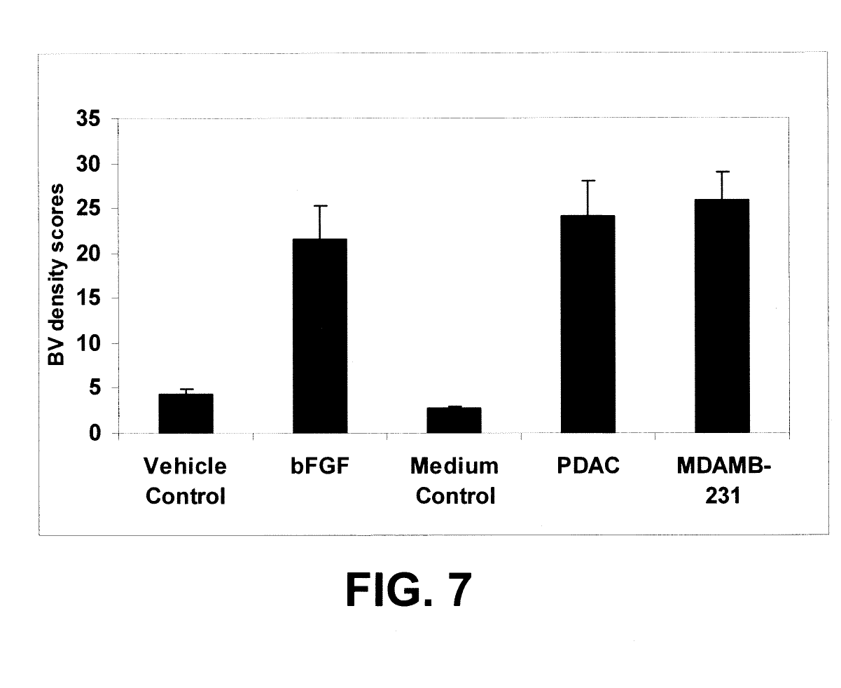

The results of blood vessel density scores are presented in FIG. 7. The results clearly indicate that the blood vessel density scores of chick chorioallantoic membranes treated with PDAC cell suspensions, or 100 ng/mL of bFGF, or MDAMB231 breast cancer cell suspensions were statistically significantly higher compared to those of the vehicle control CAMs (P<0.001, Student's “t” test). The medium used for culturing PDACs (negative control) did not have any effect on the blood vessel density. Further, the induction of blood vessel density of PDAC preparations showed some variation, but the variations were not statistically significant.

### 6.2.3.2 CAM Assay Using PDAC Supernatants

Supernatant samples from MDA-MB-231 cells and PDACs were used in a second CAM assay as described above. bFGF and MDA-MB-231 supernatants were used as positive controls, medium and vehicle were used as negative controls.

Study Design: The study included 5 groups with 10 embryos in each group. The design of the study is described in Table 4.

TABLE 4
Study Design - CAM assay using cell supernatants

| Group | # of | | |
|---|---|---|---|
| No. | Embryos | Treatment | End Point |
| 1 | 10 | Vehicle control (40 | Blood vessel |
| | | μl of PBS/MATRIGEL ™ | density score |
| | | mixture, 1:1 by volume) | |
| 2 | 10 | Positive control, treated | Same as group 1 |
| | | with bFGF (100 ng/CAM in | |
| | | 40 μl of DMEM/MATRIGEL ™ | |
| | | mixture, 1:1) | |
| 3 | 10 | Medium control (40 μl of | Same as group 1 |
| | | DMEM) | |
| 4 | 10 | Supernatant of PDACs | Same as group 1 |
| 5 | 10 | Supernatant of | Same as group 1 |
| | | MDAMB231 cells | |
| | | (P34) | |

PDAC supernatants were obtained from cells at Passage 6.

CAM Assay Procedure: The assay procedure was the same as described in section 6.3.1, above. The only difference was that supernatant from each stem cell preparation or from MDA-MB-231 cells was used as test material. For dosing, each supernatant was mixed with MATRIGEL™ (1:1 by volume) and 404 of the mixture was dosed to each embryo.

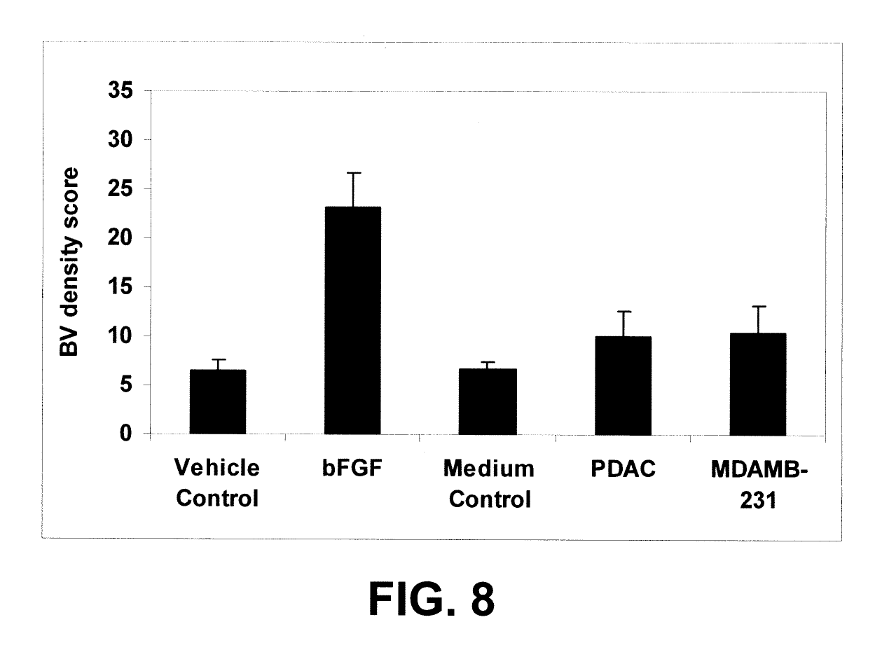

Results: Blood vessel density scores (see FIG. 8) indicate that the induction of blood vessel formation by the supernatant of each stem cell preparation differed. Supernatant samples from PDACs showed significant effect on blood vessel induction with P<0.01, P<0.001, and P<0.02 (Student's “t” test) respectively. As expected, positive control bFGF also showed potent induction of blood vessel formation as seen above in CAM assay no. 1 (P<0.001, Student's “t” test). However, supernatant from MDA-MB-231 human breast cancer cells did not show significant induction on blood vessel formation compared to the vehicle controls. As previously shown, culture medium alone did not have any effect.

### 6.3 Example 3

### PDACs Exhibit Neuroprotective Effects

This Example demonstrates that PDACs have a neuroprotective effect in low-oxygen and low-glucose conditions using an oxygen-glucose deprivation (OGD) insult assay, and a reactive oxygen species assay. In addition, PDACs express neuroprotective moieties, including the neurotrophic factors BDNF, GDNF, NT-3, NT-4/5, and antioxidative enzymes hemoxygenase-1, catalase, superoxide dismutase-1 and aldehyde oxidase-1, and the expression and secretion of some of these moieties are elevated after hypoxic insult. As such, these results indicate that PDACs would be useful in treating CNS injuries, e.g., an SCI or TBI, typically characterized by neuronal loss of function or degeneration by necrosis, apoptosis, demyelination, and other forms of loss of function.

Human neurons (ScienCell, catalog #1520) were cultured as per manufacturer's recommendations. Briefly, culture vessels were coated with Poly-L-Lysine (2 μg/mL) in sterile distilled water for 1 hour at 37° C. The vessel was washed with double distilled H₂O three times. Neuron Medium (ScienCell) was added to vessel and equilibrated to 37° C. in an incubator. Neurons were thawed, and added directly into the vessels without centrifugation. During subsequent culture, medium was changed the day following culture initiation, and every other day thereafter. The neurons were typically ready for insult by day 4.

OGD medium (Dulbecco's Modified Eagle's Medium-Glucose Free) was prepared by first warming the medium in a water bath, in part to reduce the solubility of oxygen in the liquid medium. 100% nitrogen was bubbled for 30 minutes through the medium using a 0.5 μm diffusing stone to remove dissolved oxygen. HEPES buffer was added to a final concentration of 1 mM. Medium was added directly to the neurons at the end of the sparge. A small sample of the medium was aliquoted for confirmation of oxygen levels using a dip-type oxygen sensor. Oxygen levels were typically reduced to 0.9% to about 5.0% oxygen.

A hypoxia chamber was prepared by placing the chamber in an incubator at 37° C. for at least 4 hours (overnight preferred) prior to gassing. Medium in the culture vessels was removed and replaced with de-gassed medium, and the culture vessels were placed in the hypoxia chamber. The hypoxia chamber was then flushed with 95% N₂/5% CO₂ gas through the system at a rate of 20-25 Lpm for at least 5 minutes. The system was incubated in the incubator at 37° C. for 4 hours, with degassing of the chamber once more after 1 hour.

At the conclusion of the insult procedure, OGD medium was aspirated and warm medium was added to the neurons. 24-28 hours later, PDACs and neurons were plated at equal numbers at 100,000 cells each per well of a 6-well plate suspended in Neuronal Medium were added to the neurons and co-cultured for 6 days.

Photomicrographs were taken of random fields in a 6-well plate for each condition. Cells having a typical neuron morphology were identified, and neurite lengths were recorded. The average length of the neurites positively correlated to neuronal health, and were longer in co-cultures of neurons and PDACs, indicating that the PDACs were protecting the cells from the insult.

### Reactive Oxygen Species Assay

The ability of PDACs to scavenge reactive oxygen species, and to protect cells from such species, was determined in an assay using hydrogen peroxide as a reactive oxygen species generator.

Assay Description: Target cells (Astrocytes, ScienCell Research Laboratories) were seeded in 96-well black well plates pre-coated with poly-L-lysine at 6000/cm². The astrocytes are allowed to attach overnight in growth medium at 37° C. with 5% carbon dioxide. The following day, the culture media was removed and the cells were incubated with cell permeable dye DCFH-DA (Dichlorofluorescin diacetate), which is a fluorogenic probe. Excess dye was removed by washing with Dulbecco's Phosphate Buffered Saline or Hank's Buffered Salt Solution. The cells were then insulted with reactive oxygen species by addition of 1000 μM hydrogen peroxide for 30-60 minutes. The hydrogen peroxide-containing medium was then removed, and replaced with serum-free, glucose-free growth medium. PDACs were added at 6000/cm², and the cells were cultured for another 24 hours. The cells were then read in a standard fluorescence plate reader at 480Ex and 530Em. The reactive oxygen species content of the medium was directly proportional to the levels of DCFH-DA in the cell cytosol. The reactive oxygen species content was measured by comparison to pre-determined DCF standard curve. All experiments were done with N=24.

For the assay, 1× DCFH-DA was prepared immediately prior to use by diluting a 20× DCFH-DA stock solution to 1× in cell culture media without fetal bovine serum, and stirring to homogeneity. Hydrogen Peroxide (H₂O₂) dilutions were prepared in DMEM or DPBS as necessary. A standard curve was prepared as a 1:10 dilution series in concentration range 0 μM to 10 μM by diluting 1 mM DCF standard in cell culture media, transferring 100 μl of DCF standard to a 96 well plate suitable for fluorescent measurement, and adding 100 μl of cell lyses buffer. Fluorescence was read at 480Ex and 530Em.

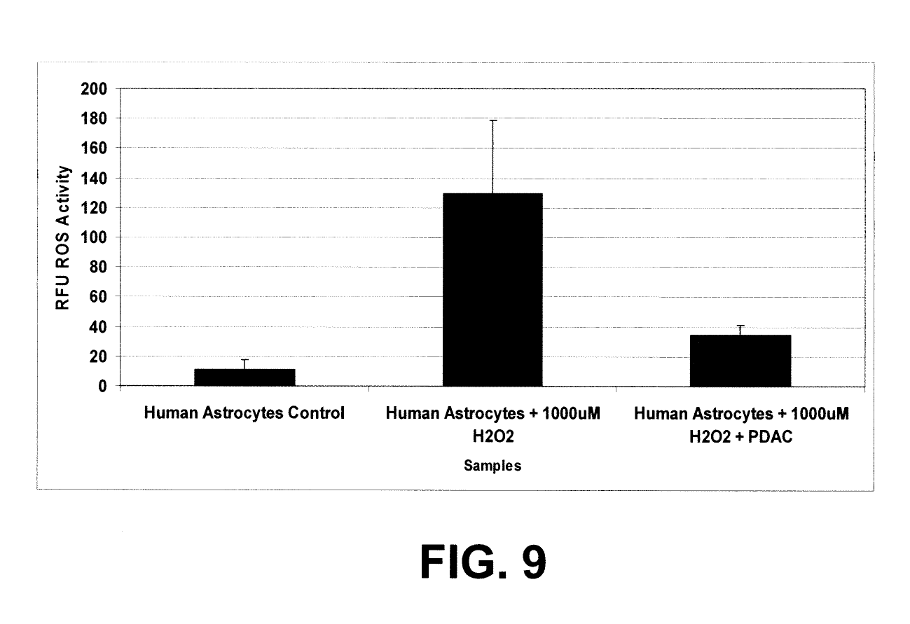

Results: PDACs significantly reduced the concentration of reactive oxygen species in the astrocyte co-cultures. See FIG. 9.

### Expression and Secretion of Neuroprotective Moieties

To evaluate the gene expression of neuroprotective moieties under normal (normoxic) and injury/disease-relevant (hypoxic) conditions, PDACs were seeded in six-well tissue culture dishes at 6000 cells/cm2 and allowed to grow in media for 24 hours. Subsequently, the growth media was removed and the cells were washed with PBS. Serum-free DMEM media was then added to the cells and the cultures were incubated for three hours at 37° C., 5% CO₂, 21% O₂, 90% humidity. For the assessment of PDACs gene expression under hypoxic conditions, serum free media that was equilibrated to 1% O₂ was added to the cells and the cultures were placed for three hours in a hypoxia chamber at 37° C., 5% CO₂, 1% O₂, 90% humidity. After incubation in said conditions, total RNA was extracted from the cells using a RNeasy Mini kit (Qiagen, Valencia, Calif.) to the manufacturer's instructions. Quantification of gene transcripts was conducted using quantitative Real Time Polymerase Chain Reaction (qRT-PCR) techniques. Secretion of BDNF, GDNF, NT-3, NT-4/5 into the supernatant was determined using solid phase sandwich ELISA Immunoassay systems (R&D Systems, Minneapolis, Wis.), according to manufacturers instructions. Protein concentrations were determined colorimetrically by correlation of absorbance or the reporter chromagen (tetramethylbenzidine) at 450 nM with known respective standards.

To evaluate the secretion of neuroprotective moieties, PDACs were seeded in six-well tissue culture dishes at 6000 cells/cm2 and allowed to grow in PDACs media for 24 hours. Subsequently, the growth media was removed and the cells were washed with PBS. Serum-free DMEM media was then added to the cells and the cultures were incubated for an additional 3 hours at 37° C., 5% CO₂, 21% O₂, 90% humidity in a standard tissue culture incubator for the assessment of PDACs secretion of trophic factors under normoxic conditions. For the assessment of PDAC secretion under hypoxic conditions, serum free media that was equilibrated to 1% O₂ was added to the cells and the cultures were placed for three hours in a hypoxia chamber at 37° C., 5% CO₂, 1% O₂, 90% humidity.

Upon completion of the incubation period, cell-conditioned medium was collected from the tissue culture vessels, frozen at −80° C., and subsequently analyzed as described below.

Results: The expression levels of antioxidative enzyme transcripts expressed by PDACs under normoxic conditions were compared to that of human astrocytes. The measured Ct values indicated higher expression of the antioxidative enzymes Catalase (CAT), Hemoxygenase-1 (HMOX-1), Aldehyde oxidase-1 (AOX-1) and Superoxide dismutase-1 (SOD-1) by PDACs, suggesting superior neutralization of ROS in comparison to cultured astrocytes. In addition, the expression levels of neurotrophic factors expressed by PDACs under normoxic conditions were compared to human astrocyte controls. The measured Ct values indicate expression of the known neurotrophic factors Brain-derived Neurotrophic Factor (BDNF), Glial-derived Neurotrophic Factor (GDNF), Neurotrophin-3 (NT-3), Neurotrophin-4/5 (NT-4/5) by PDACs, suggesting that PDACs could exhibit neuroprotective functionality via a number of factors. To evaluate the inducibility of these genes of interest under injury/disease-relevant conditions, PDACs were cultured for 3 hours under hypoxic conditions prior to assessment of gene expression. The expression of BDNF, GDNF, NT-3 and NT-4/5 by PDACs were elevated 2.5-fold, 5-fold, 30-fold, and 15-fold respectively after hypoxic culture. These results suggest that PDACs can respond appropriately in a disease-relevant environment via the regulation of neuroprotective factors, further supporting the hypothesis that PDACs possess neuroprotective functionality.

To determine whether the elevated transcripts resulted in increased protein expression, conditioned supernatants were assessed for the presence of neurotrophic factors. The presence of BDNF, GDNF, NT-3 and NT-4 neurotrophic factors were confirmed at substantial levels after 3 hrs of culture in the conditioned media and a 78% and 100% increase of BDNF and NT-3 secretion, respectively after 3 hrs of hypoxic insult, corroborating the observed regulation on the gene expression level. These results support the notion that PDACs will therapeutically modulate a variety of pathological processes associated with acute CNS injury via the expression of antioxidative enzymes and neurotrophic factors, for example the hypoxia-driven excessive accumulation of ROS and injury of neuronal cells.

### 6.4 Example 4

### Treatment of SCI Using PDACs in a Rat SCI Model

This Example provides an exemplary model and method for evaluating the effects of CD10⁺, CD34⁻, CD105⁺, CD200⁺ placental stem cells, also called PDACs, on an SCI, and in particular, for evaluating the immune rejection, migration, and differentiation, of PDACs transplanted to the uninjured and injured spinal cord of rats. The model provides for the assessment of the effects of PDACs administration alone or in combination with secondary treatment options, e.g., co-administration with methylprednisolone, lithium, and/or cyclosporin A. The effects of PDACs on function, including recovery of locomotary activity (BBB scores), regeneration of corticospinal tract and serotonergic axons, and white matter area in the spinal cord, are assessed at 12 weeks after injury, with and without cyclosporin, compared to control rats without cell transplants. The cells are transplanted into the spinal cord shortly, 2 weeks, and 6 weeks after injury, to simulate transplantation of cells into the acute, subacute, and chronic phase of SCI. The survival, migration, and differentiation of PDACs administered at 0, 1, 2, 3, 4, and 6 weeks after injury are assessed. In addition, expression of neurogenic growth factors, e.g., neurotrophins, following the administration of PDACs can be assessed utilizing gene chip, RT-PCR and ELISA methodology.

Experimental Design

In vivo persistence of PDACs. PDACs are injected into the central gray region at the upper edge of T9 and lower edge of T10 vertebral segment of the rat spinal cord at 0, 1, 2, 3, 4, and 6 weeks with or without infliction of SCI with a 25 mm weight drop (n=4/group). After 6 weeks, rats are anesthetized with 60 mg/kg pentobarbital, perfused with formaldehyde, and the spinal cords are sectioned horizontally and examined with an epifluorescent dissecting microscope. The distribution of PDACs at various distances from the injections sites are measured via fluorescence, and sections are stained immunohistologically for beta-3-tubulin (neuron), GFAP (astrocyte), nestin (progenitor) markers.

Treatments. Rats administered with PDACs are treated with methylprednisolone (MP, 30 mg/kg bolus at the time of transplant), lithium (Li, 100 mg/kg/day for 6 weeks), and cyclosporin (CsA, 10 mg/kg/day) and the number, distribution, and characteristics of the transplanted PDACs at 6 weeks after injury and transplantation are assessed. The effects of PDACs alone, MP alone, Li alone, CsA alone, or MP+Li are assessed. To quantify the cells, the amounts of human DNA and green fluorescent protein (GFP) in the spinal cord are measured. Short-medium-term GFP expression in PDACs is achieved by Amaxa-based electroporatation of a plasmid vector encoding a constitutive GFP expression cassette. Longer-term expression is achieved by the use of a lentiviral vector encoding constitutive GFP expression.

Gene/Protein Expression. RT/PCR and ELISA is used to measure mRNA and protein levels of LIF, BDNF, GDNF, NT3, NGFA, and GFP in animals that are not treated or treated with PDACs alone, PDACs plus MP, PDACs plus MP and L1, and PDACs plus MP, L1 and CsA.

Recovery/Regeneration. PDACs are transplanted 2 weeks and 6 weeks after injury with or without CsA, and the animals are kept for 12 weeks. Locomotor recovery (BBB) is assessed and histological studies are performed.

Protocol

Anesthesia. Sprague-Dawley rats which are 77±1 day old are subjected to laminectomy. The rats are anesthetized with intraperitoneal pentobarbital (45 mg/kg female, 65 mg/kg male). Rats that do not become deeply anesthetized within five minutes are excluded from the experiment. For delayed transplants of cells into spinal cord at 1 week and 4 weeks after injury, the rats are anesthetized by spontaneous respiration of isoflurane via a head-cone (5% induction for 5 minutes and then 1% maintenance).

Spinal Cord Injury. After shaving the rats and preparing the surgery site with betadine, a midline dorsal incision is made to expose the T8-11 vertebral column and a T9-10 laminectomy is carried out to expose the underlying T13 spinal cord. The rats are suspended with clamps placed on the TS and T11 dorsal processes. At one hour after induction of anesthesia, a 10-gram rod is dropped 25 mm onto T13 spinal cord. A thin (100μ) sheet of polylactic acid and polycaprilactone is placed over the dura to prevent adhesions, and a piece of autologous subcutaneous fat is placed on the laminectomy site to retard scar formation. Muscle is sutured at the midline with silk above and below the laminectomy. Skin is closed with stainless steel clips. The clips are removed a week later.

Cell Transplantation. The dura is incised with a 26-gauge tuberculin syringe and a 1-microliter suspension of 200,000 cells is injected into the spinal cord. For delayed transplantation, the laminectomy site is reopened after anesthesia with isoflurane, a small dural incision is made, and a micropipette is used to inject two 1-microliter suspensions of 200,000 cells into the spinal cord rostral and caudal to the impact site.

Postoperative care. The rats are maintained on heating pads until they wake up. Rats showing cyanosis (from the color of their feet) receive transoral tracheal suction to clear secretions and stimulate respiration. Atropine at 0.04 mg/kg IM or glycopyrolate at 0.5 mg/kg IM is optionally administered to reduce intraoperative secretion build up if there are more incidents of respiratory obstruction. Rats showing signs of dehydration (e.g., the skin of the back is pinched and does not settle down in a second) receive 5-10 ml subcutaneous saline injection (5 ml female, 10 ml male). All rats receive 50 mg/kg of cefazolin subcutaneously daily for 7 days, to reduce urinary tract and wound infections.

Postoperative analgesia. Spinal cord injured rats generally do not show evidence of pain because the injury causes anesthesia at and below the injury site. However, for animals subjected to laminectomy only, i.e., without spinal cord injury, and showing postoperative pain, a local anesthetic, Bupivacaine (Marcaine) is administered at the surgical site at a maximum dose of 2 mg/kg body weight. Each animal is monitored for evidence of pain and additional pain relief is provided as needed.

Long-term care. Rats are inspected daily and assessed weekly for locomotor scores (BBB). First, the animals are inspected twice daily and manually expressed if palpation indicates >1 ml urine in their bladders. Rats with cloudy and bloody urine, indicative of bladder infection, after initial 7 day period receive 2.5 mg/kg/day of Baytril (a fluoroquinolone antibiotic) for 7-10 days. If this does not clear up the infection, the rats are euthanized. Second, the rats are kept on sterile white paper litter (Alpha Dry), which keeps the rats dry and shows presence of hemorrhagic urine. Rats with hemorrhagic urine are set aside and cared for in isolation from other rats, to avoid transferring infections. Third, if the rats show evidence of pain (vocalization, sensitivity to touch) or autophagia (biting of the dermatomes below the injury site manifested by hair loss or skin penetration), the rats are given daily oral acetaminophen (64 mg/kg/day “Baby Tylenol” orally) until their skin lesions are completely healed. If no correctable causes of the pain are found, the rats are euthanized. The animals are weighed daily for the first week and weekly thereafter.

Euthanasia. All animals will be deeply anesthetized with pentobarbital (100 mg/kg female-male doses) and decapitated for molecular studies or perfused with 4% paraformaldehyde solutions for fixation and histology study.

### 6.5 Example 5

### Treatment of TBI Using PDACs in a Rat TBI Model

This Example provides an exemplary model and method for evaluating the effects of CD10⁺, CD34⁻, CD105⁺, CD200⁺ placental stem cells, also called PDACs, on a TBI. Without intending to be bound to any particular theory or mechanism of action, it is believed that TBI results in a decrease in splenic mass that correlates with an increase in circulating immune cells leading to increased blood brain barrier permeability. Thus, this method provides for the assessment of the ability of PDACs to modulate immunologic response; to co-localize with splenocytes to promote splenocyte proliferation and secretion of anti-inflammatory cytokines such as IL-4 and IL-10; preserve splenic mass; and to maintain the integrity of the blood brain barrier following induced TBI.

In Vivo Methods

Controlled cortical impact injury. A controlled cortical impact (CCI) device, for example, eCCI Model 6.3; VCU, Richmond, Va. is used to administer a unilateral brain injury as described by Lighthall J., Neurotrauma 5, 1-15 (1988)), the disclosure of which is hereby incorporated by reference in its entirety. Male rats weighing 225-250 g are anesthetized with 4% isoflurane and O₂ and the head of each rat is mounted in a stereotactic frame. The head is held in a horizontal plane. A midline incision is used for exposure, and a 7-8 mm craniectomy is performed on the right cranial vault. The center of the craniectomy is placed at the midpoint between bregma and lambda, ˜3 mm lateral to the midline, overlying the tempoparietal cortex. Animals receive a single impact of 3.1 mm depth of deformation with an impact velocity of 5.8 m/s and a dwell time of 150 ms (moderate-severe injury) at an angle of 10° from the vertical plane using a 6 mm diameter impactor tip, making the impact orthogonal to the surface of the cortex. The impact is made to the parietal association cortex. Sham injuries are performed by anesthetizing the animals, making the midline incision, and separating the skin, connective tissue, and aponeurosis from the cranium. The incision is then closed.

Preparation and intravenous injection of PDACs. Prior to injection, PDACs are thawed, washed and suspended in phosphate buffered saline (PBS) vehicle at a concentration of 2×10⁶ cells/mL. Cells are counted and checked for viability via Trypan blue exclusion. Immediately prior to intravenous injection, PDACs are titrated gently 8-10 times to ensure a homogeneous mixture of cells. PDACs are injected at both 2 and 24 h after CCI injury at 2 different dosages (CCI+2×10⁶ PDACs/kg, and CCI+10×10⁶ PDACs/kg). Therefore, each treatment animal receives 2 separate doses of their assigned PDACs concentration. CCI injury control animals receive PBS vehicle injection alone at the same designated time points as the cell treated animals.

Rat splenectomy. For all experiments completed with rats after splenectomy, male Sprague Dawley rats are anesthetized as described above and placed in the supine position. A small 3 cm incision is made in the left upper quadrant of the abdomen followed by retraction of the spleen and ligation of the splenic hilum. After removal of the spleen the incision is closed with a running suture. The animals are allowed to recover and acclimate for 72 h after splenectomy. All experiments are then completed 72 h after the original splenectomy.

Evan's blue blood brain barrier (BBB) permeability analysis. Seventy two hours after CCI injury, the rats are anesthetized as described above, and 1 mL (4 cm³/kg) of 3% Evan's blue dye in PBS is injected via direct cannulation of the right internal jugular vein. The animals are allowed to recover for 60 min to allow for perfusion of the dye. After this time, the animals are sacrificed via right atrial puncture and perfused with 4% paraformaldehyde. Next, the animals are decapitated followed by brain extraction. The cerebellum is dissected away from the rest of the cortical tissue. The brain is divided through the midline and the mass of each hemisphere (ipsilateral to injury and contralateral to injury) is measured for normalization. Subsequently, each hemisphere is allowed to incubate overnight in 5 mL of formamide at 50° C. to allow for dye extraction. After centrifugation, 100 μl of the supernatant from each sample is transferred to a 96 well plate (in triplicate) and absorbance is measured at 620 nm. All values are normalized to hemisphere weight.

Cortical immunohistochemistry. BBB integrity is further examined by immunostaining for the tight junction protein occluding, and visualization with fluorescent microscopy (DAPI blue for nuclei and FITC green for occludin). Seventy two hours after CCI injury, 4 groups (uninjured, CCI injury alone, CCI injury+2×10⁶ PDACs/kg, and CCI injury+10×10⁶ PDACs/kg) of both rats with intact spleens and rats after splenectomy are sacrificed followed quickly by decapitation. The brains are extracted and both hemispheres (ipsilateral and contralateral to injury) are isolated. The tissue samples are then quickly placed into pre-cooled 2-methylbutane for flash freezing. The samples are transferred to dry ice and stored at −80° C. until the tissue is sectioned. The tissue samples are then placed in Optimal Cutting Temperature compound, for example, Sakura Finetek, Torrance, Calif., and 20 μm cryosections are made through the direct injury area. Direct injury to the vascular architecture is evaluated via staining with an antibody for the tight junction protein occludin (for example, 1:150 dilution, Invitrogen, Carlsbad, Calif.) and appropriate fluorescein isothiocyanate (FITC) conjugated secondary antibody (for example, 1:200 dilution, Invitrogen, Carlsbad, Calif.). After all antibody staining, the tissue sections are counterstained with 4′6-diamidino-2-phenylindole (DAPI) (for example, Invitrogen, Carlsbad, Calif.) for nuclear staining and visualized with fluorescent microscopy.

Splenic immunohistochemistry. In order to track PDACs in vivo, for example, to determine if administered PDACs bypass the pulmonary microvasculature and reach the spleen, 4 groups of rats (uninjured, CCI injury alone, CCI injury+2×10⁶ PDACs/kg, and CCI injury+10×10⁶ PDACs/kg) undergo either sham injury or CCI injury. Next, the two treatment groups receive injections of quantum dot (for example, QDOT, Qtracker cell labeling kit 525 and 800, Invitrogen, Inc., Carlsbad, Calif.) labeled (per manufacturer's suggested protocol) PDACs, 2 and 24 h after CCI injury. Six hours after the second QDOT labeled PDACs infusion, the animals are sacrificed and the spleens removed. The spleens are subsequently placed on a fluorescent scanner (for example, Odyssey Imaging System, Licor Inc., Lincoln, Nebr.) to localize QDOT labeled PDACs. After the scan is completed, the tissue samples are then quickly placed into pre-cooled 2-methylbutane for flash freezing. The samples are transferred to dry ice and stored at −80° C. until use. Next, the tissue samples are placed in Optimal Cutting Temperature compound (for example, Sakura Finetek, Torrance, Calif.) and 10 μM cryosections are made through the spleens. The tissue sections are stained with 4′6-diamidino-2-phenylindole (DAPI) (Invitrogen, Carlsbad, Calif.) for nuclear staining and both the QDOT labeled PDACs and splenocytes are visualized with fluorescent microscopy. Furthermore, hematoxylin and eosin staining is performed per manufacturer's suggested protocol to evaluate splenic architecture.

Splenocyte isolation/measurement of splenic mass. Seventy two hours after injury, the animals undergo splenectomy with measurement of splenic mass. The animals are euthanized at this time. Next, the spleens are morselized using a razor blade, washed with basic media (10% FBS and 1% penicillin/streptomycin in RPMI), crushed, and filtered through a 100 μM filter. The effluent sample from the filter is gently titrated 8-10 times and subsequently filtered through a 40 μm filter to remove any remaining connective tissue. The samples are centrifuged at 1000 g for 3 min. Next the supernatant solutions are removed and the samples are suspended in 3 mL of red blood cell lysis buffer (Qiagen Sciences, Valencia, Calif.) and allowed to incubate on ice for 5 min. Subsequently, the samples are washed twice with basic media and centrifuged using the aforementioned settings. The splenocytes are counted and checked for viability via Trypan blue exclusion.

In vivo splenocyte proliferation assay. The percentage of actively proliferating splenocytes (S phase) at the time of sacrifice is measured using, for example, Click-iT™ EdU Flow Cytometry Assay Kit (Invitrogen, Carlsbad, Calif.) according to the manufacturer's suggested protocol. Briefly, splenocytes are harvested at 72 h, and 20 mM of EdU is added to the cells and allowed to incubate for 2 h. Next, the cells are washed and fixed with 4% paraformaldehyde. Cells are permeabilized using Triton-X100 and then the anti-EdU antibody “cocktail” provided by the manufacturer is added. Finally, the cells are washed followed by the addition of Ribonuclease and CellCycle488-Red stain to analyze DNA content.

In vivo splenocyte apoptosis assay. The percentage of apoptotic splenocytes at the time of sacrifice is measured using, for example, an Annexin V stain (BD Biosciences, San Jose, Calif.) according to the manufacturer's suggested protocol. Briefly, after isolation, splenocytes are washed twice with cold PBS. Next, 1×10⁶ cells are incubated with 54 of Annexin V and 7-Amino-Actinomycin (7-AAD) for 15 min. Flow cytometry is then used to measure the percentage of apoptotic cells. Quantitative PCR RNA is isolated from splenocytes using, for example, RNEasy columns (Qiagen, Valencia, Calif.) according to manufacturer's specifications. Rat reference RNA (Stratagene, La Jolla, Calif.) is used as a positive control. Synthesis of cDNA is performed with M-MLV reverse transcriptase and random hexamers (Promega, Madison, Wis.). Control reactions are performed without reverse transcriptase to control for genomic DNA contamination. qPCR is performed using, for example, an ABI 7500 with 9600 emulation.

### In Vitro Methods

Splenocyte culture. Splenocytes cultured at a density of 7.5×10⁵ cells/mL are allowed to expand for 72 h in growth media (10% FBS, 1% RPMI with vitamins, 1% sodium pyruvate, 0.09% 2-mercaptoethanol, and 1% penicillin/streptomycin in RPMI) stimulated with 2 μg concanavalin A.

Splenocyte characterization. The isolated splenocytes are analyzed with flow cytometry to determine the monocyte, neutrophil, and T cell populations. Monocytes and neutrophils are measured using antibodies to CD200 and CD11b/CD18, respectively. The splenocyte T cell populations are labeled using CD3. CD4, and CD8 antibodies. All staining is completed in accordance with manufacturer's suggested protocol.

Proliferation assay in vitro. The percentage of CD4+ splenocytes actively proliferating (S phase) after culture in stimulated growth media is measured using, for example, Click-iT™ EdU Flow Cytometry Assay Kit (Invitrogen, Carlsbad, Calif.) following the manufacturer's suggested protocol. Briefly, splenocytes are cultured for 72 h as previously described in growth media stimulated with 2 μg concanavalin A at a density of 7.5×10⁵ cells/mL. 20 mM of EdU is added and allowed to incubate for 1 h. Next, the cells are washed with 4% bovine serum in DMEM (4% FBS) and CD4-PE is added to gate the T cell population of interest. After 30 min of incubation, the cells are washed and fixed with 4% paraformaldehyde. Cells are permeabilized using Triton-X100 and then the anti-EdU antibody “cocktail” provided by the manufacturer is added. Finally, the cells are washed followed by the addition of Ribonuclease and CellCycle488-Red stain to analyze DNA content.

Splenocyte cytokine production in vitro. After culture in stimulated growth media, production of the anti-inflammatory cytokines IL-4 and IL-10 was quantified by flow cytometry using, for example, a BD Cytometric Bead Array flex set (BD Biosciences, San Jose, Calif.) following manufacturer's suggested protocol.

### 6.6 Example 6

### Use of PDACs for Tissue Remodeling

This example demonstrates how PDACs can be used to modulate fibrosis and thus remodel tissue.

Using ELISA and multiplex assays, PDAC-conditioned medium was compared with medium conditioned normal human dermal fibroblasts (NHDF) to assess the secretion profiles of the two cell types. PDACs were determined to secrete 60% to 65% more follistatin than the amount of follistatin secreted by the NHDF. PDACs also were determined to secrete 75% to 95% more hepatocyte growth factor (HGF) than the amount of HGF secreted by the NHDF. Additionally, PDACs were determined to secrete matrix metalloproteinase (MMP) 1, MMP2, MMP7, and MMP10.

The determination that PDACs secrete high levels of both follistatin and HGF relative to NHDF, and that PDACs also secrete MMP1, MMP2, MMP7, and MMP10, indicates that PDACs may possess the ability to remodel tissue in vivo, e.g., modulate fibrosis, and thus may be useful in the methods described herein.

### 6.7 Example 7

### Methods of Treatment Using PDACs

### 6.7.1 Treatment of SCI Using PDACs

An individual presents with spinal cord injury (SCI) and is experiencing loss of sensory and/or motor function. The individual is administered 2.5×10⁸ to 1×10¹⁰ CD10⁺, CD34⁻, CD105⁺, CD200⁺ placental stem cells (PDACs) in a 0.9% NaCl solution intravenously. The individual is monitored over the subsequent two weeks to one month to assess reduction in one or more of the symptoms. The individual is additionally monitored over the course of the following year, and PDACs in the same dose are administered as needed, e.g., if symptoms return or increase in severity.

### 6.7.2 Treatment of SCI Using PDACs

An individual presents with spinal cord injury (SCI) and is experiencing loss of sensory and/or motor function. The individual is administered 1×10⁶ to 1×10⁷ CD10⁺, CD34⁻, CD105⁺, CD200⁺ placental stem cells (PDACs) in a 0.9% NaCl at the site of spinal cord injury. The individual is monitored over the subsequent two weeks to one month to assess reduction in one or more of the symptoms. The individual is additionally monitored over the course of the following year, and PDACs in the same dose are administered as needed, e.g., if symptoms return or increase in severity.

### 6.7.3 Treatment of TBI Using PDACs

An individual presents with traumatic brain injury (TBI) and is experiencing memory loss, poor attention/concentration, and/or dizziness/loss of balance. The individual is administered 2.5×10⁸ to 1×10¹⁰ CD10⁺, CD34⁻, CD105⁺, CD200⁺ placental stem cells (PDACs) in a 0.9% NaCl solution intravenously. The individual is monitored over the subsequent two weeks to one month to assess reduction in one or more of the symptoms. The individual is additionally monitored over the course of the following year, and PDACs in the same dose are administered as needed, e.g., if symptoms return or increase in severity.

### 6.7.4 Treatment of TBI Using PDACs

An individual presents with traumatic brain injury (TBI) and is experiencing memory loss, poor attention/concentration, and/or dizziness/loss of balance. The individual is administered 1×10⁶ to 1×10⁷ CD10⁺, CD34⁻, CD105⁺, CD200⁺ placental stem cells (PDACs) in a 0.9% NaCl intracranially. The individual is monitored over the subsequent two weeks to one month to assess reduction in one or more of the symptoms. The individual is additionally monitored over the course of the following year, and PDACs in the same dose are administered as needed, e.g., if symptoms return or increase in severity.

### EQUIVALENTS

The compositions and methods disclosed herein are not to be limited in scope by the specific embodiments described herein. Indeed, various modifications of the compositions and methods in addition to those described will become apparent to those skilled in the art from the foregoing description and accompanying figures. Such modifications are intended to fall within the scope of the appended claims.

Various publications, patents and patent applications are cited herein, the disclosures of which are incorporated by reference in their entireties.

1. A method of treating an individual having or at risk of developing a disease, disorder or condition related to a spinal cord injury or traumatic brain injury, comprising administering to the individual a therapeutically effective amount of CD10⁺, CD34⁻, CD105⁺, CD200⁺ placental stem cells, or culture medium conditioned by said placental stem cells, wherein the therapeutically effective amount is an amount sufficient to cause a detectable improvement in one or more symptoms of said disease, disorder or condition.

2. (canceled)

3. The method of claim 1, wherein said placental stem cells do not express HLA-G, or express CD73, or express OCT-4, or express CD73 and do not express HLA-G, or express CD73 and facilitate the formation of one or more embryoid-like bodies in a population of placental cells comprising said stem cell when said population is cultured under conditions that allow for the formation of an embryoid-like body, or express OCT-4 and facilitate the formation of one or more embryoid-like bodies in a population of placental cells comprising said stem cell when said population is cultured under conditions that allow for the formation of an embryoid-like body.

4. The method of claim 1, wherein the individual has or is at risk of developing a disease, disorder or condition related to a spinal cord injury.

5. The method of claim 4, wherein the spinal cord injury (i) is caused by direct trauma, (ii) is caused by compression by bone fragments, hematoma, or disc material, or (iii) is caused by ischemia from damage or impingement on the spinal arteries.

6. (canceled)

7. (canceled)

8. The method of claim 1, wherein said disease, disorder or condition is (i) spinal shock resulting from a spinal cord injury, (ii) neurogenic shock resulting from a spinal cord injury, (iii) autonomic dysreflexia resulting from a spinal cord injury, or (iv) edema resulting from a spinal cord injury.

9. (canceled)

10. (canceled)

11. (canceled)

12. The method of claim 1, wherein said disease, disorder or condition is selected from the group consisting of central cord syndrome, Brown-Séquard syndrome, anterior cord syndrome, conus medullaris syndrome, and cauda equina syndrome.

13. The method of claim 4, wherein the spinal cord injury is at one or more of the cervical vertebrae, thoracic vertebrae, lumbar vertebrae, sacral vertebrae, cervical cord, thoracic cord, lumbrosacral vertebrae, conus, occiput, or one or more nerves of the cauda equina.

14. (canceled)

15. The method of claim 4, wherein said one or symptoms comprises (i) loss or impairment of motor function, sensory function, or motor and sensory function, in the cervical, thoracic, lumbar or sacral segments of the spinal cord, (ii) loss or impairment of motor function, sensory function, or motor and sensory function, in the arms, trunk, legs or pelvic organs, or (iii) numbness in one or more of dermatomes C1, C2, C1, C4, C5, C6, C7, T1, T2, T3, T4, T5, T6, T7, T8, T9, T10, T11, T12, L1, L2, L3, L4 or L5.

16. (canceled)

17. (canceled)

18. The method of claim 4, wherein the therapeutically effective amount of placental stem cells, or culture medium conditioned by placental stem cells is administered to the individual within 14 days of the spinal cord injury.

19. The method of claim 4, comprising administering a second therapeutic agent to said individual.

20. The method of claim 19, wherein said second therapeutic agent is a corticosteroid, a neuroprotective agent, an immunomodulatory or immunosuppressant agent, or an anticoagulant.

21. The method of claim 1, wherein the individual has or is at risk of developing a disease, disorder or condition related to a traumatic brain injury.

22. The method of claim 21, wherein the traumatic brain injury is an injury to the frontal lobe, parietal lobe, occipital lobe, temporal lobe, brain stem, or cerebellum.

23. The method of claim 21, wherein the traumatic brain injury is a mild traumatic brain injury or a moderate to severe traumatic brain injury.

24. (canceled)

25. The method of claim 21, wherein said symptom is one or more of:
headache, difficulty thinking, memory problems, attention deficits, mood swings and frustration, fatigue, visual disturbances, memory loss, poor attention/concentration, sleep disturbances, dizziness/loss of balance, irritability, emotional disturbances, feelings of depression, seizures, nausea, loss of smell, sensitivity to light and sounds, mood changes, getting lost or confused, and slowness in thinking.

26. The method of claim 21, wherein said symptom is one or more of:
difficulties with attention, difficulties with concentration, distractibility, difficulties with memory, slowness of speed of processing, confusion, perseveration, impulsiveness, difficulties with language processing, difficulties with speech and language, not understanding the spoken word (receptive aphasia), difficulty speaking and being understood (expressive aphasia), slurred speech, speaking very fast or very slow, problems reading, problems writing, difficulties with interpretation of touch, temperature, movement, limb position and fine discrimination, difficulty with the integration or patterning of sensory impressions into psychologically meaningful data, partial or total loss of vision, weakness of eye muscles and double vision (diplopia), blurred vision, problems judging distance, involuntary eye movements (nystagmus), intolerance of light (photophobia), a decrease or loss of hearing, ringing in the ears (tinnitus), increased sensitivity to sounds, loss or diminished sense of smell (anosmia), loss or diminished sense of taste, seizures, convulsions associated with epilepsy, physical paralysis/spasticity, chronic pain, loss of control of bowel and/or bladder, sleep disorders, loss of stamina, appetite changes, dysregulation of body temperature, menstrual difficulties, social-emotional difficulties, dependent behaviors, lack of emotional ability, lack of motivation, irritability, aggression, depression, disinhibition, and lack of awareness.

27. The method of claim 21, comprising administering a second therapeutic agent to said individual.

28. The method of claim 27, wherein said second therapeutic agent is an anti-seizure drug, an antidepressant, amantadine, methylphenidate, bromocriptine, carbamamazapine or amitriptyline.

29. The method of claim 1, wherein the therapeutically effective amount of placental stem cells, or culture medium conditioned by placental stem cells is administered to the individual by a route selected from the group consisting of intravenous, intraarterial, intraperitoneal, intraventricular, intraurethral, intrasternal, intracranial, intramuscular, intrasynovial, intraocular, intravitreal, intracerebral, intracerebroventricular, intrathecal, intraosseous infusion, intravesical, transdermal, intracisternal, epidural, or subcutaneous administration.

30. The method of claim 1, wherein the therapeutically effective amount of placental stem cells, or culture medium conditioned by placental stem cells is administered to the individual directly into the site of the injury.
